(19) United States
(12) Patent Application Publication
(10) Pub. No.: US 2025/0340546 A1
(43) Pub. Date: Nov. 6, 2025

(54) HSD17B13 INHIBITORS

(71) Applicant: Boehringer Ingelheim International GmbH, Ingelheim am Rhein (DE)

(72) Inventors: Marina Kristina WILLWACHER, Biberach (DE); Gary ASPNES, Biberach (DE); Christofer Siegfried TAUTERMANN, Biberach (DE); Thomas VESER, Munderkingen (DE); Lars WORTMANN, Biberach (DE)

(21) Appl. No.: 18/872,318

(22) PCT Filed: Jun. 6, 2023
(86) PCT No.: PCT/EP2023/065025

§ 371 Date: Dec. 6, 2024

(30) Foreign Application Priority Data
Jun. 9, 2022 (EP) 22177977.0

(51) Int. Cl. C07D 417/06; A61K 31/427; A61K 31/433; A61K 31/454; A61K 31/4709; A61K 31/513; A61K 31/517; A61K 31/522; A61K 31/5377; C07D 413/06; C07D 473/04; C07D 513/04

(52) U.S. Cl. CPC C07D 417/06; A61K 31/427; A61K 31/433; A61K 31/454; A61K 31/4709; A61K 31/513; A61K 31/517; A61K 31/522; A61K 31/5377; C07D 413/06; C07D 473/04; C07D 513/04

(57) ABSTRACT

The present invention encompasses heteroaryl substituted 2,6-difluorophenol compounds of formula (I), wherein the groups A1 to A3, and Z have the meanings given in the claims and specification, their use in pharmaceutical compositions which contain these compounds and their use as medicaments, especially to interfere with the progression of liver disease from steatosis to later stages of nonalcoholic steatohepatitis, fibrosis, and cirrhosis.

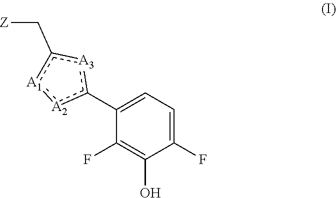

### FIELD OF THE INVENTION

The present invention relates to heteroaryl substituted 2,6-difluorophenol compounds of formula (I),

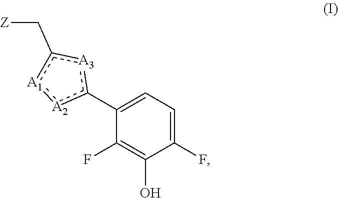

wherein A₁ to A₃, and Z have the meanings given in the claims and specification. Additionally disclosed are their use as inhibitors of HSD17B13, pharmaceutical compositions which contain said compounds and their use as medicaments, especially as agents for interfering with steatosis.

### BACKGROUND OF THE INVENTION

WO 2021/211974 and WO 2022/020714 disclose thiophene-carboxamide HSD17B13 inhibitors.

WO 2022/020730 discloses quinazolinone HSD17B13 inhibitors.

HSD17B13 is a member of the 17b-hydroxysteroid dehydrogenases family of oxidoreductase enzymes that collectively act on a range of lipid substrates. In humans, HSD17B13 mRNA is most highly expressed in the liver, primarily in hepatocytes. Within the cell, HSD17B13 is associated with lipid droplets (Su et al, Proc National Acad Sci. 111:11437-11442, 2014).

The physiological function of HSD17B13 is uncertain, and multiple substrates, including estradiol, retinol, and leukotriene B4, have been identified using an in vitro enzyme assay system in which NAD⁺ (nicotinamide adenine dinucleotide, oxidized form) acted as co-substrate (Abdul-Husn et al, The New England Journal of Medicine. 378:1096-1106, 2018).

Loss of function (LoF) genetic variants in humans provide evidence for a role of HSD17B13 activity in mediating risk of certain liver diseases. The presence of the single nucleotide polymorphism (SNP) rs72613567 encoding a truncated, enzymatically inactive protein, has been associated with liver diseases such as liver fibrosis or non-alcoholic steatohepatitis (NASH).

The SNP rs72613567 SNP also mitigates the increased risk of liver disease.

The SNP rs72613567 was found to occur at a lower frequency in liver transplant recipients than in healthy controls.

The SNP rs62305723 encoding a HSD17B13 LoF variant has been associated with decreased severity of NASH (Ma et al, Hepatology 69:1504-1519, 2018).

The SNP rs80182459 encoding a probable LoF variant has been found to be less frequent in certain patients with chronic liver disease (Kozlitina et al, The New England Journal of Medicine 379:1876-1877, 2018).

SNP rs6834314 which is in high linkage with SNP rs72613567 was also found to be associated with fatty liver disease.

A hepatocyte-directed small interfering RNA (siRNA) designed to deplete HSD17B13 in human liver was found in 5 patients with fatty liver to decrease serum alanine aminotransferase (ALT) activity, a biomarker of liver damage.

In view of the data mentioned above it is desirable to provide potent HSD17B13 inhibitors.

According to the present invention, “HSD17B13 inhibitor(s)” means compounds which inhibit HSD17B13 in the test shown in examples 4 and 6.

### DETAILED DESCRIPTION OF THE INVENTION

Compounds of formula (I), wherein the groups A1 to A3 and Z have the meanings given hereinafter, were not known to act as HSD17B (17β-Hydroxysteroid dehydrogenase) inhibitors selective for HSD17B13 as shown in example 12 by the comparative biochemical human IC₅₀ data for HSD17B11. Thus, the compounds according to the invention may be used for example for the treatment of steatosis such as non-alcoholic fatty liver disease (NAFLD) or non-alcoholic steatohepatitis (NASH).

The present invention therefore relates to a compound of formula (I), or a salt thereof,

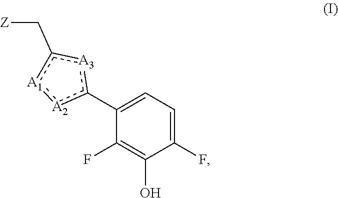

a) wherein Z-* is

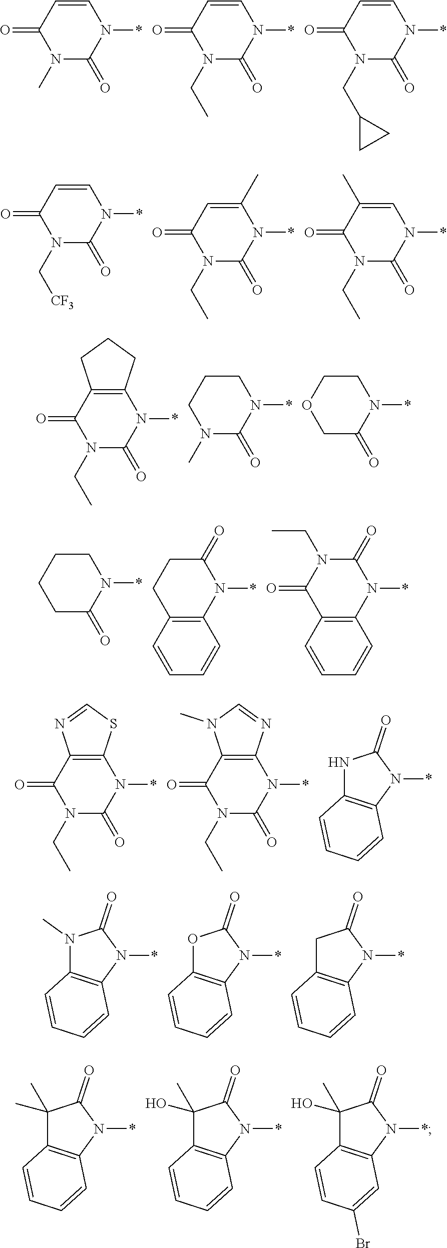

and b) wherein the structure

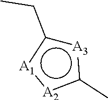

is selected from the group of structures consisting of

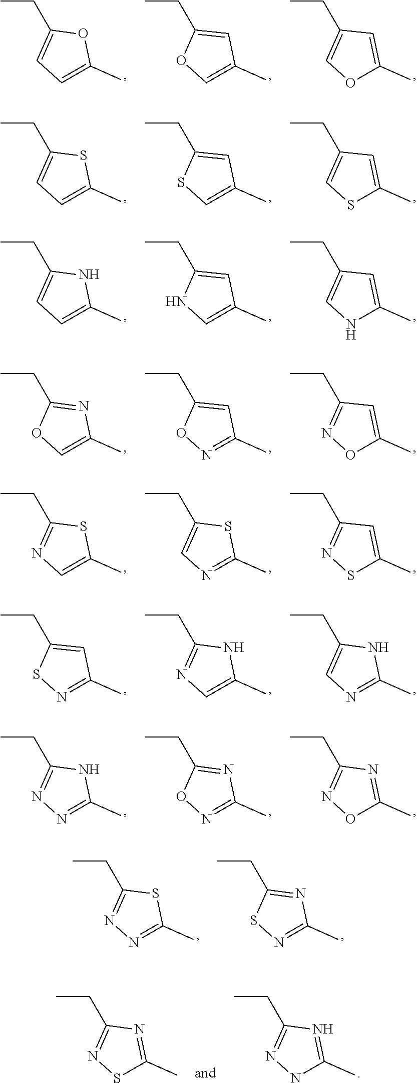

A skilled artisan is aware that several of heteroaryl groups for example can be described in form of different tautomers, i.e. pyrazoles, triazoles, imidazoles. Thus, the compounds of the present invention may exist as tautomeres. For example, any compound of the present invention which contains a pyrazole moiety as a heteroaryl group can exist as a 1H tautomer, or a 2H tautomer, or even a mixture in any amount of the two tautomers, or a triazole moiety can exist as a 1H tautomer, a 2H tautomer or a 4H tautomer, or even a mixture in any amount of said 1H, 2H or 4H tautomers, namely:

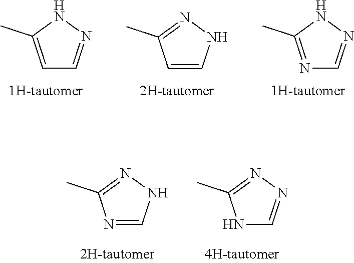

The present invention includes all possible tautomers of the compounds of the present invention as single tautomers, or as any mixture of said tautomers, in any ratio.

In particular, the structure

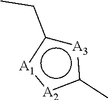

can be

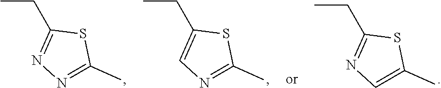

In one group of compounds according to the invention the structure of

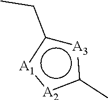

is selected from the group of structures consisting of

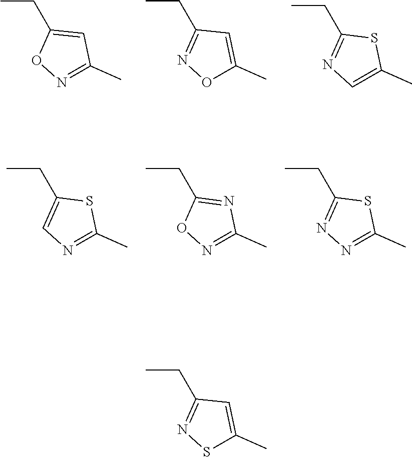

In another group of compounds of the invention the structure of

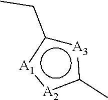

is selected from the group of structures consisting of

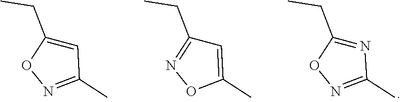

In yet another group of compounds of the invention the structure

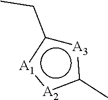

is selected from the group of structures consisting of

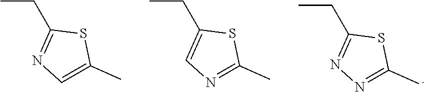

Z is preferably

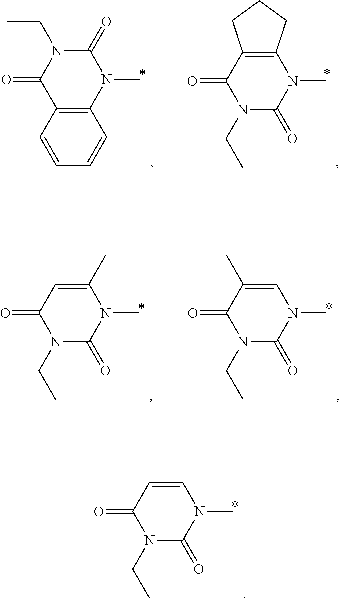

The most preferred structures of Z are

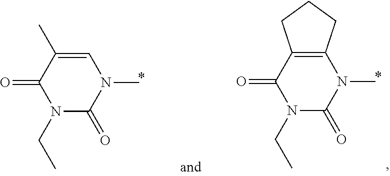

The present invention is directed to compounds of formula (I) or salts thereof which interfer with lipogenesis wherein the selective inhibition of HSD17B13 is of therapeutic benefit, including but not limited to the treatment of non-alcoholic steatohepatitis. Thus, in another aspect the invention a compound of formula (I) or a pharmaceutically acceptable salt thereof is used as a medicament. The invention also relates to a compound of formula (I) or a pharmaceutically acceptable salt thereof for use in a method of treatment of the human or animal body.

Of particular interest are the use of one of the compounds of formula (I) or a salt thereof in the treatment of metabolic disorders. Therefore, another aspect of the invention is the use of a compound of formula (I) or a pharmaceutically acceptable salt thereof for preparing a pharmaceutical composition comprising at least one compound of formula (I) or a pharmaceutically acceptable salt thereof and a pharmaceutically acceptable carrier for the treatment of one of said disorders. Particularly preferred is their use in preparing a pharmaceutical composition to modulate metabolic disorders in the human or animal body.

Considered in the context of the present invention is also a method of treating a liver disease, metabolic disease, or cardiovascular disease using a compound disclosed herein, or a pharmaceutically acceptable salt, solvate or stereoisomer thereof, in combination with an additional therapeutic agent.

In some embodiments, the additional therapeutic agent is used for the treatment of diabetes or diabetes related disorder or conditions.

In some instances, the additional therapeutic agent comprises a statin, an insulin sensitizing drug, an insulin secretagogue, an alpha-glucosidase inhibitor, a GLP agonist, a THR beta agonist, a PDE inhibitor, a DPP-4 inhibitor (such as sitagliptin, vildagliptin, saxagliptin, linagliptin, anagliptin, teneligliptin, alogliptin, gemigliptin or dutogliptin) a catecholamine (such as epinephrine, norepinephrine or dopamine), a peroxisome-proliferator-activated receptor (PPAR)-gamma agonist (e.g. thiazolidinedione (TZD) [such as pioglitazone, rosiglitazone, rivoglitazone, or troglitazone], aleglitazar, farglitazar, muraglitazar or tesaglitazar, peroxisome-proliferator-activated receptor (PPAR)-alpha agonist, a peroxisome-proliferator-activated receptor (PPAR)-delta agonist, a farnesoid X receptor (FXR) agonist (e.g. obeticholic acid), or a combination thereof.

In some cases, the statin is an HMG-COA reductase inhibitor.

In other instances, additional therapeutic agents include fish oil, fibrate, vitamins such as niacin, retinoic acid (e.g. 9-cis retinoic acid), nicotinamide ribonucleoside or its analogs thereof, or combinations thereof. In other instances, additional therapeutic agents include ACC inhibitors, FGF19 and FGF21 mimics, CCR3/CCR5 antagonists, or combinations thereof.

In some embodiments, the additional therapeutic agent is vivitrol.

In some embodiments, the additional therapeutic agent is a statin, such as an HMG-COA reductase inhibitor, fish oil, fibrate, niacin, or a combination thereof. In other instances, the additional therapeutic agent is a dyslipidemia drug that prevents lipid absorption such as orlistat.

In some embodiments, the additional therapeutic agent is a vitamin such as retinoic acid or tocopheryl acetate for the treatment of diabetes and diabes related disorder or condition such as lowering elevated body weight and/or lowering elevated blood glucose from food intake. In some embodiments, the additional therapeutic agent is a glucose-lowering agent. In some embodiments, the additional therapeutic agent is an anti-obesity agent.

In some embodiments, the additional therapeutic agent is selectected from among a peroxisome proliferator activated receptor (PPAR) agonist (gamma, dual or pan), a dipeptidyl peptidase (IV) inhibitor, a glucagon-like peptide-1 (GLP-1) analog, insulin or an insulin analog, an insulin secretagogue, a sodium glucose co-transporter 2 (SGLT2) inhibitor, a Glucophage, a human amylin analog, a biguanide, an alpha-glucosidase inhibitor, a meglitinide, a thiazolidinedione and sulfonylurea.

In some embodiments, the additional therapeutic agent is a lipid-lowering agent.

In some embodiments, the additional therapeutic agent is an antioxidant, corticosteroid such as budesonide, anti-tumor necrosis factor (TNF), or a combination thereof.

In some embodiments the additional therapeutic agent is administered at the same time as the compound disclosed herein.

In some embodiments, the additional therapeutic agent is administered less frequently than the compound disclosed herein.

In some embodiments, the additional therapeutic agent is administered more frequently than the compound disclosed herein.

In some embodiments, the additional therapeutic agent is administered prior than the administration of the compound disclosed herein.

In some embodiments, the additional therapeutic agent is administered after the administration of the compound disclosed herein.

In a treatment of a mammal, a compound according to the invention can be administered before, after or together with at least one other active substance or agent such as a diuretic, antihypertensive, lipid-lowering or antidiabetic agent.

### Formulations

Suitable preparations for administering the compounds of the invention will be apparent to those with ordinary skill in the art and include for example tablets, pills, capsules, suppositories, lozenges, troches, solutions, elixirs, syrups, sachets, emulsions, inhalatives or dispersible powders. Preferred solutions are solutions for injection (s.c., i.v., i.m.) or solutions for infusion (injectables).

The content of the pharmaceutically active compound needs to be in amounts which are sufficient to achieve the dosage range specified below, for example in the range from 0.10 to 90 wt.-%, preferably 0.5 to 50 wt.-% of the composition as a whole. The doses specified may, if necessary, be given several times a day.

Suitable tablets may be obtained, for example, by mixing the active substance(s) of the invention with known excipients, for example inert diluents, carriers, disintegrants, adjuvants, surfactants, binders and/or lubricants.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities, the core may consist of multiple layers. Similarly, the tablet coating may consist of multiple layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids and transferred into injection vials or ampoules or infusion bottles.

Capsules may for example be prepared by mixing the active substance with an inert carrier such as lactose or sorbitol and packing them into gelatine capsules.

Suitable suppositories may be made for example by mixing with carriers provided for this purpose such as neutral fats or polyethyleneglycol or derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by an oral or transdermal route, most preferably by oral route. For oral administration the tablets may of course contain, apart from the above-mentioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In aqueous suspensions the active substances may be combined with various flavour enhancers or colourings in addition to the excipients mentioned above.

For parenteral use, a solution of an active substance with suitable liquid carriers may be used.

The total amount of the active ingredient of formula (I) to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

It may sometimes be necessary to depart from the amounts specified, depending on a person's body weight, age, the route of administration, severity of the disease, individual response to a drug, nature of the formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering a higher dosis, it may be advisable to divide the higher dosis into a number of smaller doses spread over the day.

“Pharmaceutically acceptable salts” as used herein refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

### Preparation of the Compounds According to the Invention

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

The compounds according to the invention are named in accordance with CAS rules using the software MarvinSketch (Chemaxon).

The compounds according to the invention are prepared by the methods of synthesis described hereinafter in which the substituents of the general formulae have the meanings given herein before. These methods are intended as an illustration of the invention without restricting its subject matter and the scope of the compounds claimed to these examples. Where the preparation of starting compounds is not described, they are commercially obtainable or may be prepared analogously to known compounds or methods described herein. Substances described in the literature are prepared according to the published methods of synthesis.

### Overview

### Synthesis of Compounds:

The compounds of the present invention can be prepared as described in the following section. The schemes and the procedures described below illustrate general synthetic routes to the compounds of general formula (I) of the invention and are not intended to be limiting. It is clear to the person skilled in the art that the order of transformations as exemplified in the schemes can be modified in various ways. The order of transformations exemplified in the schemes is therefore not intended to be limiting. In addition, interconversion of any of the substituents can be achieved before and/or after the exemplified transformation. These modifications can be achieved by introduction of protecting groups, cleavage of protecting groups, exchange, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example P.G.M. Wuts and T.W. Greene in “Protective Groups in Organic Synthesis”, 4th edition, Wiley 2006). Specific examples are described in the subsequent paragraphs. Further, it is possible that two or more successive steps may be performed without work-up between said steps, e.g. in a “one-pot” reaction, as it is well-known to the person skilled in the art.

The synthesis of heteroaryl substituted 2,6-difluorophenol compounds according to the present invention are preferably carried out according to the general synthetic sequence, shown in schemes 1-3.

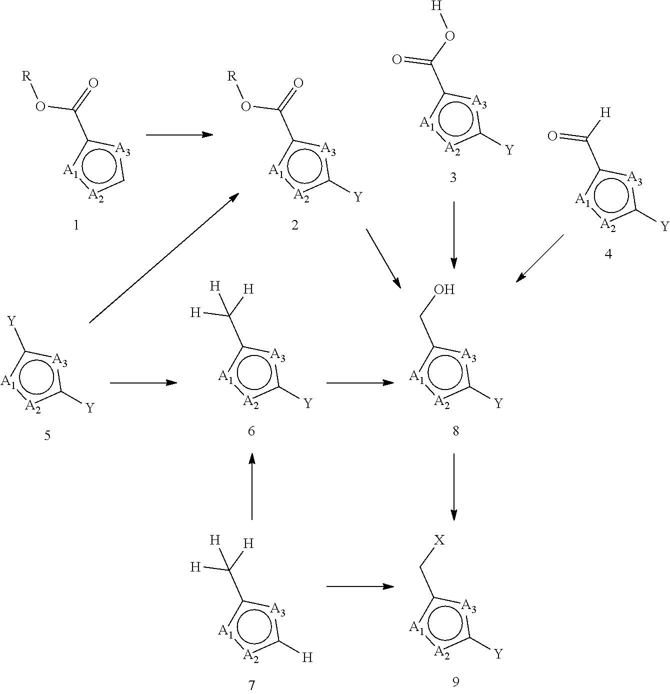

Scheme 1: Route for the preparation of compounds of the general formula 8 and 9, wherein A1, A2 and A3 have the same meaning as given for the formula (I), supra, X has the meaning of Cl, Br, I, mesylate or triflate and Y has the meaning of Cl, Br or I and R has the meaning of alkyl.

### Preparation of Starting Materials (Scheme 1):

The heterocyclic compounds of the general formulas 1, 2, 3, 4, 5, 6 or 7 (Scheme 1) are commercially available or described in the literature.

### Step 1→2 (Scheme 1)

### Halogenation Reaction

The conversion of compounds of the general formula 1 to compounds of the formula 2 is known to the skilled person. For Y═Br the reaction can be performed with reagents such as bromine, N-Bromosuccinimide or copper (11) bromide. For Y═Cl the reaction can be performed with reagents such as N-chloro-succinimide or chlorine. For Y═I the reaction can be performed with reagents such as iodine or N-iodo-succinimide.

### Step 2→8 (Scheme 1)

### Reduction of Ester to Alcohol

The conversion of compounds of the general formula 2 to alcohols of the formula 8 is known to the skilled person and can be performed with reagents such as lithium aluminium tetrahydride, sodium tetrahydridoborate, calcium borohydride, diisobutylaluminium hydride.

### Step 3→8 (Scheme 1)

### Reduction of Carboxylic Acid to Alcohol

The conversion of compounds of the general formula 3 to alcohols of the formula 8 is known to the skilled person and can be performed with reagents such as lithium aluminium tetrahydride.

### Step 4→8 (Scheme 1)

### Reduction of Aldehyde to Alcohol

The conversion of compounds of the general formula 4 to alcohols of the formula 8 is known to the skilled person and can be performed with reagents such as sodium tetrahydroborate or lithium aluminium hydride.

### Step 5→6 (Scheme 1)

### Metal-Catalized Methylation Reaction

The conversion of compounds of the general formula 5 to compounds of the general formula 6 is known to the skilled person and can be performed for example under Suzuki conditions with reagents such as trimethylboroxin, a palladium catalyst such as (1,1′-bis(diphenylphosphino) ferrocene) palladium (II) dichloride or tetrakis (triphenylphosphine) palladium (0) and a base such as potassium carbonate in an organic solvent such as DMF or dioxane.

### Step 7→6 (Scheme 1)

### Halogenation Reaction

The conversion of compounds of the general formula 7 to compounds of the general formula 6 is known to the skilled person. For Y═Br the reaction can be performed with reagents such as bromine, N-Bromosuccinimide or copper (11) bromide. For Y═Cl the reaction can be Fperformed with reagents such as N-chloro-succinimide or chlorine. For Y═I the reaction can be performed with reagents such as iodine or N-iodo-succinimide.

### Step 6→8 (Scheme 1)

### Hydroxylation of Methyl Group

The conversion of compounds of the general formula 6 to alcohols of the formula 8 is known to the skilled person and can be performed with reagents such as selenium (IV)dioxide or tert-butylhydroperoxide/manganese triacetate.

### Step 8→9 (Scheme 1)

### Conversion of Hydroxy to Halogen (Br, Cl, I), Mesylate or Triflate

The transformation of alcohols 8 to a halogenated compound of formula 9 can be performed (for X═Cl) for example using chlorinating reagents such as thionyl chloride, mesylchloride/triethylamine or triphenylphosphine/tetrachloromethane. For X═Br reagents such as phosphorus tribromide, trimethylsilyl bromide, hydrogen bromide, carbon tetrabromide/triphenylphosphine or boron tribromide/triphenylphosphine can be used. For X═I reagents such as boron trifluoride diethyl etherate/potassium iodide, 1H-imidazole/iodine/triphenylphosphine can be used. For X═mesylate reagents such as mesyl chloride and a base such as triethylamine can be used. For X═triflate reagents such as triflic anhydride and a base such as pyridine can be used.

### Step 7→9 (Scheme 1)

### Radical Halogenation of Methyl Group

The conversion of compounds of the general formula 7 to halides of the formula 9 is known to the skilled person. Typically, a radical starter such as dibenzoyl peroxide or 2,2′-azobis(isobutyronitrile) and a halogenation reagent are used. For X═Br the reaction can be performed with halogentation reagents such as N-Bromosuccinimide. For X═Cl the reaction can be performed with halogenation reagents such as N-chloro-succinimide.

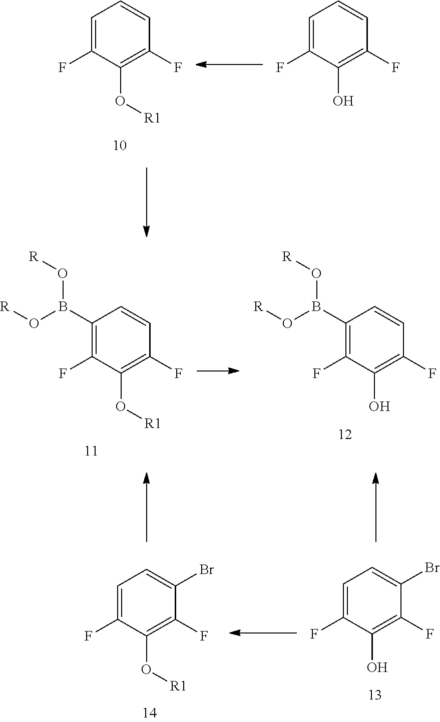

Scheme 2: Route for the preparation of compounds of the general formulas 11, 12 and 14, wherein R has the meaning of hydrogen or alkyl and R1 can be a protecting group known to the person skilled in the art (see for example T. W. Greene and P. G. M. Wuts in Protective Groups in Organic Synthesis, 3^{rd} edition, Wiley 1999).

### Preparation of Starting Materials (Scheme 2):

Compounds of the general formulas 10, 11, 12, 13 or 14 (Scheme 2) are commercially available or described in the literature. 2,6-Difluorophenol is commercially available.

### Step 2,6-Difluorophenol→10 (Scheme 2)

### Introduction of Phenol Protecting Group

The phenol group of 2,6-difluorophenol can be masked with a suitable protecting group R1 leading to compounds of the general formula 10. The reaction conditions for introduction of such suitable protecting groups R1 are known to the skilled person (see for example Green, Wuts, “Protective groups in organic synthesis” 1999, John Wiley & Sons and references therein). Preferably benzyl, para-methoxybenzyl and 3,4-methoxybenzyl are used as protective groups during the synthesis.

### Step 10→11 (Scheme 2)

### Introduction of Boronic Acid or Boronic Acid Ester

The conversion of compounds of the general formula 10 to boronic acid derivatives of the formula 11 is known to the skilled person. For R═H the reaction can be performed with reagents such as triisopropyl borate and a base such as n-butyllithium followed by an acidic workup of the reaction. For R—R═—C(CH₃)₂—C(CH₃)₂— the reaction can be performed with reagents such as bis(pinacol) diborane and a catalyst such as Pt (N,N′-dicyclohexylimidazol-2-ylidene) (divinyltetramethylsiloxane).

### Step 11→12 (Scheme 2)

### Removal of Phenol Protecting Group

Removal of the protecting group R1 from compounds of formula 11 leads to compounds of formula 12. The reaction conditions for removal of such suitable protecting groups R1 are known to the skilled person (see for example Green, Wuts, “Protective groups in organic synthesis” 1999, John Wiley & Sons and references therein). Preferably benzyl, para-methoxybenzyl and 3,4-methoxybenzyl are used as protective groups during the synthesis which can be removed by hydrogenation.

### Step 13→14 (Scheme 2)

### Introduction of Phenol Protecting Group

Compounds of the general formula 13 can be masked with a suitable protecting group R1 leading to compounds of the general formula 14. The reaction conditions for introduction of such suitable protecting groups R1 are known to the skilled person (see for example Green, Wuts, “Protective groups in organic synthesis” 1999, John Wiley & Sons and references therein). Preferably benzyl, para-methoxybenzyl and 3,4-methoxybenzyl are used as protective groups during the synthesis.

### Step 14→11 (Scheme 2)

### Introduction of Boronic Acid Ester

The conversion of compounds of the general formula 14 to boronic acid derivatives of the formula 11 is known to the skilled person. For R—R═—C(CH₃)₂—C(CH₃)₂— the reaction can be performed with reagents such as bis(pinacol) diborane, a catalyst such as palladium (II) [1,1′-bis(diphenylphosphanyl) ferrocene]dichloride and a base such as potassium acetate.

### Step 13→12 (Scheme 2)

### Introduction of Boronic Acid Ester

The conversion of compounds of the general formula 13 to boronic acid derivatives of the formula 12 is known to the skilled person. For R—R═—C(CH₃)₂—C(CH₃)₂— the reaction can be performed with reagents such as bis(pinacol) diborane, a catalyst such as palladium (II) [1,1′-bis(diphenylphosphanyl) ferrocene]dichloride and a base such as potassium acetate.

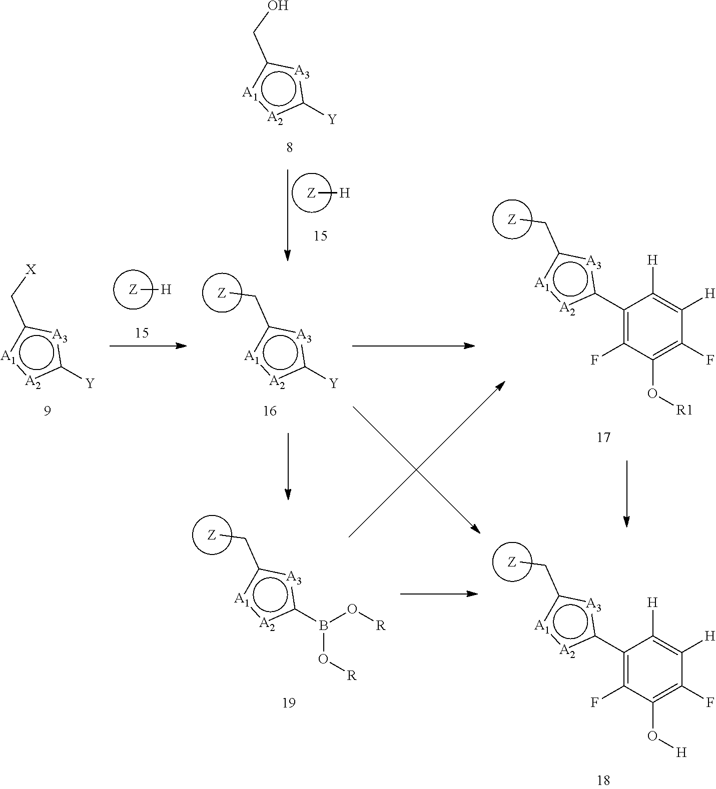

Scheme 3: Route for the preparation of compounds of the general formula 18, wherein A1, A2, A3 and Z have the same meaning as given for the formula (I), supra, X has the meaning of hydroxy, Cl, Br, I, mesylate or triflate, Y has the meaning of Cl, Br or I, R has the meaning of hydrogen or alkyl. In addition, R1 can be a a protecting group known to the person skilled in the art (see for example T. W. Greene and P. G. M. Wuts in Protective Groups in Organic Synthesis, 3^{rd} edition, Wiley 1999).

### Preparation of Starting Materials (Scheme 3):

Amide compounds of the general formulas 15 (Scheme 3) are commercially available, are described in the literature or can be prepared in analogy to literature procedures.

### Step 8+15→16 (Scheme 3)

### Mitsunobu Reaction

The conversion of compounds of the general formula 8 and compounds of the general formula 15 to derivatives of the formula 16 is known to the skilled person. The reaction can be performed under Mitsunobu conditions using reagents such as di-isopropyl azodicarboxylate/triphenylphosphine, di-tert-butyl azodicarboxylate/triphenylphosphine or dietylazodicarboxylate/triphenylphosphine.

### Step 9+15→16 (Scheme 3)

### Alkylation of Amide

The conversion of compounds of the general formula 9 and compounds of the general formula 15 to derivatives of the formula 16 is known to the skilled person. For X═Cl, Br or I the reaction can be performed with a base such as potassium carbonate, caesium carbonate, sodium hydride or LDA in solvents such as DMF, DMSO or acetonitrile.

### Step 16+11→17 (Scheme 3)

Palladium Catalyzed Reaction with Boronic Acids

Heteroaryl halides of formula 16 can be reacted with a boronic acid derivative 11 to give a compound of formula 17. The boronic acid derivative may be a boronic acid (R═H) or an ester of the boronic acid, e.g. its iropropyl ester (R═—CH(CH₃)₂), preferably an ester derived from pinacol in which the boronic acid intermediate forms a 2-aryl-4,4,5,5-tretramethyl-1,3,2,-dioxoborolane (R—R═—C(CH₃)₂—C(CH₃)₂—). The coupling reaction is catalyzed by palladium catalysts, e.g. by a Pd (0) catalyst like tetrakis (triphenylphosphine) palladium (0) [Pd(PPh₃)₄], tris(dibenzylidideneacetone) di-palladium (0) [Pd₂(dba)₃], or by Pd (II) catalysts like dichlorobis(triphenylphosphine)-palladium (II) [Pd(PPh₃)₂Cl₂], XPhos Pd G2, Pd-Peppsi 2Me-Ipent Cl, palladium (II) acetate and triphenylphosphine or by [1,1′-bis(diphenylphosphino) ferrocene]palladium dichloride.

The reaction is preferably carried out in a mixture of a solvent like 1,2-dimethoxymethane, dioxane, DMF, DME, THF, ethanol or isopropanol with water and in the presence of a base like potassium carbonate, sodium bicarbonate, potassium acetate or potassium phosphate. (review: D.G. Hall, Boronic Acids, 2005 WILEY-VCH Verlag GmbH & Co. KGaA, Weinheim, ISBN 3-527-30991-8 and references therein).

The reaction is performed at temperatures ranging from room temperature (i.e. approx. 20° C.) to the boiling point of the respective solvent. Further on, the reaction can be performed at temperatures above the boiling point using pressure tubes and a microwave oven. The reaction is preferably completed after 1 to 36 hours of reaction time.

### Step 17→18 (Scheme 3)

### Removal of Phenol Protecting Group

Removal of the protecting group R1 from compounds of formula 17 leads to compounds of formula 18. The reaction conditions for removal of such suitable protecting groups R1 are known to the skilled person (see for example Green, Wuts, “Protective groups in organic synthesis” 1999, John Wiley & Sons and references therein). Preferably benzyl, para-methoxybenzyl and 3,4-methoxybenzyl are used as protective groups during the synthesis which can be removed by hydrogenation.

### Step 16→19 (Scheme 3)

### Introduction of Boronic Acid Ester

The conversion of compounds of the general formula 16 to boronic acid derivatives of the formula 19 is known to the skilled person. For R—R═—C(CH₃)₂—C(CH₃)₂— the reaction can be performed with reagents such as bis(pinacol) diborane, a catalyst such as palladium (II) [1,1′-bis(diphenylphosphanyl) ferrocene]dichloride and a base such as potassium acetate.

### Step 19+13→18 (Scheme 3)

Palladium Catalyzed Reaction with Boronic Acids

Aryl halides of formula 13 can be reacted with a boronic acid derivative 19 to give a compound of formula 18. The boronic acid derivative may be a boronic acid (R═H) or an ester of the boronic acid, e.g. its iropropyl ester (R═—CH(CH₃)₂), preferably an ester derived from pinacol in which the boronic acid intermediate forms a 2-aryl-4,4,5,5-tretramethyl-1,3,2,-dioxoborolane (R—R═—C(CH₃)₂—C(CH₃)₂—).

The coupling reaction is catalyzed by palladium catalysts, e.g. by Pd (0) catalyst like tetrakis (triphenylphosphine) palladium (0) [Pd(PPh₃)₄], tris(dibenzylidideneacetone) di-palladium (0) [Pd₂ (dba)₃], or by Pd (II) catalysts like dichlorobis(triphenylphosphine)-palladium (II) [Pd(PPh₃)₂Cl₂], XPhos Pd G2, Pd-Peppsi 2Me-Ipent Cl, palladium (II) acetate and triphenylphosphine or by [1,1′-bis(diphenylphosphino) ferrocene]palladium dichloride.

The reaction is preferably carried out in a mixture of a solvent like 1,2-dimethoxymethane, dioxane, DMF, DME, THF, ethanol or isopropanol with water and in the presence of a base like potassium carbonate, sodium bicarbonate, potassium acetate or potassium phosphate (review: D.G. Hall, Boronic Acids, 2005 WILEY-VCH Verlag GmbH & Co. KGaA, Weinheim, ISBN 3-527-30991-8 and references therein).

The reaction is performed at temperatures ranging from room temperature (i.e. approx. 20° C.) to the boiling point of the respective solvent. Further on, the reaction can be performed at temperatures above the boiling point using pressure tubes and a microwave oven. The reaction is preferably completed after 1 to 36 hours of reaction time.

### Step 16+12→18 (Scheme 3)

Palladium Catalyzed Reaction with Boronic Acids

Heteroaryl halides of formula 16 can be reacted with a boronic acid derivative 12 to give a compound of formula 18. The boronic acid derivative may be a boronic acid (R═H) or an ester of the boronic acid, e.g. its iropropyl ester (R═—CH(CH₃)₂), preferably an ester derived from pinacol in which the boronic acid intermediate forms a 2-aryl-4,4,5,5-tretramethyl-1,3,2,-dioxoborolane (R—R═—C(CH₃)₂—C(CH₃)₂—).

The coupling reaction is catalyzed by palladium catalysts, e.g. by Pd (0) catalyst like tetrakis (triphenylphosphine) palladium (0) [Pd(PPh₃)₄], tris(dibenzylidideneacetone) di-palladium (0) [Pd₂ (dba)₃], or by Pd (II) catalysts like dichlorobis(triphenylphosphine)-palladium (II) [Pd(PPh₃)₂Cl₂], XPhos Pd G2, Pd-Peppsi 2Me-Ipent Cl, palladium (II) acetate and triphenylphosphine or by [1,1′-bis(diphenylphosphino) ferrocene]palladium dichloride.

The reaction is preferably carried out in a mixture of a solvent like 1,2-dimethoxymethane, dioxane, DMF, DME, THF, ethanol or isopropanol with water and in the presence of a base like potassium carbonate, sodium bicarbonate, potassium acetate or potassium phosphate (as reviewed in D.G. Hall, Boronic Acids, 2005 WILEY-VCH Verlag GmbH & Co. KGaA, Weinheim, ISBN 3-527-30991-8 and references therein).

The reaction is performed at temperatures ranging from room temperature (i.e. approx. 20° C.) to the boiling point of the respective solvent. Further on, the reaction can be performed at temperatures above the boiling point using pressure tubes and a microwave oven. The reaction is preferably completed after 1 to 36 hours of reaction time.

### Step 19+14→17 (Scheme 3)

Palladium Catalyzed Reaction with Boronic Acids

Aryl halides of formula 14 can be reacted with a boronic acid derivative 19 to give a compound of formula 17. The boronic acid derivative may be a boronic acid (R═H) or an ester of the boronic acid, e.g. its iropropyl ester (R═—CH(CH₃)₂), preferably an ester derived from pinacol in which the boronic acid intermediate forms a 2-aryl-4,4,5,5-tretramethyl-1,3,2-dioxoborolane (R—R═—C(CH₃)₂—C(CH₃)₂—).

The coupling reaction is catalyzed by palladium catalysts, e.g. by Pd (0) catalyst like tetrakis (triphenylphosphine) palladium (0) [Pd(PPh₃)₄], tris(dibenzylidideneacetone) di-palladium (0) [Pd₂ (dba)₃], or by Pd (II) catalysts like dichlorobis(triphenylphosphine)-palladium (II) [Pd(PPh₃)₂Cl₂], XPhos Pd G2, Pd-Peppsi 2Me-Ipent Cl, palladium (II) acetate and triphenylphosphine or by [1,1′-bis(diphenylphosphino) ferrocene]palladium dichloride.

The reaction is preferably carried out in a mixture of a solvent like 1,2-dimethoxymethane, dioxane, DMF, DME, THF, ethanol or isopropanol with water and in the presence of a base like potassium carbonate, sodium bicarbonate, potassium acetate or potassium phosphate (review: D.G. Hall, Boronic Acids, 2005 WILEY-VCH Verlag GmbH & Co. KGaA, Weinheim, ISBN 3-527-30991-8 and references therein).

The reaction is performed at temperatures ranging from room temperature (i.e. approx. 20° C.) to the boiling point of the respective solvent. Further on, the reaction can be performed at temperatures above the boiling point using pressure tubes and a microwave oven. The reaction is preferably completed after 1 to 36 hours of reaction time.

### General Preparation Method for Compounds of Formula (I):

The present invention discloses the following method or process to prepare compounds of general formula (I) using compounds X1 and X2:

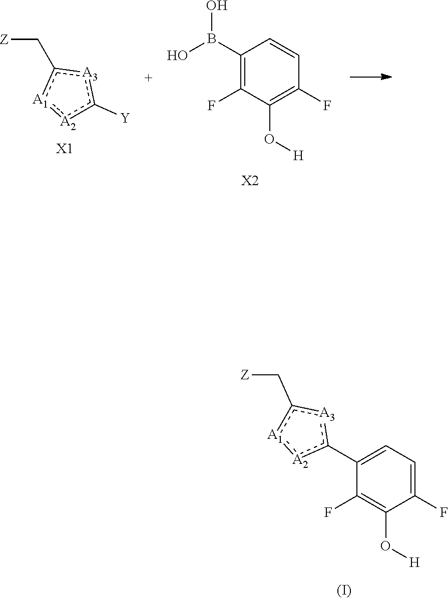

Said method to obtain compounds of general formula (I) is characterized in that
In the compounds of general formula X1, Z, A1, A2 and A3 have the same meaning as defined for the compounds of the general formula (I),.
the compounds of general formula X1, wherein Y can be Br, I or Cl, are reacted with a compound of general formula X2 using a palladium catalyst and a base; and
the reaction takes place in a solvent or solvent mixture at a temperature between ambient temperature and the boiling point of the solvent, preferably between 50° C. and 120° C.
The preparation of the compounds of the general formula (I) can be performed in an aprotic or protic solvent or a solvent mixture, preferably in 1,4-dioxane, tetrahydrofurane, or ethanol/water.
Preferred bases which can be used for the preparation of compounds of the general formula (I) are sodium carbonate or cesium carbonate.
Preferred palladium catalysts for preparation of compounds of general formula (I) are
   chloro(2-dicyclohexylphosphino-2′,4′,6′-triisopropyl-1,1′-biphenyl) [2-(2′-amino-1,1′-biphenyl)]palladium (II) (2nd Generation XPhos Precatalyst) or
   1,3-Bis(2,6-Di-3-pentylphenyl) imidazol-2-ylidene](3-chloropyridyl)dichloropalladium (II).

### Intermediate Compounds:

The present invention also discloses intermediate compounds useful in the preparation of compounds of general formula (I). In particular, the invention discloses compounds of general formula Ia in which Z is as defined for the compounds of general formula (I) supra and Y can be Br, I or Cl:

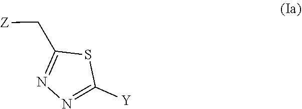

The present invention discloses further intermediate compounds useful in the preparation of compounds of formula (I). In particular, the present invention also discloses compounds of formula (Ib) in which in which A1, A2 and A3 are as defined for the compounds of general formula (I) supra:

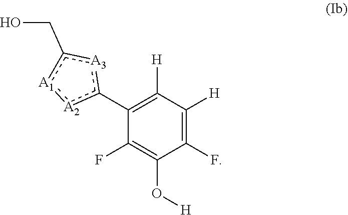

The following Table lists compounds 01-30 prepared according to the present invention.

| cpd # | Formula (I) |
|---|---|
| 01 | 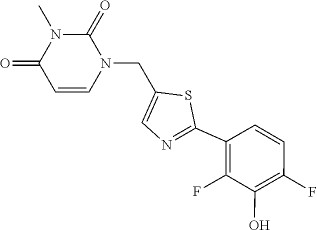 [SMILES: Cn1c(=O)ccn(Cc2cnc(-c3ccc(F)c(O)c3F)s2)c1=O] |
| 02 | 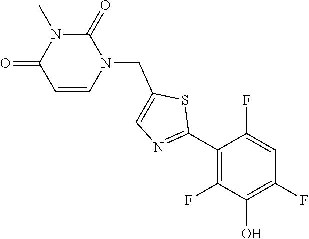 [SMILES: Cn1c(=O)ccn(Cc2cnc(-c3c(F)cc(F)c(O)c3F)s2)c1=O] |
| 03 | 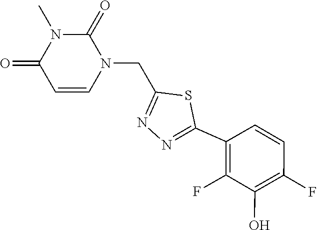 [SMILES: Cn1c(=O)ccn(Cc2nnc(-c3ccc(F)c(O)c3F)s2)c1=O] |
| 04 | 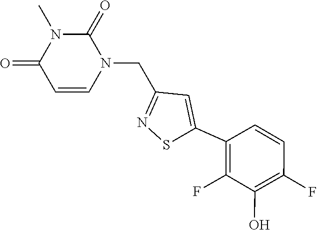 [SMILES: Cn1c(=O)ccn(Cc2cc(-c3ccc(F)c(O)c3F)sn2)c1=O] |
| 05 | 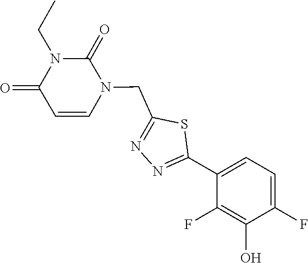 [SMILES: CCn1c(=O)ccn(Cc2nnc(-c3ccc(F)c(O)c3F)s2)c1=O] |
| 06 | 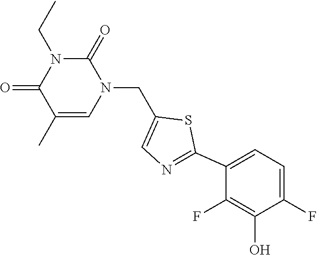 [SMILES: CCn1c(=O)c(C)cn(Cc2cnc(-c3ccc(F)c(O)c3F)s2)c1=O] |
| 07 | 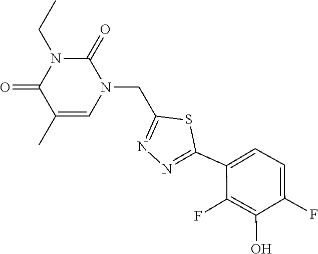 [SMILES: CCn1c(=O)c(C)cn(Cc2nnc(-c3ccc(F)c(O)c3F)s2)c1=O] |
| 08 | 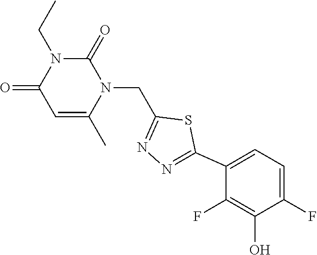 [SMILES: CCn1c(=O)cc(C)n(Cc2nnc(-c3ccc(F)c(O)c3F)s2)c1=O] |
| 09 | 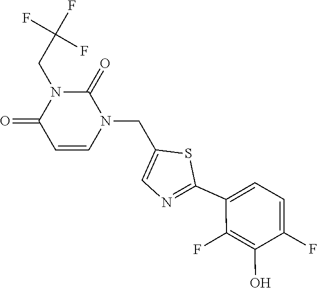 [SMILES: O=c1ccn(Cc2cnc(-c3ccc(F)c(O)c3F)s2)c(=O)n1CC(F)(F)F] |
| 10 | 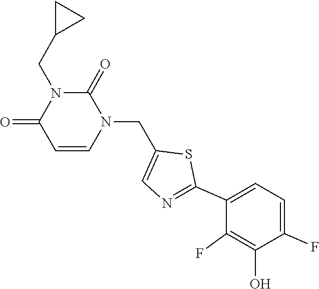 [SMILES: O=c1ccn(Cc2cnc(-c3ccc(F)c(O)c3F)s2)c(=O)n1CC1CC1] |
| 11 | 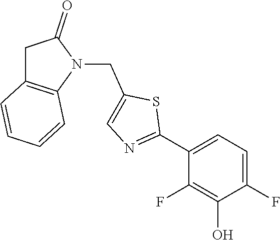 [SMILES: O=C1Cc2ccccc2N1Cc1cnc(-c2ccc(F)c(O)c2F)s1] |
| 12 | 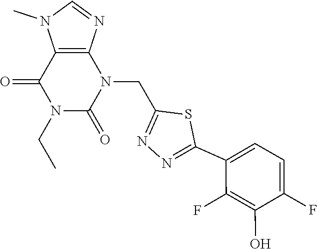 [SMILES: CCn1c(=O)c2c(ncn2C)n(Cc2nnc(-c3ccc(F)c(O)c3F)s2)c1=O] |
| 13 | 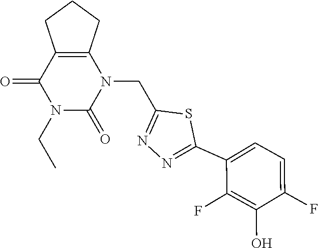 [SMILES: CCn1c(=O)c2c(n(Cc3nnc(-c4ccc(F)c(O)c4F)s3)c1=O)CCC2] |
| 14 | 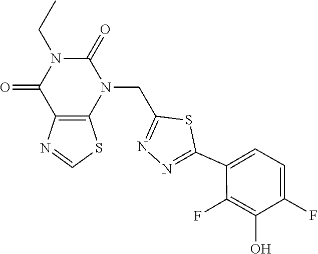 [SMILES: CCn1c(=O)c2ncsc2n(Cc2nnc(-c3ccc(F)c(O)c3F)s2)c1=O] |
| 15 | 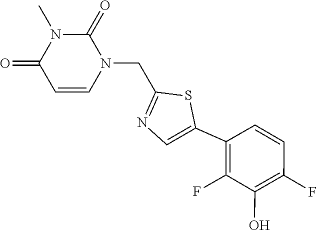 [SMILES: Cn1c(=O)ccn(Cc2ncc(-c3ccc(F)c(O)c3F)s2)c1=O] |
| 16 | 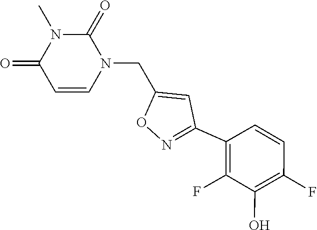 [SMILES: Cn1c(=O)ccn(Cc2cc(-c3ccc(F)c(O)c3F)no2)c1=O] |
| 17 | 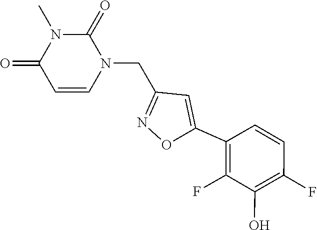 [SMILES: Cn1c(=O)ccn(Cc2cc(-c3ccc(F)c(O)c3F)on2)c1=O] |
| 18 | 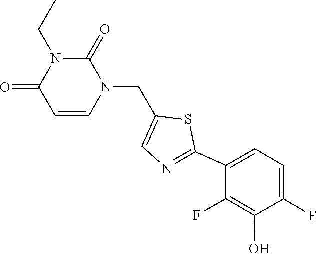 [SMILES: CCn1c(=O)ccn(Cc2cnc(-c3ccc(F)c(O)c3F)s2)c1=O] |
| 19 | 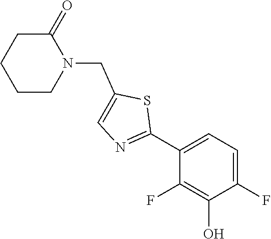 [SMILES: O=C1CCCCN1Cc1cnc(-c2ccc(F)c(O)c2F)s1] |
| 20 | 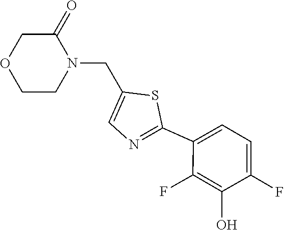 [SMILES: O=C1COCCN1Cc1cnc(-c2ccc(F)c(O)c2F)s1] |
| 21 | 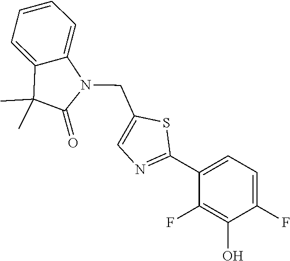 [SMILES: CC1(C)C(=O)N(Cc2cnc(-c3ccc(F)c(O)c3F)s2)c2ccccc21] |
| 22 | 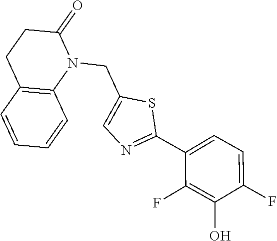 [SMILES: O=C1CCc2ccccc2N1Cc1cnc(-c2ccc(F)c(O)c2F)s1] |
| 23 | 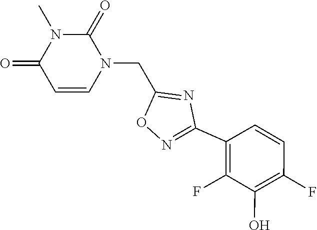 [SMILES: Cn1c(=O)ccn(Cc2nc(-c3ccc(F)c(O)c3F)no2)c1=O] |
| 24 | 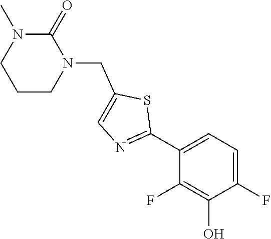 [SMILES: CN1CCCN(Cc2cnc(-c3ccc(F)c(O)c3F)s2)C1=O] |
| 25 | 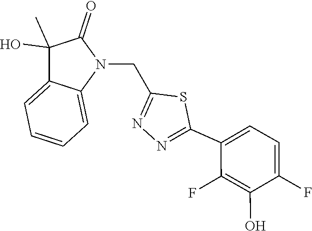 [SMILES: CC1(O)C(=O)N(Cc2nnc(-c3ccc(F)c(O)c3F)s2)c2ccccc21] |
| 26 | 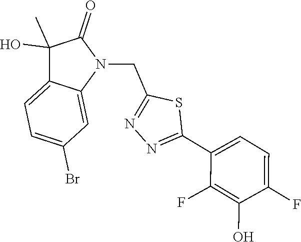 [SMILES: CC1(O)C(=O)N(Cc2nnc(-c3ccc(F)c(O)c3F)s2)c2cc(Br)ccc21] |
| 27 | 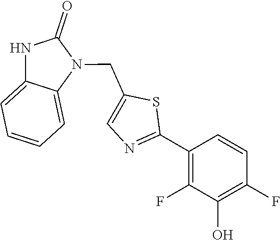 [SMILES: O=c1[nH]c2ccccc2n1Cc1cnc(-c2ccc(F)c(O)c2F)s1] |
| 28 | 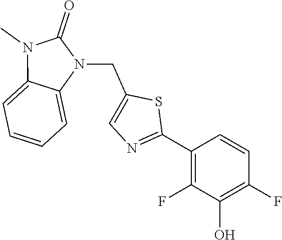 [SMILES: Cn1c(=O)n(Cc2cnc(-c3ccc(F)c(O)c3F)s2)c2ccccc21] |
| 29 | 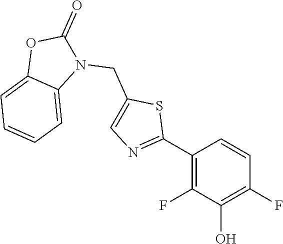 [SMILES: O=c1oc2ccccc2n1Cc1cnc(-c2ccc(F)c(O)c2F)s1] |
| 30 | 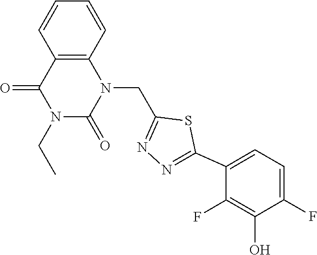 [SMILES: CCn1c(=O)c2ccccc2n(Cc2nnc(-c3ccc(F)c(O)c3F)s2)c1=O] |

Features and advantages of the present invention will become apparent from the following examples which illustrate the invention by way of example without restricting its scope.

ABBREVIATIONS

| | |
|---|---|
| ACN | acetonitrile |
| AcOH | acetic acid |
| aq. | aqueous |
| ° C. | degree Celsius |
| CDI | 1,1′-carbonyldiimidazole |
| conc. | concentrated |
| Cs₂CO₃ | cesium carbonate |
| DCM | dichloromethane |
| DIPEA | N,N-diisopropylethylamine |
| DMF | N,N-dimethylformamide |
| DTAD | di-tert.-butyl azodicarboxylate |
| EDC | ({[3-(dimethylamino)propyl]imino}methylidene)(ethyl)amine |
| ESI-MS | Electrospray ionization mass spectrometry |
| Et₂O | diethyl ether |
| EtOAc | ethyl acetate |
| EtOH | ethanol |
| h | hour |
| HCl | hydrochloric acid |
| HOBT | 1-hydroxybenzotriazole |
| HPLC | High performance liquid chromatography |
| HMDS | bis(trimethylsilyl)amine |
| K₂CO₃ | potassium carbonate |
| KOAc | potassium acetate |
| KOtBu | potassium tert.-butoxide |
| K₃PO₄ | tripotassium phosphate |
| L | liter |
| LiAlH₄ | lithium aluminium hydride |
| MeOH | methanol |
| MgSO₄ | magnesium sulfate |
| min | minute |
| mL | milliliter |
| n-BuLi | n-butyllithium |
| NaOAc | sodium acetate |
| Na₂SO₄ | sodium sulfate |
| NaOCl | sodium hypochlorite |
| NaOH | sodium hydroxide |
| PdCl₂(dtbpf) | [1,1′-bis(di-tert-butylphosphino)ferrocene]dichloropalladium(II |
| Pd(dppf)₂ × | 1,1′-bis(diphenyl-phosphino)-ferrocen-palladium(II) |
| CH₂Cl₂ | dichloride dichloromethane complex |
| Pd-Peppsi | [1,3-bis[2,6-bis(1-ethylpropyl)phenyl]-4,5-dichloro- |
| 2Me Ipent Cl | imidazol-2-yl]-dichloro-(2-methyl-1-pyridyl)palladium |
| RT | room temperature (about 20° C.) |
| sat. | saturated |
| t-BuOH | tert-butanol |
| TEA | triethylamine |
| TFA | trifluoroacetic acid |
| THF | tetrahydrofuran |
| TPP | triphenylphosphine |
| XPhos | 2-(dicyclohexylphosphanyl)-2′,4′,6′- |
| | tris(isopropyl)biphenyl |
| XPhos | chloro(2-dicyclohexylphosphino-2′,4′,6′-tri-i-propyl- |
| Pd G2 | 1,1′-biphenyl)(2′-amino-1,1′-biphenyl-2-yl) palladium (II) |

### EXAMPLES

### Example 1: Analytical HPLC methods

### 1.1 Method A

Analytical column: Waters; Sunfire C18_3.0×30 mm, 2.5 μm; column temperature: 60° Device: Agilent 1200 with DA- and MS-Detector

| | Vol % water | Vol % | Flow |
|---|---|---|---|
| time (min) | (incl. 0.1% TFA) | ACN | [mL/min] |
| 0.00 | 97.0 | 3.0 | 2.2 |
| 0.20 | 97.0 | 3.0 | 2.2 |
| 1.20 | 0.0 | 100.0 | 2.2 |
| 1.25 | 0.0 | 100.0 | 3.0 |
| 1.40 | 0.0 | 100.0 | 3.0 |

### 1.2 Method B

Analytical column: Waters; XBridge C18_3.0×30 mm, 2.5 μm; column temperature: 60° Device: Agilent 1200 with DA- and MS-Detector

| | Vol % water | Vol % | Flow |
|---|---|---|---|
| time (min) | (incl. 0.1% NH₄OH) | ACN | [mL/min] |
| 0.00 | 97.0 | 3.0 | 2.2 |
| 0.20 | 97.0 | 3.0 | 2.2 |
| 1.20 | 0.0 | 100.0 | 2.2 |
| 1.25 | 0.0 | 100.0 | 3.0 |
| 1.40 | 0.0 | 100.0 | 3.0 |

### 1.3 Method C

Analytical column: Waters; Sunfire C18_2.1×30 mm, 2.5 μm; column temperature: 60° Device: Waters Acquity with DA- and MS-Detector

| | Vol % water | Vol % | Flow |
|---|---|---|---|
| time (min) | (incl. 0.1% TFA) | ACN | [mL/min] |
| 0.00 | 99.0 | 1.0 | 1.5 |
| 0.02 | 99.0 | 1.0 | 1.5 |
| 1.00 | 0.0 | 100.0 | 1.5 |
| 1.10 | 0.0 | 100.0 | 1.5 |

### 1.4 Method D

Analytical column: Sunfire C18_3.0×30 mm, 2.5 μm; column temperature: 60° Device: Waters Acquity, QDa Detector

| time | Vol % water | Vol % ACN | Flow |
|---|---|---|---|
| (min) | (incl. 0.1% TFA) | (incl. 0.08% TFA) | [mL/min] |
| 0.00 | 95.0 | 5.0 | 1.5 |
| 1.30 | 0.0 | 100.0 | 1.5 |
| 1.50 | 0.0 | 100.0 | 1.5 |
| 1.60 | 95.0 | 5.0 | 1.5 |

### 1.5 Method E

Analytical column: Sunfire C18_3.0×30 mm, 2.5 μm; column temperature: 60° Device: Waters Acquity, QDa Detector

| time | Vol % water | Vol % ACN | Flow |
|---|---|---|---|
| (min) | (incl. 0.1% TFA) | (incl. 0.08% TFA) | [mL/min] |
| 0.00 | 95.0 | 5.0 | 1.5 |
| 1.30 | 0.0 | 100.0 | 1.5 |
| 1.50 | 0.0 | 100.0 | 1.5 |
| 1.60 | 95.0 | 5.0 | 1.5 |

### Example 2: Preparation of Intermediates

### 2.1 Intermediate I

Intermediate I.1 (general procedure)

1-[(2-Bromo-1,3-thiazol-5-yl)methyl]-3-methyl-1,2,3,4-tetrahydropyrimidine-2,4-dione

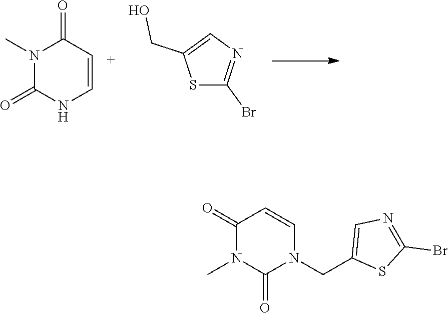

3-Methyl-1,2,3,4-tetrahydropyrimidine-2,4-dione (3.00 g; 23.79 mmol) and 3-bromothiazol-5-methanol (5.08 g; 26.17 mmol) were dissolved in THF (50 mL) and DMF (20 mL). TPP (polymer bound 3 mmol/g; 10.50 g; 30.92 mmol) was added and the reaction mixture cooled in an ice bath. DTAD (7.12 g; 30.92 mmol) was added. After stirring over night at RT, the reaction mixture was filtered and the THF was evaporated. The residue was poured on water and extracted several times with EtOAc. The combined organic layers were washed with brine, dried over Na₂SO₄, filtered off and concentrated under reduced pressure. The residue was purified by column chromatography (silica gel; DCM/MeOH, 100/0 up to 95/5) to provide the product.
C₉H₈BrN₃O₂S (M=302.2 g/mol)
ESI-MS: 302 [M+H]⁺
Rt(HPLC): 0.67 min (method A)
Yield: 4.03 g; 56%
¹H NMR (400 MHZ, DMSO-d₆) δ ppm: 3.16 (s, 3H), 5.10 (s, 2H), 5.76 (d, J=7.86 Hz, 1H), 7.73 (s, 1H), 7.84 (d, J=7.86 Hz, 1H).

The intermediate compounds I.2 and I.3 shown in the table below were prepared using procedures analogous to those described for intermediate I. 1 using appropriate starting materials. As is appreciated by those skilled in the art, these analogous examples may involve variations in general reaction conditions.

| | | | Reaction conditions |
|---|---|---|---|
| | | | (deviation from |
| Int. | Starting materials | Structure | general procedure) |
| I.2 | 3-methyl-1,2,3,4- tetrahydro- pyrimidine- 2,4-dione and (5-chloro- 1,3-thiazol-2- yl)methanol | 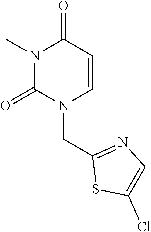 [SMILES: Cn1c(=O)ccn(Cc2ncc(Cl)s2)c1=O] | residue dissolved in DMF; left for 2 days; precipitate filtered off |
| I.3 | 5-methyl- 1,2,3,4- tetrahydro- pyrimidine- 2,4-dione and (2-bromo- 1,3-thiazol-5- yl)methanol | 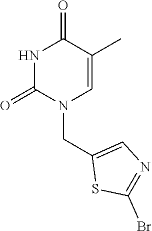 [SMILES: Cc1cn(Cc2cnc(Br)s2)c(=O)[nH]c1=O] | unbound TPP |

| | | HPLC | |
|---|---|---|---|
| | | retention | |
| | | time | |
| Int. | ESI-MS | (method) | 1H NMR |
| I.2 | 258 | 0.78 min | ¹H NMR (400 MHz, DMSO-d₆) δ ppm: |
| | | (B) | 3.16 (s, 3 H), 5.21 (s, 2 H), 5.78 (d, J = |
| | | | 7.86 Hz, 1 H), 7.79 (s, 1 H), 7.82 (d, |
| | | | J = 7.86 Hz, 1 H). |
| I.3 | 302 | 0.74 min | ¹H NMR (400 MHz, DMSO-d₆) δ ppm: |
| | | (A) | 1.75 (d, J = 1.01 Hz, 3 H), 5.00 (s, 2 H), |
| | | | 7.66 (d, J = 1.14 Hz, 1 H), 7.71 (s, |
| | | | 1 H), 11.40 (s, 1 H). |

### Intermediate I.4

1-[(5-bromo-1,3,4-thiadiazol-2-yl)methyl]-3-methyl-1,2,3,4-tetrahydropyrimidine-2,4-dione

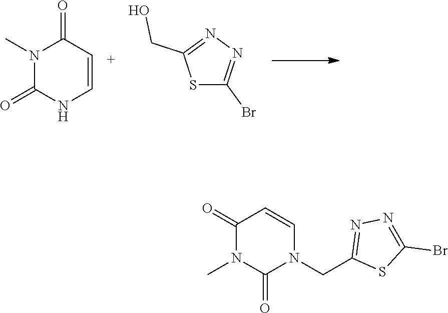

To a stirred mixture of (5-bromo-1,3,4-thiadiazol-2-yl) methanol (0.20 g; 1.03 mmol) and TEA (0.17 mL; 1.23 mmol) in DCM (5 mL) methanesulfonyl chloride (0.10 mL; 1.23 mmol) was added dropwise. After stirring for 1 h at RT, another methanesulfonyl chloride (0.04 mL) was added and stirred for a further hour. The reaction mixture was diluted with DCM and water. The organic layer was separated through a phase separator cartridge and evaporated. The residue was taken up in DMF (3 mL), 3-methyl-1,2,3,4-tetrahydropyrimidine-2,4-dione (0.10 g; 0.79 mmol) and K₂CO₃ (0.28 g; 2.03 mmol) were added. The reaction mixture was attired at RT overnight, filtered and purified by column chromatography (reversed phase; Sunfire C18; water/ACN/TFA) to provide the product.
C₈H₇BrN₄O₂S (M=303.1 g/mol)
ESI-MS: 303/305 (Br) [M+H]⁺
Rₜ (HPLC): 0.71 min (method A)
Yield: 0.14 g; 58%

### Intermediate I.5

1-[(5-Bromo-1,2-thiazol-3-yl)methyl]-3-methyl-1,2,3,4-tetrahydropyrimidine-2,4-dione

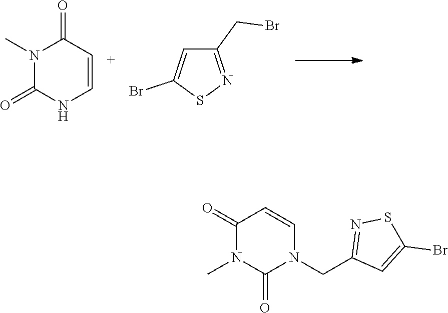

3-Methyl-1,2,3,4-tetrahydropyrimidine-2,4-dione (0.04 g; 0.32 mmol), 5-bromo-3-(bromomethyl)-1,2-thiazole (0.09 mL; 0.35 mmol) and K₂CO₃ (0.10 g; 0.70 mmol) in dry DMF (2 mL) were stirred at RT for 2 h. The reaction mixture was partitioned between EtOAc and water. The organic layer was washed with brine and water, dried over Na₂SO₄, filtered and concentrated under reduced pressure.
C₉H₈BrN₃O₂S (M=302.2 g/mol)
ESI-MS: 302 [M+H]⁺
Rₜ (HPLC): 0.71 min (method A)
Yield: 0.09 g; 97%
¹H NMR (400 MHZ, DMSO-d₆) δ ppm: 3.15 (s, 3H), 5.07 (s, 2H), 5.76 (d, J=7.86 Hz, 1 H), 7.56 (s, 1H), 7.77 (d, J=7.86 Hz, 1H).

### 2.2 Intermediate II

[3-(Benzyloxy)-2,4-difluorophenyl]boronic acid

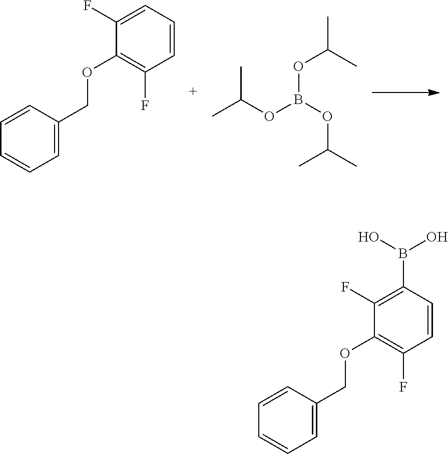 [SMILES: Fc1cccc(F)c1OCc1ccccc1 ; CC(C)OB(OC(C)C)OC(C)C ; OB(O)c1ccc(F)c(OCc2ccccc2)c1F]

2-(Benzyloxy)-1,3-difluorobenzene (0.50 g; 2.27 mmol) in THF (10 mL) was cooled to −78° C. n-BuLi (2.5 mol/L in THF; 1.36 mL; 3.63 mmol) was added dropwise and stirred for 50 minutes in the cold. Triisopropyl borate (0.73 mL; 3.63 mmol) was added dropwise stirred for further 10 min at −78° C. and then further 30 minutes without cooling. The reaction was quenched with HCl (aq. solution; 4 mol/L; 5 mL) and stirred for 10 minutes. The mixture was poured on water and extracted several times with EtOAc. The combined organic layers were dried over MgSO₄, filtered off and concentrated under reduced pressure. The residue was purified by column chromatography (silica gel; DCM/MeOH with few AcOH, 100/0 up to 96/4) to provide the product.
C₁₃H₁₁BF₂O₃ (M=264.0 g/mol)
ESI-MS: 263 [M−H]⁻
Rₜ (HPLC): 0.75 min (method B)
Yield: 0.25 g; 42%
¹H NMR (400 MHZ, DMSO-d₆) δ ppm: 5.12 (s, 2H), 7.05 (ddd, J=10.27, 8.62, 1.39 Hz, 1 H), 7.25 (dt, J=8.40, 6.57 Hz, 1H), 7.30-7.56 (m, 5H), 8.23 (br s, 2H).

### 2.3 Intermediate III

### Intermediate III. 1

(2,4-Difluoro-3-hydroxyphenyl) boronic acid

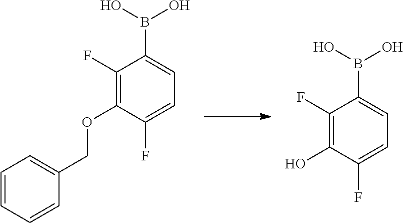 [SMILES: OB(O)c1ccc(F)c(OCc2ccccc2)c1F ; OB(O)c1ccc(F)c(O)c1F]

Intermediate II.1 (0.10 g; 0.38 mmol) in a mixture of THF and MeOH (each 3 mL) was hydrogenated in a parr apparatus using Pd/C 10% (0.02 g) at RT for 3 h. The reaction mixture was concentrated under reduced pressure. The residue was purified by column chromatography (reversed phase; Sunfire C18; water/ACN/TFA) to provide the product.
C₆H₅BF₂O₃ (M=173.9 g/mol)
ESI-MS: 173 [M−H]⁻
Rₜ (HPLC): 0.07 min (method B)
Yield 0.05 g; 77%
¹H NMR (400 MHZ, DMSO-d₆) δ ppm: 6.90-7.00 (m, 2H), 8.11 (br s, 2H), 9.81 (br s, 1 H).

### 2.4 Intermediate IV

Intermediate IV.1 (general procedure)

2,4-Difluoro-3-[(4-methoxyphenyl) methoxy]benzaldehyde

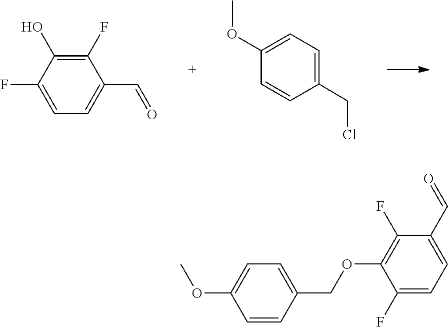

1-(Chloromethyl)-4-methoxybenzene (0.19 mL; 1.39 mmol) added to a mixture of 2,4-difluoro-3-hydroxybenzaldehyde (0.20 g; 1.27 mmol) and K₂CO₃ (0.27 g; 1.94 mmol) in ACN (5 mL). After stirring at 60° C. for 3 h, the reaction mixture was filtered and diluted with DMF/water/TFA and purified by column chromatography (reversed phase; Sunfire C18; water/ACN/TFA). ACN was evaporated in the desired fractions and the product was partitioned between DCM and water. The organic layer was dried over MgSO₄, filtered and concentrated under reduced pressure.
C₁₅H₁₂F₂O₃ (M=278.3 g/mol)
ESI-MS: 277 [M−H]⁻
Rₜ (HPLC): 1.12 min (method A)
Yield: 0.22 g; 63%
¹H NMR (400 MHZ, DMSO-d₆) δ ppm: 3.75 (s, 3H), 5.14 (s, 2H), 6.93 (d, J=8.74 Hz, 2 H), 7.26-7.39 (m, 3H), 7.59 (ddd, J=8.81, 7.41, 6.08 Hz, 1H), 10.11 (s, 1H).

The intermediate compounds IV.2 and IV.4 shown in the table below were prepared using procedures analogous to those described for intermediate IV.1 using appropriate starting materials. As is appreciated by those skilled in the art, these analogous examples may involve variations in general reaction conditions.

| | | | Reaction conditions (deviation |
|---|---|---|---|
| Int. | Starting materials | Structure | from general procedure) |
| IV.2 | Intermediate VII.1 and 4-methoxybenzyl chloride | 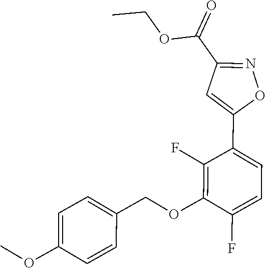 [SMILES: CCOC(=O)c1cc(-c2ccc(F)c(OCc3ccc(OC)cc3)c2F)on1] | stirred for 30 min at 60° C. and for 1 h at 70° C. |
| IV.3 | 3-bromo-2,6- difluorophenol and 4-methoxybenzyl chloride | 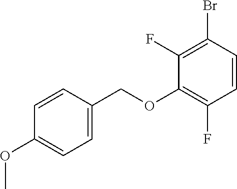 [SMILES: COc1ccc(COc2c(F)ccc(Br)c2F)cc1] | stirred for 2 h at 70° C. |
| IV.4 | Intermediate VII.2 and 4-methoxybenzyl chloride | 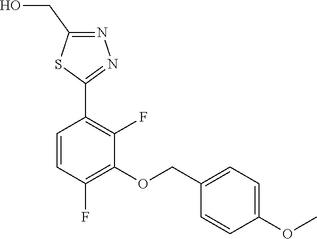 [SMILES: COc1ccc(COc2c(F)ccc(-c3nnc(CO)s3)c2F)cc1] | stirred for 6.5 h at 80° C.; purification by evaporating and treating with water to create precipitate |

| | ESI- | HPLC retention | |
|---|---|---|---|
| Int. | MS | time (method) | ¹H NMR |
| IV.2 | 390 | 1.20 min | |
| | | (A) | |
| IV.3 | | | |
| IV.4 | 365 | 1.02 | ¹H NMR (400 MHz, DMSO-d₆) |
| | | (A) | δ ppm: 3.75 (s, 3 H), |
| | | | 4.94 (s, 2 H), 5.17 (s, 2 H), |
| | | | 6.30 (br s, 1 H), 6.94 (d, |
| | | | J = 8.62 Hz, 2 H), 7.29-7.40 |
| | | | (m, 3 H), 7.92 (ddd, |
| | | | J = 8.87, 7.73, 5.96 Hz, 1 H). |

### 2.5 Intermediate V

### Intermediate V.1

({2,4-Difluoro-3-[(4-methoxyphenyl) methoxy]phenyl}methylidene) hydroxylamine

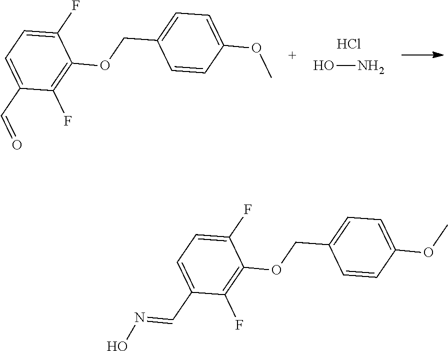

Intermediate IV.1 (0.22 g; 0.79 mmol) and NaOAc (0.08 g; 1.03 mmol) in MeOH (8 mL) and water (3 mL) were stirred at RT. Hydroxylamine hydrochloride (0.04 mL; 1.03 mmol) was added and the reaction mixture heated up to 80° C. for 1.5 h. The reaction mixture was concentrated under reduced pressure and taken up in water/EtOAc. The aqueous layer was extracted several times with EtOAc. The combined organic layers were dried over MgSO₄, filtered and concentrated under reduced pressure.
C₁₅H₁₃F₂NO₃ (M=293.3 g/mol)
ESI-MS: 294 [M+H]⁺
Rₜ (HPLC): 1.06 min (method A)
Yield: 0.25 g; quantitative
¹H NMR (400 MHZ, DMSO-d₆) δ ppm: 3.75 (s, 3H), 5.10 (s, 2H), 6.90-6.96 (m, 2H), 7.10-7.19 (m, 1H), 7.31-7.37 (m, 2H), 7.42 (ddd, J=8.81, 7.79, 6.08 Hz, 1H), 8.14 (s, 1H), 11.59 (s, 1H).

### 2.6 Intermediate VI

### Intermediate VI.1

1-[(3-{2,4-Difluoro-3-[(4-methoxyphenyl) methoxy]phenyl}-1,2-oxazol-5-yl)methyl]-3-methyl-1,2,3,4-tetrahydropyrimidine-2,4-dione

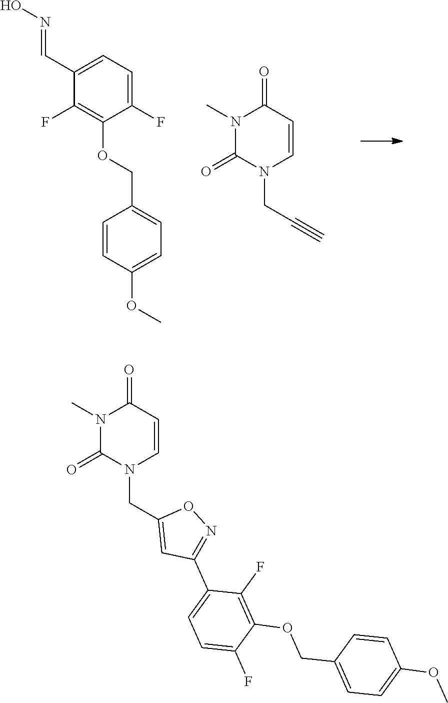 [SMILES: COc1ccc(COc2c(F)ccc(/C=N/O)c2F)cc1 ; C#CCn1ccc(=O)n(C)c1=O ; COc1ccc(COc2c(F)ccc(-c3cc(Cn4ccc(=O)n(C)c4=O)on3)c2F)cc1]

Intermediate V.1 (0.10 g; 0.34 mmol), 3-methyl-1-(prop-2-yn-1-yl)-1,2,3,4-tetrahydropyrimidine-2,4-dione (0.06 g; 0.34 mmol) and TEA (0.01 mL; 0.03 mmol) in t-BuOH (4 mL) and water (4 mL) were stirred at RT. NaOCl (0.45 mL; 0.58 mmol) was added. After stirring for 2.5 h at RT, EtOAc and water were added. The organic layer was separated, dried over MgSO₄, filtered and concentrated under reduced pressure. The residue was purified by column chromatography (reversed phase; Sunfire C18; water/ACN/TFA) to provide the product.
C₂₃H₁₉F₂N₃O₅ (M=455.4 g/mol)
ESI-MS: 456 [M+H]⁺
Rₜ (HPLC): 1.07 min (method A)
Yield: 0.02 g; 12%
¹H NMR (400 MHZ, DMSO-d₆) δ ppm: 3.17 (s, 3H), 3.75 (s, 3H), 5.14 (s, 2H), 5.20 (s, 2 H), 5.81 (d, J=7.86 Hz, 1H), 6.89 (d, J=2.66 Hz, 1H), 6.93 (d, J=8.74 Hz, 2H), 7.26 (td, J=9.57, 1.77 Hz, 1H), 7.35 (d, J=8.62 Hz, 2H), 7.56 (ddd, J=8.90, 7.83, 5.96 Hz, 1 H), 7.86 (d, J=7.98 Hz, 1H).
   Intermediate VI.2 (general procedure)

1-[(5-{2,4-Difluoro-3-[(4-methoxyphenyl) methoxy]phenyl}-1,2-oxazol-3-yl)methyl]-3-methyl-1,2,3,4-tetrahydropyrimidine-2,4-dione

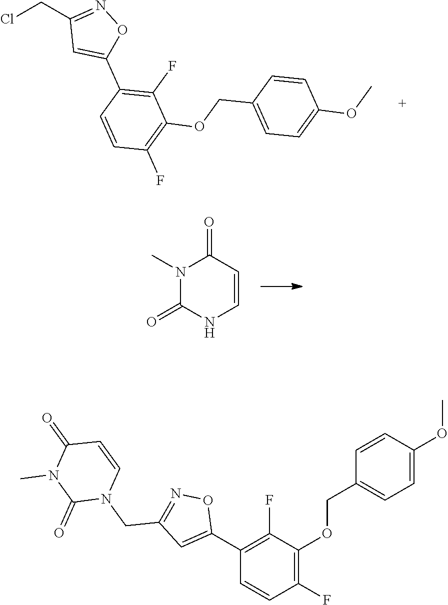

Intermediate IX.1 (0.10 g; 0.03 mmol), 3-methyl-1,2,3,4-tetrahydropyrimidine-2,4-dione (0.01 g; 0.05 mmol) and K₂CO₃ (0.01 g; 0.08 mmol) in DMF (2 mL) were stirred for 1 h at RT and for 30 min at 50° C. The reaction mixture was filtered and purified by column chromatography (reversed phase; Sunfire C18; water/ACN/TFA) to provide the product.
C₂₃H₁₉F₂N₃O₅ (M=455.4 g/mol)
ESI-MS: 456 [M+H]⁺
Rₜ (HPLC): 1.07 min (method A)
Yield 0.01 g; 53%

The following intermediate compound VI.3 below was prepared using procedures analogous to those described for the intermediate VI.2 using appropriate starting materials. As is appreciated by those skilled in the art, these analogous examples may involve variations in general reaction conditions.

| Int. | Starting materials | Structure |
|---|---|---|
| VI.3 | Intermediate IX.2 and 1,2,3,4- tetrahydropyrimidine-2,4- dione | 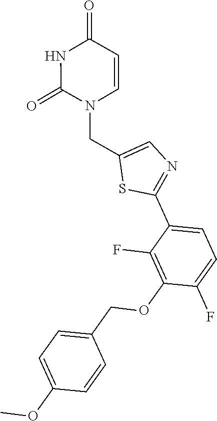 [SMILES: COc1ccc(COc2c(F)ccc(-c3ncc(Cn4ccc(=O)[nH]c4=O)s3)c2F)cc1] |

| | | HPLC | |
|---|---|---|---|
| | | retention | |
| | ESI- | time | |
| Int. | MS | (method) | ¹H NMR |
| VI.3 | 458 | 1.01 min | ¹H NMR (400 MHz, DMSO-d₆) |
| | | (A) | δ ppm: 3.73 (s, 3 H), 5.11 |
| | | | (s, 2 H), 5.24 (d, J = 3.42 Hz, |
| | | | 2 H), 5.86 (d, J = 7.86 Hz, 1 |
| | | | H), 6.91 (d, J = 8.36 Hz, 2 H), |
| | | | 7.18-7.27 (m, 1 H), 7.33 |
| | | | (d, J = 8.49 Hz, 2 H), 7.84 (tt, |
| | | | J = 8.46, 5.67 Hz, 1 H), 7.90- |
| | | | 7.99 (m, 1 H), 8.03 (d, J = |
| | | | 2.03 Hz, 1 H). |

### 2.7 Intermediate VII

### Intermediate VII.1

Ethyl 5-(2,4-difluoro-3-hydroxyphenyl)-1,2-oxazole-3-carboxylate

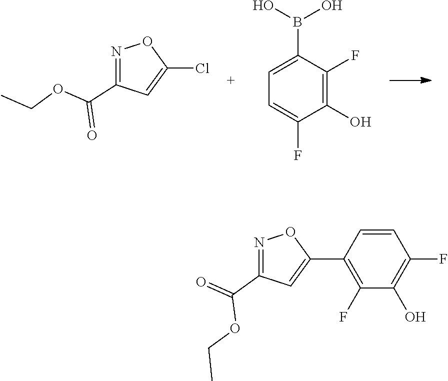 [SMILES: CCOC(=O)c1cc(Cl)on1 ; OB(O)c1ccc(F)c(O)c1F ; CCOC(=O)c1cc(-c2ccc(F)c(O)c2F)on1]

Ethyl 5-chloro-1,2-oxazole-3-carboxylate (0.15 g; 0.85 mmol), intermediate III.1 (0.22 g; 1.28 mmol) and K₃PO₄ (0.36 g; 1.71 mmol) were dissolved in dioxane (3 mL). The reaction mixture was flushed with nitrogen. Pd-Peppsi 2Me-Ipent Cl (0.04 g; 0.04 mmol) was added. After stirring for 20 min at 65° C., the reaction mixture was diluted with DMF/water and purified by column chromatography (reversed phase; Sunfire C18; water/ACN/TFA) to provide the product.
C₁₂H₉F₂NO₄ (M=269.2 g/mol)
ESI-MS: 270 [M+H]⁺
Rₜ (HPLC): 0.98 min (method A)
Yield: 0.03 g; 11%
¹H NMR (400 MHZ, DMSO-d₆) δ ppm: 1.35 (t, J=7.16 Hz, 3H), 4.40 (q, J=7.10 Hz, 2 H), 7.16 (d, J=2.91 Hz, 1H), 7.25 (td, J=9.57, 1.77 Hz, 1H), 7.43 (ddd, J=8.84, 7.64, 5.70 Hz, 1H), 10.77 (br s, 1H).

### Intermediate VII.2

2,6-Difluoro-3-[5-(hydroxymethyl)-1,3,4-thiadiazol-2-yl]phenol

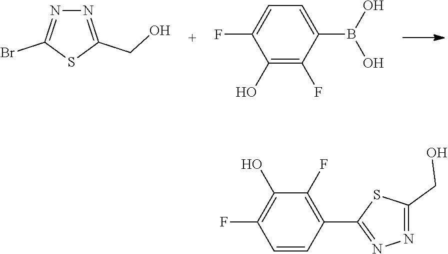 [SMILES: OCc1nnc(Br)s1 ; OB(O)c1ccc(F)c(O)c1F ; OCc1nnc(-c2ccc(F)c(O)c2F)s1]

The reaction was performed under nitrogen atmosphere. (5-Bromo-1,3,4-thiadiazol-2-yl) methanol (4.00 g; 20.51 mmol), intermediate III.1 (4.60 g; 24.61 mmol) and Na₂CO₃ (5.43 g; 51.27 mmol) were dissolved in EtOH (50 mL) and water (10 mL). Pd-Peppsi 2Me-Ipent Cl (0.86 g; 1.03 mmol) was added and the mixture stirred at 80° overnight. The reaction mixture was filtered and the filtrate concentrated under reduced pressure. The residue was taken up in water and the resulting precipitate filtered off to provide the product.
C₉H₆F₂N₂O₂S (M=244.2 g/mol)
ESI-MS: 245 [M+H]⁺
Rₜ (HPLC): 0.72 min (method A)
Yield: 2.66 g; 53%
¹H NMR (400 MHZ, DMSO-d₆) δ ppm: 4.93 (d, J=4.69 Hz, 2H), 6.27 (br t, J=5.39 Hz, 1 H), 7.19-7.34 (m, 1H), 7.65 (ddd, J=8.84, 7.57, 5.89 Hz, 1H), 10.75 (s, 1H).

### 2.8 Intermediate VIII

### Intermediate VIII.1

(5-{2,4-Difluoro-3-[(4-methoxyphenyl) methoxy]phenyl}-1,2-oxazol-3-yl) methanol

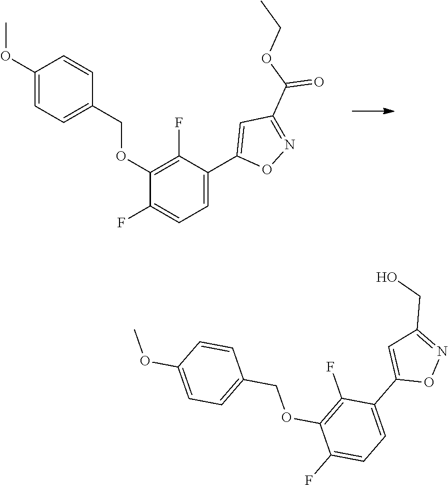 [SMILES: CCOC(=O)c1cc(-c2ccc(F)c(OCc3ccc(OC)cc3)c2F)on1 ; COc1ccc(COc2c(F)ccc(-c3cc(CO)no3)c2F)cc1]

Intermediate IV.2 (0.03 g; 0.07 mmol) in THF (3 mL) was cooled to −20° C. LiAlH₄ (solution in THF; 1 mol/L; 0.05 mL; 0.05 mol/L) was added dropwise and stirred for 30 min in the cold. Two drops of water and NaOH (aq. solution; 4 mol/L) were added and stirred for further 20 min at RT. The reaction mixture was filtered, diluted with watere and purified by column chromatography (reversed phase; Sunfire C18; water/ACN/TFA) to provide the product.
C₁₈H₁₅F₂NO₄ (M=347.3 g/mol)
ESI-MS: 348 [M+H]⁺
Rₜ (HPLC): 1.04 min (method A)
Yield: 0.02 g; 70%
¹H NMR (400 MHZ, DMSO-d₆) δ ppm: 3.75 (s, 3H), 4.57 (d, J=6.08 Hz, 2H), 5.16 (s, 2 H), 5.55 (t, J=6.02 Hz, 1H), 6.82 (d, J=3.55 Hz, 1H), 6.93 (d, J=8.74 Hz, 2H), 7.30 (ddd, J=10.36, 8.90, 1.90 Hz, 1H), 7.36 (d, J=8.62 Hz, 2H), 7.63 (ddd, J=8.90, 7.83, 5.83 Hz, 1H).

### 2.9 Intermediate IX

### Intermediate IX.1

3-(Chloromethyl)-5-{2,4-difluoro-3-[(4-methoxyphenyl) methoxy]phenyl}-1,2-oxazole

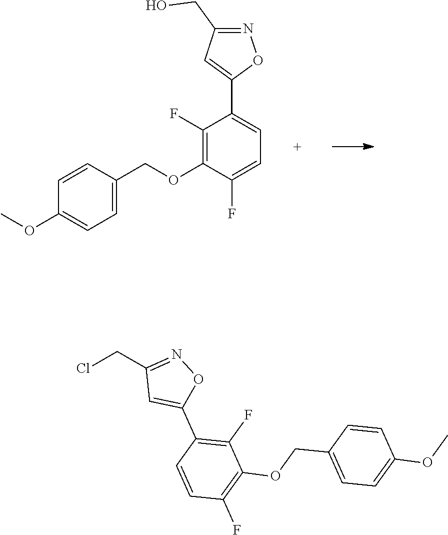 [SMILES: COc1ccc(COc2c(F)ccc(-c3cc(CO)no3)c2F)cc1 ; COc1ccc(COc2c(F)ccc(-c3cc(CCl)no3)c2F)cc1]

Intermediate VIII.1 (0.02 g; 0.024 mmol), TPP (0.01 g; 0.04 mmol) and carbon tetrachloride (0.02 mL; 0.22 mmol) in ACN (2 mL) were stirred for 2.67 h at 80° C. The reaction mixture was concentrated under reduced pressure. The residue was used in the further reaction as crude product.
C₁₈H₁₄ClF₂NO₃ (M=365.8 g/mol)
ESI-MS: 366 [M+H]⁺
Rₜ (HPLC): 1.20 min (method A)
Yield: 0.01 g; 70% (determined by HPLC-MS)

### Intermediate IX.2 (General Procedure)

5-(Chloromethyl)-2-{2,4-difluoro-3-[(4-methoxyphenyl) methoxy]phenyl}-1,3-thiazole

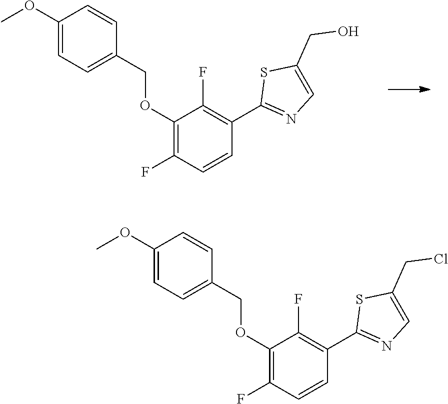 [SMILES: COc1ccc(COc2c(F)ccc(-c3ncc(CO)s3)c2F)cc1 ; COc1ccc(COc2c(F)ccc(-c3ncc(CCl)s3)c2F)cc1]

Intermediate XI.1 (0.99 g; 2.72 mmol) and TEA (0.60 mL; 4.33 mmol) in DCM (10 mL) were stirred in an ice bath. Methanesulfonyl chloride (0.32 mL; 4.13 mmol) was added dropwise. After stirring for 5 min in the cold, the reaction mixture was stirred at RT overnight. The reaction mixture was diluted with additional DCM and washed with diluted citric acid. The organic layer was separated with a phase separator cartridge and evaporated.
C₁₈H₁₄ClF₂NO₂S (M=381.8 g/mol)
ESI-MS: 382 [M+H]⁺
Rₜ (HPLC): 1.22 min (method A)
Yield: 1.01 g; 97%
¹H NMR (400 MHZ, DMSO-d₆) δ ppm: 3.75 (s, 3H), 5.16 (s, 2H), 5.18 (s, 2H), 6.93 (d, J=8.74 Hz, 2H), 7.28 (td, J=9.63, 1.90 Hz, 1H), 7.36 (d, J=8.62 Hz, 2H), 7.89 (ddd, J=9.00, 8.05, 6.02 Hz, 1H), 8.03 (d, J=2.28 Hz, 1H).

The following intermediate compound IX.3 was prepared using procedures analogous to those described for the intermediate IX.2 using appropriate starting materials. As is appreciated by those skilled in the art, these analogous examples may involve variations in general reaction conditions.

| | | | Reaction | | | |
|---|---|---|---|---|---|---|
| | | | conditions | | | |
| | | | (deviation | | HPLC | |
| | | | from | | retention | |
| | Starting | | general | ESI- | time | |
| Int. | materials | Structure | procedure) | MS | (method) | ¹H NMR |
| IX.3 | Intermediate IV.4 | 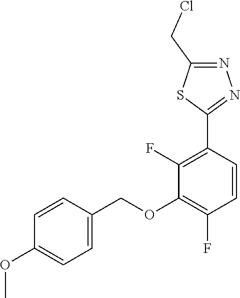 | | 383 | 1.10 (A) | |

### 2.10 Intermediate X

### Intermediate X.1

2,4-Difluoro-N′,3-dihydroxybenzene-1-carboximidamide

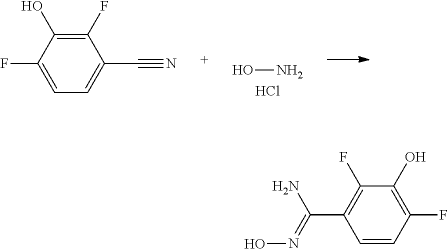

2,4-Difluoro-3-hydroxybenzonitrile (0.03 g; 0.20 mmol) in EtOH (0.5 mL) was stirred at RT. Hydroxylamine hydrochloride (0.03 g; 0.40 mmol) and TEA (0.06 mL; 0.04 mmol) in EtOH (1 mL) was added dropwise. After stirring for 20 h at RT the reaction mixture was concentrated under reduced pressure and further used as crude product.

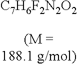

### 2.11 Intermediate XI

### Intermediate XI.1

(2-{2,4-Difluoro-3-[(4-methoxyphenyl) methoxy]phenyl}-1,3-thiazol-5-yl) methanol

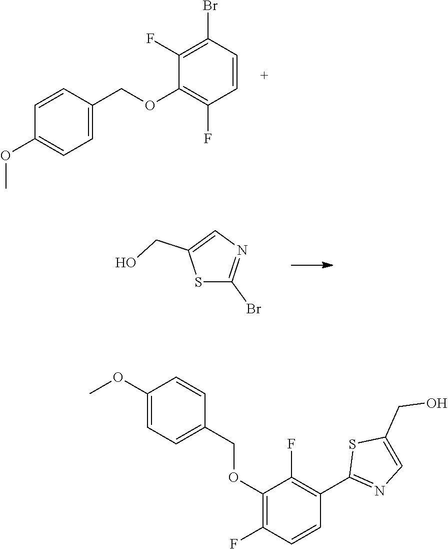 [SMILES: COc1ccc(COc2c(F)ccc(Br)c2F)cc1 ; OCc1cnc(Br)s1 ; COc1ccc(COc2c(F)ccc(-c3ncc(CO)s3)c2F)cc1]

Intermediate IV.3 (3.00 g; 9.11 mmol), bis(pinacolato)diboron (3.50 g; 13.78 mmol), KOAc (2.30 g; 23.44 mmol) and Pd(dppf)₂ x CH₂Cl₂ (0.75 g; 0.92 mmol) in dioxane (50 mL) were stirred overnight at 90° C. Additional bis(pinacolato)diboron (0.70 g; 2.76 mmol) was added and the reaction mixture stirred for further 2 h at 100° C. After cooling to RT, Pd(dppf)₂ x CH₂Cl₂ (0.38 g; 0.46 mmol), (2-bromo-1,3-thiazol-5-yl) methanol (1.80 g; 9.28 mmol) and Na₂CO₃ (aq. solution; 2 mol/L; 15.00 mL; 30.00 mmol) were added and the reaction mixture was stirred for 6 h at reflux. The mixture was cooled to RT, diluted with EtOAc and filtered through Celite®. The filtrate was concentrated under reduced pressure. The residue was partitioned between EtOAc and water. The organic layer was separated, dried over Na₂SO₄, filtered and concentrated under reduced pressure. The residue was purified by column chromatography (silica gel; cyclohexane/EtOAC 2:1 to 1:1). Et₂O was added to the evaporated fractions, stirred and filtered off. The precipitate was dried in air.
C₁₈H₁₅F₂NO₃S (M=363.4 g/mol)
ESI-MS: 364 [M+H]⁺
Rₜ (HPLC): 1.07 min (method A)
Yield: 1.10 g; 30%
¹H NMR (400 MHZ, DMSO-d₆) δ ppm: 3.75 (s, 3H), 4.75 (d, J=5.70 Hz, 2H), 5.16 (s, 2 H), 5.64 (t, J=5.77 Hz, 1H), 6.93 (d, J=8.74 Hz, 2H), 7.26 (td, J=9.63, 1.77 Hz, 1H), 7.36 (d, J=8.62 Hz, 2H), 7.79-7.83 (m, 1H), 7.86 (ddd, J=8.93, 8.11, 6.02 Hz, 1H).

### 2.12 Intermediate XII

Intermediate XII.1 (general procedure)

1-[(2-{2,4-Difluoro-3-[(4-methoxyphenyl) methoxy]phenyl}-1,3-thiazol-5-yl)methyl]-3-ethyl-1,2,3,4-tetrahydropyrimidine-2,4-dione

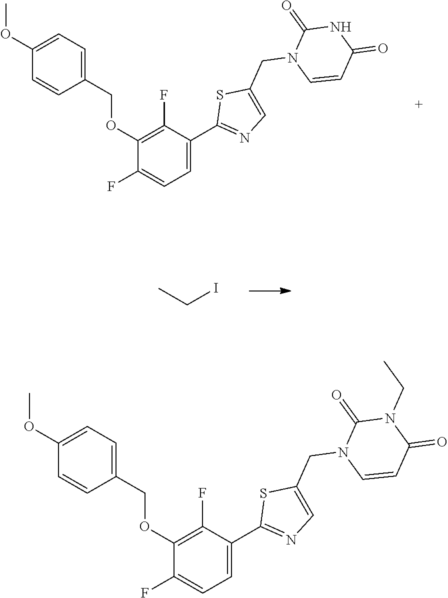

Intermediate VI.3 (0.03 g; 0.05 mmol), iodoethane (0.01 mL; 0.16 mmol) and K₂CO₃ (0.03 g; 0.16 mmol) in DMF (3 mL) were stirred at 50° C. for 2 h. The reaction mixture was diluted with water, filtered and purified by column chromatography (reversed phase; Sunfire C18; water/ACN/TFA) to provide the product.
C₂₄H₂₁F₂N₃O₄S (M=485.5 g/mol)
Rₜ (HPLC): 1.12 min (method A)
Yield: 0.02 g; 64%

The following intermediate compounds XII.2 to XII.7 below were prepared using procedures analogous to those described for the intermediate XII. 1 using appropriate starting materials. As is appreciated by those skilled in the art, these analogous examples may involve variations in general reaction conditions.

| | | | Reaction conditions |
|---|---|---|---|
| | | | (deviation from |
| Int. | Starting materials | Structure | general procedure) |
| XII.2 | Intermediate XIII.1 and iodoethane | 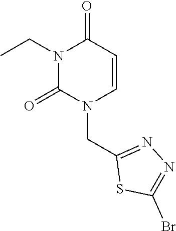 [SMILES: CCn1c(=O)ccn(Cc2nnc(Br)s2)c1=O] | stirred at 70° C.; purification by extraction from water with DCM |
| XII.3 | Intermediate I.3 and iodoethane | 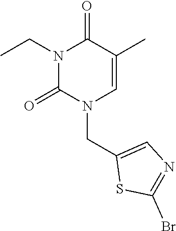 [SMILES: CCn1c(=O)c(C)cn(Cc2cnc(Br)s2)c1=O] | |
| XII.4 | Intermediate XIII.2 and iodoethane | 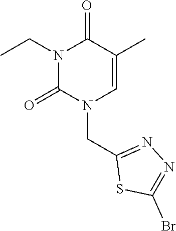 [SMILES: CCn1c(=O)c(C)cn(Cc2nnc(Br)s2)c1=O] | purification by performing precipitate using water |
| XII.5 | Intermediate XV.1 and 1,1,1-trifluoro-2- iodoethane | 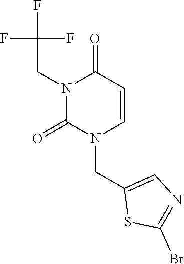 [SMILES: O=c1ccn(Cc2cnc(Br)s2)c(=O)n1CC(F)(F)F] | |
| XII.6 | Intermediate XV.1 and (iodomethyl) cyclopropane) | 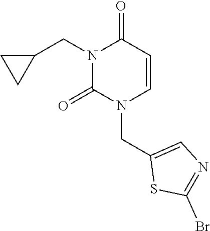 [SMILES: O=c1ccn(Cc2cnc(Br)s2)c(=O)n1CC1CC1] | stepwise heating up from 50° to 100° C. during 3.5 h |
| XII.7 | Intermediate XIII.3 and iodoethane | 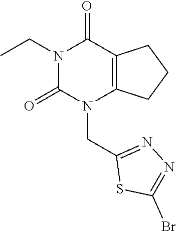 [SMILES: CCn1c(=O)c2c(n(Cc3nnc(Br)s3)c1=O)CCC2] | purification by extraction between water and DCM |

| | | HPLC | |
|---|---|---|---|
| | | retention | |
| | | time | |
| Int. | ESI-MS | (method) | ¹H NMR |
| XII.2 | 331 | | |
| XII.3 | 330 | 0.93 min | |
| | | (A) | |
| XII.4 | 331 | | ¹H NMR (400 MHz, DMSO-d₆) |
| | | | δ ppm: 1.08 (t, J = 7.03 Hz, 3 H), |
| | | | 1.82 (d, J = 1.14 Hz, 3 H), 3.84 |
| | | | (q, J = 7.10 Hz, 2 H), 5.34 (s, 2 H), |
| | | | 7.73 (q, J = 1.14 Hz, 1 H). |
| XII.5 | 370 | 0.95 | |
| | | (A) | |
| XII.6 | 342 | 0.96 | |
| | | (A) | |
| XII.7 | | 0.91 | |
| | | (A) | |

### 2.13 Intermediate XIII

Intermediate XIII.1 (general procedure)

1-[(5-Bromo-1,3,4-thiadiazol-2-yl)methyl]-1,2,3,4-tetrahydropyrimidine-2,4-dione

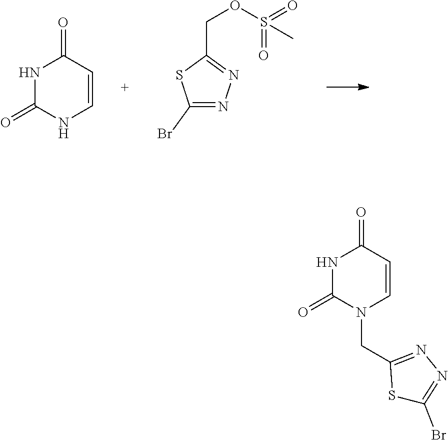

The reaction was performed under an argon atmosphere. 1,2,3,4-Tetrahydropyrimidine-2,4-dion (0.50 g; 4.46 mmol) and (E)-(trimethylsilyl N-(trimethylsilyl) ethanimidate (2.75 mL; 11.25 mmol) in ACN (15 mL) were stirred for 5 h at RT. (5-Bromo-1,3,4-thiadiazol-2-yl)methyl methanesulfonat (1.40 g; 5.13 mmol) and tetrabutylazanium iodide (0.17 g; 0.46 mmol) were added. After stirring for 6 h at 80° C. and overnight at RT, 30 mL of water was slowly added. The resulting precipitate was filtered off and washed with water, ACN and Et₂O.
C₇H₅BrN₄O₂S (M=289.1 g/mol)
Yield: 0.29 g; 23%

The following intermediate compounds XIII.2 to XIII.4 below were prepared using procedures analogous to those described for the intermediate XIII. 1 using appropriate starting materials. As is appreciated by those skilled in the art, Zanalogous examples may involve variations in general reaction conditions.

| Int. | Starting materials | Structure |
|---|---|---|
| XIII.2 | 5-methyl-1,2,3,4- tetrahydropyrimidine-2,4-dione and (5-bromo-1,3,4-thiadiazol-2-yl) methyl methanesulfonat | 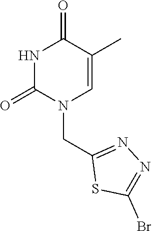 [SMILES: Cc1cn(Cc2nnc(Br)s2)c(=O)[nH]c1=O] |
| XIII.3 | 1H,2H,3H,4H,5H,6H,7H- cyclopenta[d]pyrimidine-2,4- dione and (5-bromo-1,3,4-thiadiazol-2-yl) methyl methanesulfonat | 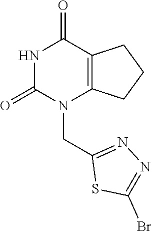 [SMILES: O=c1[nH]c(=O)n(Cc2nnc(Br)s2)c2c1CCC2] |
| XIII.4 | Intermediate IX.3 and 1,2,3,4-tetrahydroquinazoline-2,4-dione | 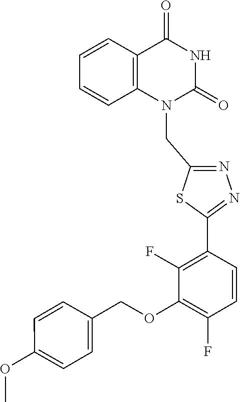 [SMILES: COc1ccc(COc2c(F)ccc(-c3nnc(Cn4c(=O)[nH]c(=O)c5ccccc54)s3)c2F)cc1] |

| | | HPLC | |
|---|---|---|---|
| | | retention time | |
| Int. | ESI-MS | (method) | ¹H NMR |
| XIII.2 | 303/305 | 0.67 | ¹H NMR (400 MHz, DMSO-d₆) |
| | | (A) | δ ppm: 1.76 (d, J = 0.76 |
| | | | Hz, 3 H), 5.28 (s, 2 H), 7.67 |
| | | | (q, J = 1.01 Hz, 1 H), 11.47 |
| | | | (s, 1 H). |
| XIII.3 | 329 | 0.67 | ¹H NMR (400 MHz, DMSO-d₆) |
| | | (A) | δ ppm: 1.99 (br quin, |
| | | | J = 7.54 Hz, 2 H), 2.53 (br t, |
| | | | J = 7.48 Hz, 2 H), 2.93 (br |
| | | | t, J = 7.60 Hz, 2 H), 5.30 |
| | | | (s, 2 H), 11.29 (s, 1 H). |
| XIII.4 | 509 | 1.00 | |
| | | (A) | |

### 2.14 Intermediate XIV

### Intermediate XIV.1

1-[(5-Bromo-1,3,4-thiadiazol-2-yl)methyl]-6-methyl-1,2,3,4-tetrahydropyrimidine-2,4-dione

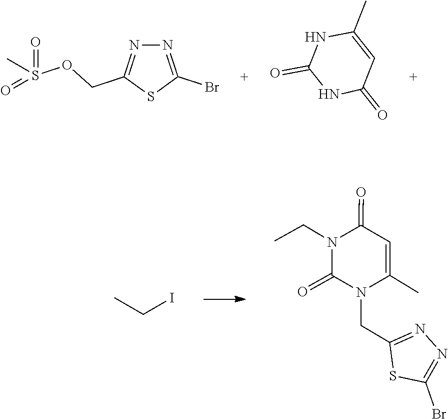

(5-Bromo-1,3,4-thiadiazol-2-yl)methyl methanesulfonat (0.20 g; 0.73 mmol) and 6-methyl-1,2,3,4-tetrahydropyrimidine-2,4-dione (0.18 g; 1.46 mmol) were dissolved in DMF (5 mL), K₂CO₃ (0.25 g; 1.83 mmol) is added. After stirring for 2 h at RT, iodoethane (0.15 mL; 1.83 mmol) was added and stirred for another hour at 50° C. The reaction mixture was purified by column chromatography (reversed phase; Sunfire C18; water/ACN/TFA) to provide the product.
C₁₀H₁₁BrN₄O₂S (M=331.2 g/mol)
ESI-MS: 331 [M+H]⁺
Rₜ (HPLC): 0.83 min (method A)
Yield: 0.03 g; 14%
¹H NMR (400 MHZ, DMSO-d₆) δ ppm: 1.08 (t, J=7.03 Hz, 3H), 2.31 (d, J=0.63 Hz, 3 H), 3.82 (q, J=6.97 Hz, 2H), 5.42 (s, 2H), 5.71 (q, J=0.63 Hz, 1H).

### 2.15 Intermediate XV

### Intermediate XV.1

1-[(2-Bromo-1,3-thiazol-5-yl)methyl]-1,2,3,4-tetrahydropyrimidine-2,4-dione

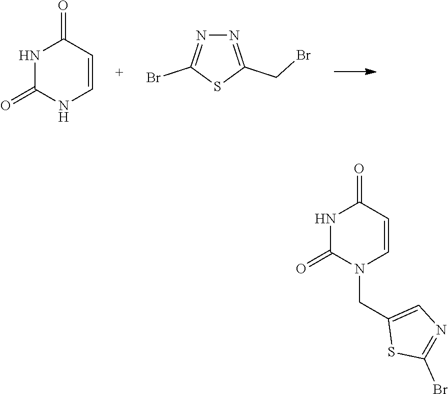 [SMILES: O=c1cc[nH]c(=O)[nH]1 ; BrCc1nnc(Br)s1 ; O=c1ccn(Cc2cnc(Br)s2)c(=O)[nH]1]

The reaction was performed under a nitrogen atmosphere. 1,2,3,4-Tetrahydropyrimidine-2,4-dione (1.35 g; 12.04 mmol), HMDS (2.76 mL; 13.25 mmol) and chlorotrimethylsilane (0.76 mL; 6.02 mmol) in dry ACN (10 mL) were stirred for 5 h at 140° C. and then concentrated under reduced pressure. Half of the residue was taken up in dry ACN (22 mL), 2-bromo-5-(bromomethyl)-1,3-thiazole (2.90 g; 11.29 mmol) in ACN (13 mL) was added dropwise at 0° C. After complete addition, the mixture was heated up to 80° C. and stirred overnight. After cooling to RT, the reaction mixture was diluted with NaHCO₃ (9% aq. solution) and extracted several times from EtOAc. The organic layer was separated, dried over Na₂SO₄, filtered and concentrated under reduced pressure. The residue was treated with Et₂O and filtered.
C₈H₆BrN₃O₂S (M=288.1 g/mol)
ESI-MS: 288/290 [M+H]⁺
Rₜ (HPLC): 0.67 min (method A)
Yield: 2.66 g; 77%
¹H NMR (400 MHz, DMSO-d₆) δ ppm: 5.04 (s, 2H), 5.62 (d, J=7.86 Hz, 1H), 7.71 (s, 1 H), 7.78 (d, J=7.86 Hz, 1H), 11.41 (br s, 1H).

### 2.16 Intermediate XVI

Intermediate XVI.1 (general procedure)

1-[(2-{2,4-Difluoro-3-[(4-methoxyphenyl) methoxy]phenyl}-1,3-thiazol-5-yl)methyl]piperidin-2-one

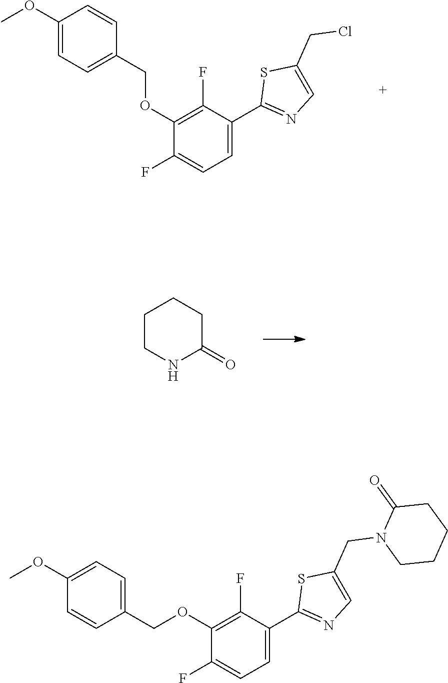 [SMILES: COc1ccc(COc2c(F)ccc(-c3ncc(CCl)s3)c2F)cc1 ; O=C1CCCCN1 ; COc1ccc(COc2c(F)ccc(-c3ncc(CN4CCCCC4=O)s3)c2F)cc1]

Piperidin-2-one (0.03 g; 0.26 mmol) and sodium hydride (0.01 g; 0.26 mmol) in DMF (2 mL) were stirred at RT for 10 min. Intermediate IX.2 (0.04 g; 0.10 mmol) was added. After stirring for 2 h, the mixture was diluted with water and MeOH, and purified by column chromatography (reversed phase; Sunfire C18; water/ACN/TFA) to provide the desired product.
C₂₃H₂₂F₂N₂O₃S (M=444.5 g/mol)
ESI-MS: 445 [M+H]⁺
Rₜ (HPLC): 1.11 min (method A)
Yield: 0.02 g; 52%

The following intermediate compounds XVI.2 to XVI.4 below were prepared using procedures analogous to those described for the intermediate XVI.1 using appropriate starting materials. As is appreciated by those skilled in the art, these analogous examples may involve variations in general reaction conditions.

| | | | Reaction |
|---|---|---|---|
| | | | conditions |
| | | | (deviation |
| | | | from general |
| Int. | Starting materials | Structure | procedure) |
| XVI.2 | Intermediate IX.2 and morpholin-3-one | 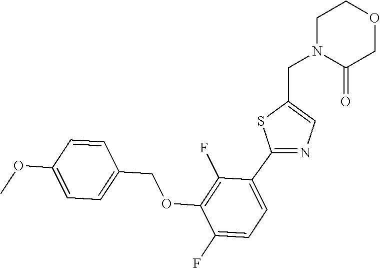 [SMILES: COc1ccc(COc2c(F)ccc(-c3ncc(CN4CCOCC4=O)s3)c2F)cc1] | |
| XVI.3 | Intermediate IX.2 and 3,3-dimethyl-2,3- dihydro-1H-indol- 2-one | 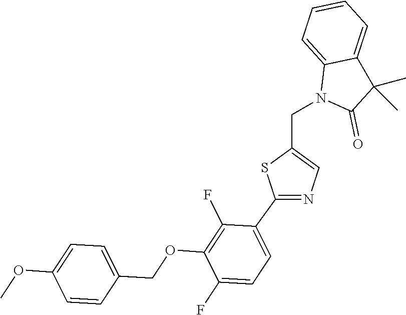 | |
| XVI.4 | Intermediate IX.2 and 1,2,3,4- tetrahydroquinolin- 2-one | 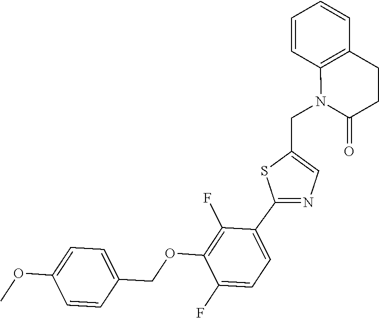 [SMILES: COc1ccc(COc2c(F)ccc(-c3ncc(CN4C(=O)CCc5ccccc54)s3)c2F)cc1] | |

| | ESI- | HPLC retention |
|---|---|---|
| Int. | MS | time (method) |
| XVI.2 | 447 | 1.06 |
| | | (A) |
| XVI.3 | 507 | 1.24 |
| | | (A) |
| XVI.4 | 493 | 1.21 |
| | | (A) |

### 2.17 Intermediate XVII

### Intermediate XVII.1

tert-Butyl N-(3-{[(2-{2,4-difluoro-3-[(4-methoxyphenyl) methoxy]phenyl}-1,3-thiazol-5-yl)methyl]amino}propyl)-N-methylcarbamate

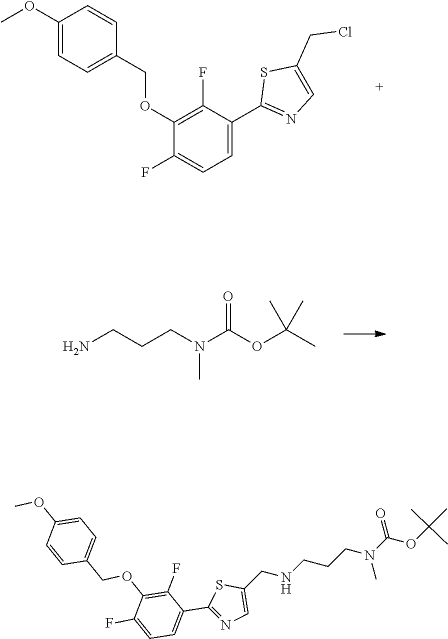 [SMILES: COc1ccc(COc2c(F)ccc(-c3ncc(CCl)s3)c2F)cc1 ; CN(CCCN)C(=O)OC(C)(C)C ; COc1ccc(COc2c(F)ccc(-c3ncc(CNCCCN(C)C(=O)OC(C)(C)C)s3)c2F)cc1]

Intermediate IX.2 (0.03 g; 0.07 mmol); tert-butyl N-(3-aminopropyl)-N-methylcarbamate (0.01 g; 0.07 mmol) and K₂CO₃ (0.02 g; 0.13 mmol) in ACN (3.84 mL) were stirred at 80° C. for 2 h. Additional three equivalents of amine and base were added and stirred for further 1.4 h at 80° C. The reaction mixture was diluted with water and MeOH, and purified by column chromatography (reversed phase; Sunfire C18; water/ACN/TFA) to provide the desired product.
C₂₇H₃₃F₂N₃O₄S (M=533.6 g/mol)
ESI-MS: 534 [M+H]⁺
Rₜ (HPLC): 0.94 min (method A)
Yield: 0.03 g; 86%

### 2.18 Intermediate XVIII

Intermediate XVIII.1 (general procedure)

1-[(2-Bromo-1,3-thiazol-5-yl)methyl]-2,3-dihydro-1H-indol-2-one

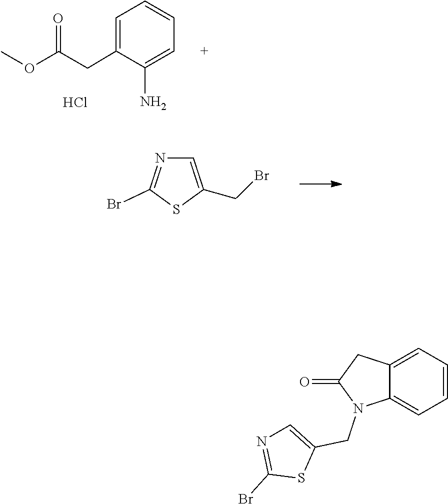 [SMILES: COC(=O)Cc1ccccc1N ; Cl ; BrCc1cnc(Br)s1 ; O=C1Cc2ccccc2N1Cc1cnc(Br)s1]

Methyl 2-(2-aminophenyl)acetate hydrochloride (0.10 g; 0.50 mmol); 2-bromo-5-(bromomethyl)-1,3-thiazol (0.14 g; 0.50 mmol) and DIPA (0.21 mL; 1.24 mmol) in DCM (5 mL) were stirred at RT. The reaction mixture was concentrated under reduced pressure. The residue was taken up in DMF and water, and purified by column chromatography (reversed phase; Sunfire C18; water/ACN/TFA) to provide the desired product.
C₁₂H₉BrN₂OS (M=309.2 g/mol)
ESI-MS: 309 [M+H]⁺
Rₜ (HPLC): 0.96 min (method A)
Yield: 0.02 g; 14%
¹H NMR (400 MHZ, DMSO-d₆) δ ppm: 3.61 (s, 2H), 5.09 (s, 2H), 7.02 (td, J=7.48, 1.01 Hz, 1H), 7.16 (d, J=7.73 Hz, 1H), 7.23-7.30 (m, 2H), 7.78 (s, 1H).

### 2.19 Intermediate XIX

Intermediate XIX.1 (general procedure)

1-Ethyl-7-methyl-2,3,6,7-tetrahydro-1H-purine-2,6-dione

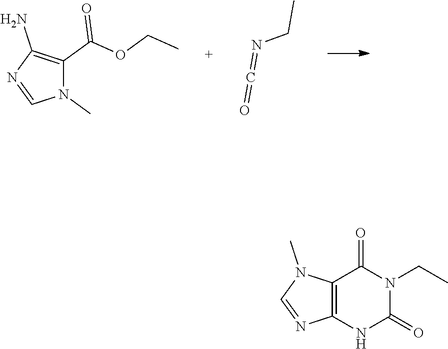

Ethyl 4-amino-1-methyl-1H-imidazole-5-carboxylate (bromomethyl)-1,3-thiazol (0.20 g; 1.15 mmol) and isocyanatoethane (0.16 mL; 1.95 mmol) in pyridine (1 mL) were stirred for 2 h at 70° C. KOtBu (0.20 g; 1.72 mmol) is added. After stirring over night at 70° C., the reaction mixture was quenched with MeOH, concentrated under reduced pressure, taken up in DMF and water, and purified by column chromatography (reversed phase; Sunfire C18; water/ACN/TFA) to provide the desired product.
C₈H₁₀N₄O₂ (M=194.2 g/mol)
ESI-MS: 195 [M+H]⁺
Rₜ (HPLC): 0.28 min (method C)
Yield: 0.10 g; 43%
¹H NMR (400 MHZ, DMSO-d₆) δ ppm: 1.10 (t, J=6.97 Hz, 3H), 3.81-3.90 (m, 5H), 7.90 (s, 1H), 11.77 (s, 1H).

The following intermediate compound was prepared using procedures analogous to those described for intermediate XIX.1 using appropriate starting materials. As is appreciated by those skilled in the art, these analogous examples may involve variations in general reaction conditions.

| | Starting | | Reaction conditions (deviation from |
|---|---|---|---|
| Int. | materials | Structure | general procedure) |
| XIX.2 | ethyl 5-amino- 1,3-thiazole-4- carboxylate and isocyanatoethane | 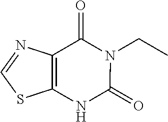 | Heating up to 105° C. and used base for ring closure was NH₄OH (5% aq. solution) |

| | | HPLC | |
|---|---|---|---|
| | | retention | |
| | ESI- | time | |
| Int. | MS | (method) | ¹H NMR |
| XIX.2 | 198 | 0.25 | ¹H NMR (400 MHz, DMSO-d₆) |
| | | (C) | δ ppm: 1.13 (t, J = 7.03 Hz, 3 H), |
| | | | 3.89 (q, J = 6.97 Hz, 2 H), |
| | | | 8.73 (s, 1 H), 12.23 (br s, 1 H). |

### 2.20 Intermediate XX

### Intermediate XX.1 (General Procedure)

3-[(5-Bromo-1,3,4-thiadiazol-2-yl)methyl]-1-ethyl-7-methyl-2,3,6,7-tetrahydro-1H-purine-2,6-dione

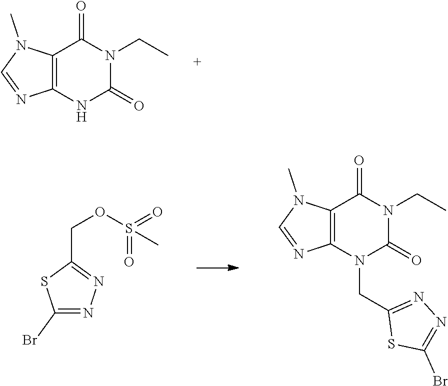 [SMILES: CCn1c(=O)[nH]c2ncn(C)c2c1=O ; CS(=O)(=O)OCc1nnc(Br)s1 ; CCn1c(=O)c2c(ncn2C)n(Cc2nnc(Br)s2)c1=O]

Intermediate XIX.1 (0.10 g; 0.51 mmol), (5-bromo-1,3,4-thiadiazol-2-yl)methyl methanesulfonat (0.27 g; 0.99 mmol) and K₂CO₃ (0.21 g; 1.54 mmol) in DMF (6 mL) were stirred at 80° C. for 2.5 h. The reaction mixture was concentrated under reduced pressure, taken up in water and DCM. The organic layer was washed with water and brine, dried over Na₂SO₄, filtered and concentrated under reduced pressure to give the desired product, which was used in the next next without further purification.
C₁₁H₁₁BrN₆O₂S (M=371.2 g/mol)
ESI-MS: 371 [M+H]⁺
Rₜ (HPLC): 0.75 min (method A)
Yield: 0.20 g; 89%

The following intermediate compound was prepared using procedures analogous to those described for intermediate XX.1 using appropriate starting materials. As is appreciated by those skilled in the art, these analogous examples may involve variations in general reaction conditions.

| | | | | HPLC | |
|---|---|---|---|---|---|
| | | | | retention | Reaction conditions |
| | Starting | | | time | (deviation from |
| Int. | materials | Structure | ESI-MS | (method) | general procedure) |
| XX.2 | Intermediate XIX.2 and (5-bromo-1,3,4- thiadiazol-2- yl)methyl | 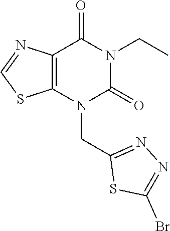 [SMILES: CCn1c(=O)c2ncsc2n(Cc2nnc(Br)s2)c1=O] | 374/376 | 0.73 (A) | purification by column chromatography (reversed phase; XBridge; water(ACN/ NH₄OH) |

### Example 3: Preparation of Example Compounds

### 3.1 General Procedure

1-{[2-(2,4-Difluoro-3-hydroxyphenyl)-1,3-thiazol-5-yl]methyl}-3-methyl-1,2,3,4-tetrahydropyrimidine-2,4-dione (example compound 1)

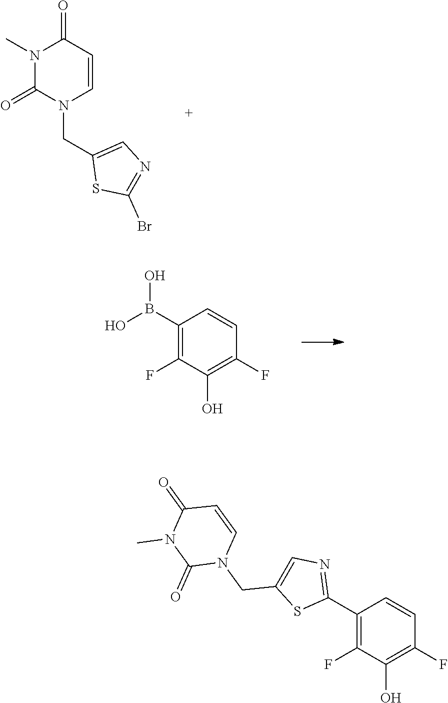 [SMILES: Cn1c(=O)ccn(Cc2cnc(Br)s2)c1=O ; OB(O)c1ccc(F)c(O)c1F ; Cn1c(=O)ccn(Cc2cnc(-c3ccc(F)c(O)c3F)s2)c1=O]

Pd-Peppsi 2Me-Ipent C1 (0.06 g; 0.07 mmol) was added to a stirred mixture of Intermediate I.1 (0.40 g; 1.32 mmol), Intermediate III.1 (0.35 g; 2.01 mmol) and Cs₂CO₃ (1.10 g; 3.38 mmol) in water (1 mL) and EtOH (4 mL). After stirring for 1 h at 100° C., the reaction mixture was filtered through Celite® and washed with EtOH. The filtrate was concentrated under reduced pressure. The residue was taken up in DCN and water. The organic layer was evaporated and the resulting precipitate was filtered off, washed with water, MeOH and Et₂O to give the desired product.
C₁₅H₁₁F₂N₃O₃S (M=351.3 g/mol)
ESI-MS: 352 [M+H]⁺
Rₜ (HPLC): 0.84 min (method A)
Yield: 0.43 g; 92%
¹H NMR (400 MHZ, DMSO-d₆) δ ppm: 3.17 (s, 3H), 5.20 (s, 2H), 5.77 (d, J=7.98 Hz, 1 H), 7.18 (td, J=9.57, 1.90 Hz, 1H), 7.60 (ddd, J=8.93, 7.92, 5.96 Hz, 1H), 7.90 (d, J=7.86 Hz, 1H), 8.00 (d, J=2.28 Hz, 1H), 10.51-10.71 (m, 1H).

### 3.2 Example Compounds (Ex.) 2-14

The following compounds were prepared using procedures analogous to those described under 3.1 above using appropriate starting materials. As is appreciated by those skilled in the art, these analogous examples may involve variations in general reaction conditions.

| Ex. | Structure |
|---|---|
| 2 | 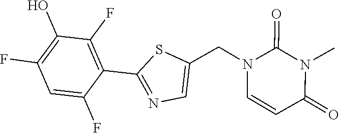 [SMILES: Cn1c(=O)ccn(Cc2cnc(-c3c(F)cc(F)c(O)c3F)s2)c1=O] |
| 3 | 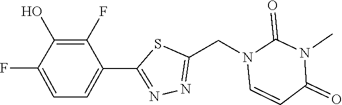 [SMILES: Cn1c(=O)ccn(Cc2nnc(-c3ccc(F)c(O)c3F)s2)c1=O] |
| 4 | 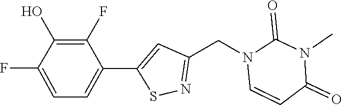 [SMILES: Cn1c(=O)ccn(Cc2cc(-c3ccc(F)c(O)c3F)sn2)c1=O] |
| 5 | 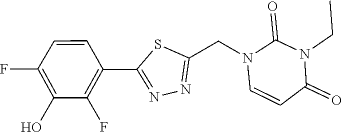 [SMILES: CCn1c(=O)ccn(Cc2nnc(-c3ccc(F)c(O)c3F)s2)c1=O] |
| 6 | 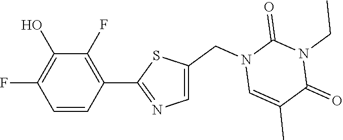 [SMILES: CCn1c(=O)c(C)cn(Cc2cnc(-c3ccc(F)c(O)c3F)s2)c1=O] |
| 7 | 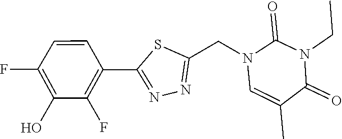 [SMILES: CCn1c(=O)c(C)cn(Cc2nnc(-c3ccc(F)c(O)c3F)s2)c1=O] |
| 8 | 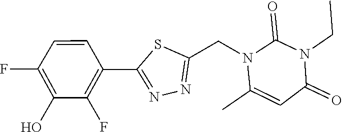 [SMILES: CCn1c(=O)cc(C)n(Cc2nnc(-c3ccc(F)c(O)c3F)s2)c1=O] |
| 9 | 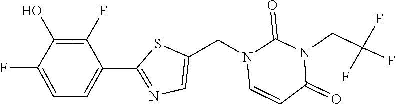 [SMILES: O=c1ccn(Cc2cnc(-c3ccc(F)c(O)c3F)s2)c(=O)n1CC(F)(F)F] |
| 8 | 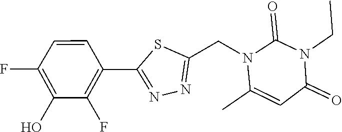 [SMILES: CCn1c(=O)cc(C)n(Cc2nnc(-c3ccc(F)c(O)c3F)s2)c1=O] |
| 9 | 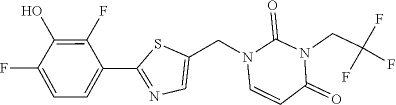 [SMILES: O=c1ccn(Cc2cnc(-c3ccc(F)c(O)c3F)s2)c(=O)n1CC(F)(F)F] |
| 10 | 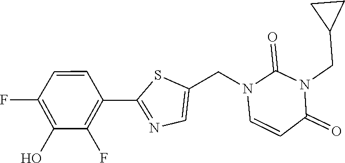 [SMILES: O=c1ccn(Cc2cnc(-c3ccc(F)c(O)c3F)s2)c(=O)n1CC1CC1] |
| 11 | 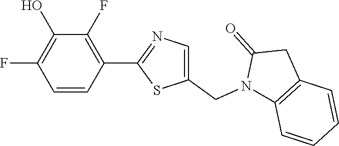 [SMILES: O=C1Cc2ccccc2N1Cc1cnc(-c2ccc(F)c(O)c2F)s1] |
| 12 | 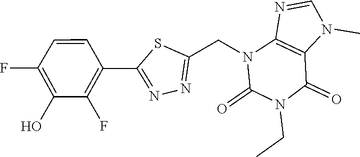 [SMILES: CCn1c(=O)c2c(ncn2C)n(Cc2nnc(-c3ccc(F)c(O)c3F)s2)c1=O] |
| 13 | 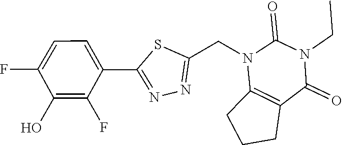 [SMILES: CCn1c(=O)c2c(n(Cc3nnc(-c4ccc(F)c(O)c4F)s3)c1=O)CCC2] |
| 14 | 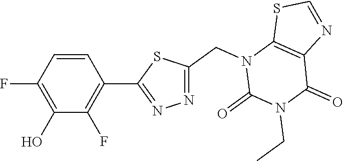 [SMILES: CCn1c(=O)c2ncsc2n(Cc2nnc(-c3ccc(F)c(O)c3F)s2)c1=O] |

| | | Reaction conditions (deviation from |
|---|---|---|
| Ex. | Starting materials | general procedure) |
| 2 | Intermediate I.1 | used catalyst: PdC1₂(dtbpf) |
| | and | used base: Na₂CO₃ |
| | 2,4,6-trifluoro-3-hydroxy | 3 h at 80° C.; purification by reversed |
| | phenylboronic acid | phase (SunFire C18; water/ACN/TFA) |
| 3 | Intermediates I.4 | used catalyst: XPhos Pd G2 |
| | and | used base: Na₂CO₃ |
| | III.1 | 10 min at 120° C. (microwave); |
| | | purification by reversed |
| | | phase (SunFire C18; water/ACN/TFA) |
| 4 | Intermediates I.4 + 1 | used catalyst: XPhos Pd G2 |
| | and | used base: Na₂CO₃ |
| | III.1 | 2 h at 85° C.; purification |
| | | by reversed phase (SunFire |
| | | C18; water/ACN/TFA) |
| 5 | Intermediates XII.2 | purification by forming precipitate |
| | and | with ACN |
| | III.1 | |
| 6 | Intermediates XII.3 | stirred for 1.75 h at 80° C.; |
| | and | purification by reversed phase |
| | III.1 | (SunFire C18; water/ACN/TFA) |
| 7 | Intermediates XII.4 | stirred for 1 h at 90° C.; |
| | and | purification by extraction from |
| | III.1 | water with DCM |
| 8 | Intermediates XIV.1 | stirred for 1.25 h at 80° C.; |
| | and | purification by reversed phase |
| | III.1 | (SunFire C18; water/ACN/TFA) |
| 9 | Intermediates XII.5 | stirred for 1.5 h at 80° C.; |
| | and | purification by reversed phase |
| | III.1 | (SunFire C18; water/ACN/TFA) |
| 10 | Intermediates XII.6 | stirred for 1.5 h at 80° C.; |
| | and | purification by reversed phase |
| | III.1 | (SunFire C18; water/ACN/TFA) |
| 11 | Intermediates XVIII.1 | stirred for 1.25 h at 80° C.; |
| | and | purification by reversed phase |
| | III.1 | (SunFire C18; water/ACN/TFA) |
| 12 | Intermediates XX.1 | stirred for 2 h at 80° C.; |
| | and | purification by reversed phase |
| | III.1 | (SunFire C18; water/ACN/TFA) |
| 13 | Intermediates XII.7 | stirred for 1 h at 90° C.; |
| | and | purification by performing |
| | III.1 | precipitate from ACN |
| 14 | Intermediates XX.2 | stirred for 2 h at 80° C.; |
| | and | purification by reversed phase |
| | III.1 | (SunFire C18; water/ACN/TFA) |

Analytical data for the example compounds 2-14 described in the tables above

| | | HPLC | |
|---|---|---|---|
| | | Retention | |
| | ESI- | time | |
| Ex. | MS | (method) | ¹H NMR |
| 2 | 370 | 0.51 | ¹H NMR (400 MHz, DMSO-d₆) δ |
| | | (C) | ppm: 3.17 (s, 3 H), 5.22 (s, 2 H), 5.78 |
| | | | (d, J = 7.86 Hz, 1 H), 7.37 (td, |
| | | | J = 10.84, 2.15 Hz, 1 H), 7.91 (d, J = 7.86 |
| | | | Hz, 1 H), 8.05 (s, 1 H), 10.45 (s, 1 H). |
| 3 | 353 | 0.55 min | ¹H NMR (400 MHz, DMSO-d₆) δ |
| | | (D) | ppm: 3.17 (s, 3 H), 5.47 (s, 2 H), 5.82 |
| | | | (d, J = 7.86 Hz, 1 H), 7.19-7.32 |
| | | | (m, 1 H), 7.58-7.70 (m, 1 H), |
| | | | 7.91 (d, J = 7.98 Hz, 1 |
| | | | H), 10.76 (s, 1 H). |
| 4 | 352 | 0.67 min | ¹H NMR (400 MHz, DMSO-d₆) δ |
| | | (D) | ppm: 3.17 (s, 3 H), 5.13 (s, 2 H), 5.78 |
| | | | (d, J = 7.86 Hz, 1 H), 7.38-7.53 |
| | | | (m, 2 H), 7.70 (s, 1 H), 7.82 |
| | | | (d, J = 7.86 Hz, 1 H), |
| | | | 10.21 (s, 1 H). |
| 5 | 365 | 0.84 min | ¹H NMR (400 MHz, DMSO-d₆) |
| | (M − H)⁻ | (A) | δ ppm: 1.09 (t, J = 7.03 Hz, 3 H), 3.84 |
| | | | (q, J = 7.05 Hz, 2 H), 5.46 (s, 2 |
| | | | H), 5.80 (d, J = 7.86 Hz, 1 H), |
| | | | 7.18-7.32 (m, 1 H), 7.64 (ddd, J = 8.87, |
| | | | 7.54, 5.89 Hz, 1 H), 7.89 (d, |
| | | | J = 7.98 Hz, 1 H), 10.76 (br s, 1 H). |
| 6 | 380 | 0.93 min | ¹H NMR (400 MHz, DMSO-d₆) δ ppm: |
| | | (A) | 1.09 (t, J = 7.03 Hz, 3 H), 1.81 |
| | | | (s, 3 H), 3.86 (q, J = 7.01 Hz, 2 |
| | | | H), 5.16 (s, 2 H), 7.13-7.24 (m, 1 H), |
| | | | 7.59 (td, J = 8.36, 6.08 Hz, 1 H), |
| | | | 7.77 (d, J = 0.89 Hz, 1 H), |
| | | | 7.99 (d, J = 2.03 Hz, 1 H), 10.59 (br s, 1 H). |
| 7 | 381 | 0.90 min | ¹H NMR (400 MHz, DMSO-d₆) δ ppm: |
| | | (A) | 1.10 (t, J = 7.03 Hz, 3 H), 1.84 (d, J = 1.01 |
| | | | Hz, 3 H), 3.87 (q, J = 6.97 |
| | | | Hz, 2 H), 5.42 (s, 2 H), 7.25 |
| | | | (td, J = 9.57, 1.77 Hz, 1 H), 7.64 (ddd, |
| | | | J = 8.93, 7.54, 5.83 Hz, 1 H), 7.79 (q, |
| | | | J = 1.14 Hz, 1 H), 10.75 (s, 1 H). |
| 8 | 381 | 0.89 min | ¹H NMR (400 MHz, DMSO-d₆) δ ppm: |
| | | (A) | 1.09 (t, J = 7.03 Hz, 3 H), 2.37 |
| | | | (s, 3 H), 3.84 (q, J = 6.97 Hz, 2 |
| | | | H), 5.51 (s, 2 H), 5.73 (s, 1 H), 7.20-7.32 |
| | | | (m, 1 H), 7.65 (ddd, J = 8.93, 7.54, |
| | | | 5.83 Hz, 1 H), 10.76 (br s, 1 H). |
| 9 | 420 | 0.95 min | ¹H NMR (400 MHz, DMSO-d₆) δ |
| | | (A) | ppm: 4.64 (q, J = 9.12 Hz, 2 H), 5.24 |
| | | | (s, 2 H), 5.87 (d, J = 7.98 Hz, 1 |
| | | | H), 7.12-7.24 (m, 1 H), 7.60 (ddd, |
| | | | J = 8.90, 7.89, 6.02 Hz, 1 H), 7.93-8.09 |
| | | | (m, 2 H), 10.59 (br s, 1 H). |
| 10 | 392 | 0.95 min | ¹H NMR (400 MHz, DMSO-d₆) δ |
| | | (A) | ppm: 0.27-0.49 (m, 4 H), 1.06-1.20 |
| | | | (m, 1 H), 3.70 (d, J = 7.10 Hz, 2 H), |
| | | | 5.21 (s, 2 H), 5.77 (d, J = 7.86 Hz, |
| | | | 1 H), 7.11-7.24 (m, 1 H), 7.60 (td, |
| | | | J = 8.40, 6.02 Hz, 1 H), 7.90 (d, |
| | | | J = 7.86 Hz, 1 H), 8.00 (d, J = 2.15 |
| | | | Hz, 1 H), 10.59 (s, 1 H). |
| 11 | 359 | 0.97 min | ¹H NMR (400 MHz, DMSO-d₆) δ |
| | | (A) | ppm: 3.64 (s, 2 H), 5.18 (s, 2 H), 7.02 |
| | | | (td, J = 7.41, 1.01 Hz, 1 H), 7.11- |
| | | | 7.22 (m, 2 H), 7.21-7.32 (m, 2 H), |
| | | | 7.57 (ddd, J = 8.93, 7.92, 5.96 Hz, |
| | | | 1 H), 8.05 (d, J = 2.28 Hz, 1 |
| | | | H), 10.56 (s, 1 H). |
| 12 | 421 | 0.78 min | ¹H NMR (400 MHz, DMSO-d₆) δ |
| | | (A) | ppm: 1.14 (br t, J = 6.91 Hz, 3 H), |
| | | | 3.84-4.03 (m, 5 H), 5.65 (s, 2 H), |
| | | | 7.23 (br t, J = 9.38 Hz, 1 H), |
| | | | 7.55-7.68 (m, 1 H), 8.06 |
| | | | (s, 1 H). |
| 13 | 407 | 0.86 min | ¹H NMR (400 MHz, DMSO-d₆) δ |
| | | (A) | ppm: 1.09 (t, J = 6.97 Hz, 3 H), 2.01 |
| | | | (quin, J = 7.54 Hz, 2 H), 2.60 |
| | | | (br t, J = 7.35 Hz, 2 H), 3.03 |
| | | | (br t, J = 7.60 Hz, 2 H), |
| | | | 3.85 (q, J = 6.97 Hz, 2 H), |
| | | | 5.44 (s, 2 H), 7.09-7.42 |
| | | | (m, 1 H), 7.64 (ddd, J = 8.90, |
| | | | 7.51, 5.89 Hz, 1 H), 10.76 (br s, 1 H). |
| 14 | 424 | 0.78 min | ¹H NMR (400 MHz, DMSO-d₆) δ |
| | | (A) | ppm: 1.17 (t, J = 6.97 Hz, 3 H), 3.97 (q, |
| | | | J = 6.97 Hz, 2 H), 5.68 (s, 2 H), |
| | | | 7.20-7.33 (m, 1 H), 7.64 (ddd, J = 8.84, 7.51, |
| | | | 5.83 Hz, 1 H), 8.83 (s, 1 H), 10.77 (s, 1 H). |

### 3.3 Example Compound 15

1-{[5-(2,4-Difluoro-3-hydroxyphenyl)-1,3-thiazol-2-yl]methyl}-3-methyl-1,2,3,4-tetrahydropyrimidine-2,4-dione

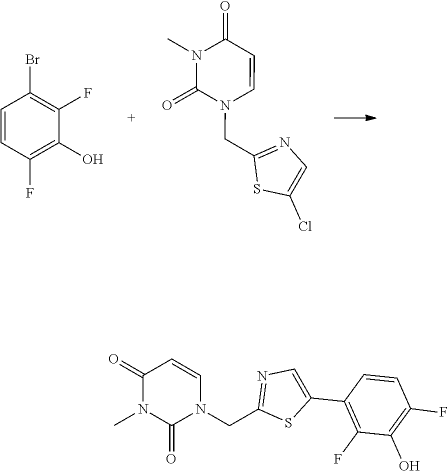 [SMILES: Oc1c(F)ccc(Br)c1F ; Cn1c(=O)ccn(Cc2ncc(Cl)s2)c1=O ; Cn1c(=O)ccn(Cc2ncc(-c3ccc(F)c(O)c3F)s2)c1=O]

3-Bromo-2,6-difluorophenol (0.04 g; 0.18 mmol), bis(pinacolato)diboron (0.05 g; 0.18 mmol), XPhos Pd G2 (0.01 g; 0.01 mmol), XPhos (0.01 g; 0.01 mmol) and KOAc (0.04 g; 0.36 mmol) in EtOH (2 mL) were stirred for 10 min at 120° C. in a microwave. Intermediate 1.2 (0.03 g; 0.12 mmol), Na₂CO₃ (aq. solution; 2 mol/L; 0.18 mL; 0.36 mmol) and XPhos Pd G2 (0.01 g; 0.01 mmol) were added and stirred for another 10 min at 120° C. in the microwave. The reaction mixture was diluted with DMF and purified by column chromatography (reversed phase; Sunfire C18; water/ACN/TFA) to provide the desired product.
C₁₅H₁₁F₂N₃O₃S (M=351.3 g/mol)
ESI-MS: 352 [M+H]⁺
Rₜ (HPLC): 0.61 min (method D)
Yield: 0.01 g; 31%
¹H NMR (400 MHZ, DMSO-d₆) δ ppm: 3.17 (s, 3H), 5.29 (s, 2H), 5.80 (d, J=7.86 Hz, 1 H), 7.09-7.25 (m, 2H), 7.87 (d, J-7.86 Hz, 1H), 8.10 (s, 1H), 10.49 (s, 1H).

### 3.4 Example Compound 16

1-{[3-(2,4-Difluoro-3-hydroxyphenyl)-1,2-oxazol-5-yl]methyl}-3-methyl-1,2,3,4-tetrahydropyrimidine-2,4-dione

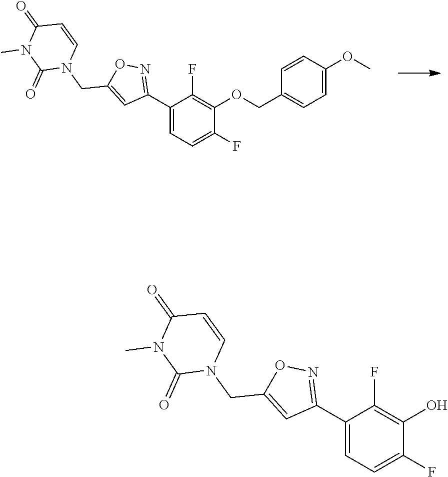

Intermediate VI.1 (0.40 g; 1.32 mmol) in TFA (1.00 mL; 12.96 mmol) and DCM (3 mL) was stirred at RT for 20 min. The reaction mixture was concentrated under reduced pressure. The residue was purified by column chromatography (reversed phase; Sunfire C18; water/ACN/TFA) to provide the desired product.
C₁₅H₁₁F₂N₃O₄ (M=335.3 g/mol)
ESI-MS: 336 [M+H]⁺
Rₜ (HPLC): 0.83 min (method A)
Yield: 0.01 g; 72%
¹H NMR (400 MHZ, DMSO-d₆) δ ppm: 3.17 (s, 3H), 5.20 (s, 2H), 5.82 (d, J=7.86 Hz, 1 H), 6.89 (d, J=2.66 Hz, 1H), 7.10-7.22 (m, 1H), 7.28 (ddd, J=8.71, 7.57, 5.89 Hz, 1H), 7.86 (d, J=7.86 Hz, 1H), 10.57 (s, 1H).

The following example compounds 17 to 22 were prepared using procedures analogous to those described under 3.4 above using appropriate starting materials. As is appreciated by those skilled in the art, these analogous examples may involve variations in general reaction conditions.

| | | | Reaction conditions |
|---|---|---|---|
| | Starting | | (deviation from |
| Ex. | materials | Structure | general procedure) |
| 17 | Intermediate VI.2 | 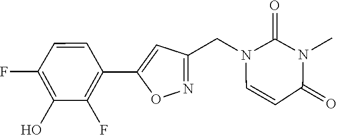 [SMILES: Cn1c(=O)ccn(Cc2cc(-c3ccc(F)c(O)c3F)on2)c1=O] | |
| 18 | Intermediate XII.1 | 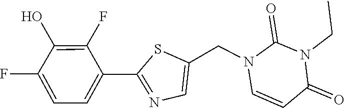 [SMILES: CCn1c(=O)ccn(Cc2cnc(-c3ccc(F)c(O)c3F)s2)c1=O] | |
| 19 | Intermediate XVI.1 | 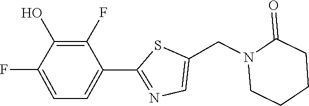 [SMILES: O=C1CCCCN1Cc1cnc(-c2ccc(F)c(O)c2F)s1] | |
| 20 | Intermediate XVI.2 | 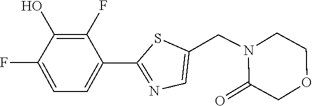 [SMILES: O=C1COCCN1Cc1cnc(-c2ccc(F)c(O)c2F)s1] | |
| 21 | Intermediate XVI.3 | 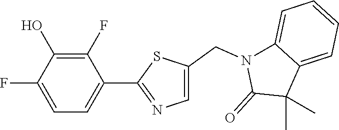 [SMILES: CC1(C)C(=O)N(Cc2cnc(-c3ccc(F)c(O)c3F)s2)c2ccccc21] | |
| 22 | Intermediate XVI.4 | 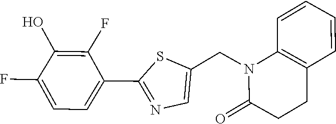 [SMILES: O=C1CCc2ccccc2N1Cc1cnc(-c2ccc(F)c(O)c2F)s1] | |

Analytical data for the compounds described in the table above:

| | | HPLC | |
|---|---|---|---|
| | | retention | |
| | ESI- | time | |
| Ex. | MS | (method) | ¹H NMR |
| 17 | 336 | 0.82 min | ¹H NMR (400 MHz, DMSO-d₆) δ ppm: |
| | | (A) | 3.17 (s, 3 H), 5.10 (s, 2 H), 5.78 (d, |
| | | | J = 7.86 Hz, 1 H), 6.79 (d, J = 3.30 Hz, 1 |
| | | | H), 7.07-7.14 (m, 2 H), 7.83 (d, J = 7.86 |
| | | | Hz, 1 H). |
| 18 | 366 | 0.88 min | ¹H NMR (400 MHz, DMSO-d₆) δ ppm: |
| | | (A) | 1.09 (t, J = 7.03 Hz, 3 H), 3.84 (q, |
| | | | J = 7.05 Hz, 2 H), 5.20 (s, 2 H), 5.75 (d, |
| | | | J = 7.98 Hz, 1 H), 7.10-7.29 (m, 1 H), |
| | | | 7.59 (td, J = 8.33, 6.02 Hz, 1 H), 7.88 (d, |
| | | | J = 7.98 Hz, 1 H), 7.99 (d, J = 2.28 Hz, 1 |
| | | | H), 10.60 (br s, 1 H). |
| 19 | 325 | 0.86 min | ¹H NMR (400 MHz, DMSO-d₆) δ ppm: |
| | | | 1.59-1.79 (m, 4 H), 2.27 (t, J = 6.27 Hz, |
| | | (A) | 2 H), 3.29 (t, J = 5.70 Hz, 2 H), 4.69 (s, 2 |
| | | | H), 7.18 (td, J = 9.54, 1.84 Hz, 1 H), 7.60 |
| | | | (ddd, J = 8.90, 7.95, 5.96 Hz, 1 H), 7.87 |
| | | | (d, J = 2.28 Hz, 1 H), 10.56 (br s, 1 H). |
| 20 | 327 | 0.79 min | ¹H NMR (400 MHz, DMSO-d₆) δ ppm: |
| | | (A) | 3.34-3.44 (m, 2 H), 3.77-3.88 (m, 2 H), |
| | | | 4.09 (s, 2 H), 4.77 (s, 2 H), 7.19 (td, |
| | | | J = 9.57, 1.77 Hz, 1 H), 7.61 (ddd, |
| | | | J = 8.93, 7.92, 5.96 Hz, 1 H), 7.91 (d, |
| | | | J = 2.41 Hz, 1 H), 10.57 (s, 1 H). |
| 21 | 387 | 1.04 min | ¹H NMR (400 MHz, DMSO-d₆) δ ppm: |
| | | (A) | 1.29 (s, 6 H), 5.20 (s, 2 H), 7.02-7.09 |
| | | | (m, 1 H), 7.12-7.28 (m, 3 H), 7.36 (d, |
| | | | J = 7.22 Hz, 1 H), 7.56 (ddd, J = 8.93, |
| | | | 7.92, 5.96 Hz, 1 H), 8.04 (d, J = 2.28 Hz, |
| | | | 1 H), 10.55 (br s, 1 H). |
| 22 | 373 | 0.99 min | ¹H NMR (400 MHz, DMSO-d₆) δ ppm: |
| | | (A) | 2.65 (dd, J = 8.62, 6.21 Hz, 2 H), 2.83- |
| | | | 2.94 (m, 2 H), 5.37 (s, 2 H), 7.00 (td, |
| | | | J = 7.35, 1.01 Hz, 1 H), 7.13-7.19 (m, 1 |
| | | | H), 7.21-7.27 (m, 2 H), 7.27-7.33 (m, 1 |
| | | | H), 7.56 (ddd, J = 8.87, 7.98, 5.96 Hz, 1 |
| | | | H), 8.00 (d, J = 2.28 Hz, 1 H), 10.54 (br |
| | | | s, 1 H). |

### 3.5 Example Compound 23

1-{[3-(2,4-Difluoro-3-hydroxyphenyl)-1,2,4-oxadiazol-5-yl]methyl}-3-methyl-1,2,3,4-tetrahydropyrimidine-2,4-dione

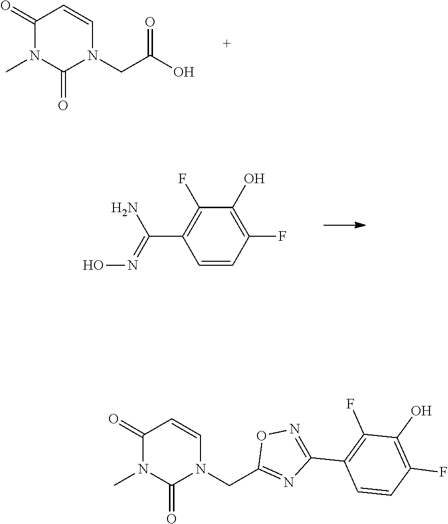 [SMILES: Cn1c(=O)ccn(CC(=O)O)c1=O ; N/C(=N\O)c1ccc(F)c(O)c1F ; Cn1c(=O)ccn(Cc2nc(-c3ccc(F)c(O)c3F)no2)c1=O]

A mixture of 2-(3-methyl-2,4-dioxo-1,2,3,4-tetrahydropyrimidin-1-yl) acetic acid (0.04 g; 0.20 mmol), EDC hydrochloride (0.04 g; 0.20 mmol) and HOBT (0.03 g; 0.24 mmol) in DMF (1 mL) was stirred at RT for 15 min. This mixture was added to Intermediate X.1 (0.04 g; 0.20 mmol) in DMF (1 mL). The resulting reaction mixture was stirred for 2 h at 95° C. and overnight at RT. The reaction mixture was diluted with DMF/water, filtered and purified by column chromatography (reversed phase; Sunfire C18; water/ACN/TFA) to provide the desired product.
C₁₄H₁₀F₂N₂O₄ (M=336.3 g/mol)
ESI-MS: 337 [M+H]⁺
Rₜ (HPLC): 0.47 min (method C)
Yield: 0.01 g; 6%
¹H NMR (400 MHZ, DMSO-d₆) δ ppm: 3.17 (s, 3H), 5.40 (s, 2H), 5.87 (d, J=7.98 Hz, 1 H), 7.22 (td, J=9.54, 1.71 Hz, 1H), 7.40 (ddd, J=8.87, 7.35, 5.96 Hz, 1H), 7.88 (d, J=7.98 Hz, 1H), 10.67 (s, 1H).

### 3.6 Example Compound 24

1-{[2-(2,4-difluoro-3-hydroxyphenyl)-1,3-thiazol-5-yl]methyl}-3-methyl-1,3-diazinan-2-one

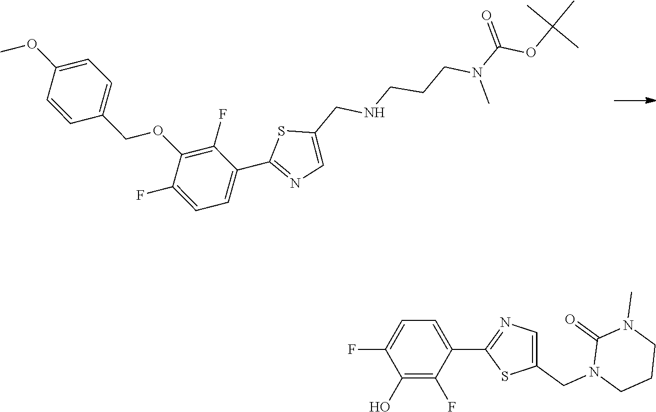 [SMILES: COc1ccc(COc2c(F)ccc(-c3ncc(CNCCCN(C)C(=O)OC(C)(C)C)s3)c2F)cc1 ; CN1CCCN(Cc2cnc(-c3ccc(F)c(O)c3F)s2)C1=O]

Intermediate XVII.1 (0.03 g; 0.06 mmol) and TFA (1.00 mL; 12.96 mmol) in DCM (3 mL) were stirred at RT for 25 min. The reaction mixture was concentrated under reduced pressure. The residue was dissolved in THF (3 mL) and CDI (0.01 g; 0.07 mmol) was added. After stirring at 50° C. for 2 h, the reaction mixture was diluted with DMF and water, and purified by column chromatography (reversed phase; Sunfire C18; water/ACN/TFA) to provide the desired product.
C₁₅H₁₅F₂N₃O₂S (M=339.4 g/mol)
ESI-MS: 340 [M+H]⁺
Rₜ (HPLC): 0.86 min (method A)
Yield: 0.01 g; 52%
¹H NMR (400 MHZ, DMSO-d₆) δ ppm: 1.86 (quin, J=5.92 Hz, 2H), 2.82 (s, 3H), 3.22 (dt, J=20.40, 5.89 Hz, 4H), 4.63 (s, 2H), 7.13-7.22 (m, 1H), 7.60 (ddd, J=8.78, 8.02, 6.02 Hz, 1H), 7.83 (d, J=2.28 Hz, 1H), 10.55 (br s, 1H).

### 3.7 Example Compound 25 (General Procedure)

1-{[5-(2,4-Difluoro-3-hydroxyphenyl)-1,3,4-thiadiazol-2-yl]methyl}-3-hydroxy-3-methyl-2,3-dihydro-1H-indol-2-one

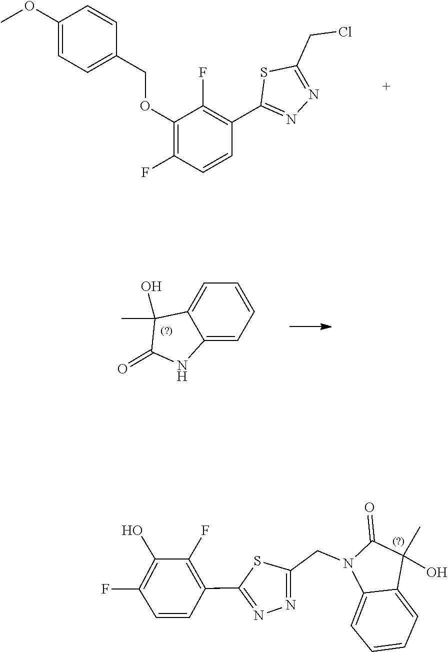 [SMILES: COc1ccc(COc2c(F)ccc(-c3nnc(CCl)s3)c2F)cc1 ; CC1(O)C(=O)Nc2ccccc21 ; CC1(O)C(=O)N(Cc2nnc(-c3ccc(F)c(O)c3F)s2)c2ccccc21]

Intermediate IX.3 (0.10 g; 0.26 mmol), 3-hydroxy-3-methyl-2,3-dihydro-1H-indol-2-one (0.04 g; 0.26 mmol) and K₂CO₃ (0.09 g; 0.65 mmol) in DMF (3 mL) were stirred for 20 h at 70° C. Water was added and the reaction mixture was extracted several times with DCM. The organic layer was separated, dried and concentrated under reduced pressure. The residue was purified by column chromatography (reversed phase; Sunfire C18; water/ACN/TFA) to give the intermediate. This intermediate was taken up in DCM (1.5 mL) and TFA (1.00 mL; 49.60 mmol) and stirred overnight at RT. After concentrating under reduced pressure, the residue was taken up in ACN and purified by column chromatography (reversed phase; Sunfire C18; water/ACN/TFA) to provide the desired product.
C₁₈H₁₃F₂N₃O₃S (M=389.4 g/mol)
ESI-MS: 390 [M+H]⁺
Rₜ (HPLC): 0.79 min (method A)
Yield: 0.01 g; 6%
¹H NMR (400 MHZ, DMSO-d₆) δ ppm: 1.44 (s, 3H), 5.42 (s, 2H), 6.09 (br s, 1H), 7.09 (td, J=7.45, 0.82 Hz, 1H), 7.13 (d, J=7.86 Hz, 1H), 7.24 (td, J=9.60, 1.84 Hz, 1H), 7.27-7.35 (m, 1H), 7.39 (dd, J=7.35, 0.76 Hz, 1H), 7.63 (ddd, J=8.93, 7.54, 5.83 Hz, 1 H), 10.74 (s, 1H).

The following example compounds 26 to 30 were prepared using procedures analogous to those described under 3.7 above using appropriate starting materials. As is appreciated by those skilled in the art, these analogous examples may involve variations in general reaction conditions.

| | | | Reaction conditions |
|---|---|---|---|
| | Starting | | (deviation from |
| Ex. | materials | Structure | general procedure) |
| 26 | Intermediate IX.3 and 6-bromo-3- hydroxy-3- methyl-2,3- dihydro-1H- indol-2- one | 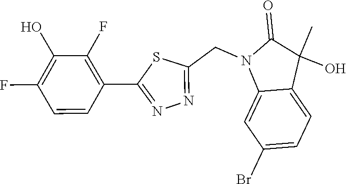 [SMILES: CC1(O)C(=O)N(Cc2nnc(-c3ccc(F)c(O)c3F)s2)c2cc(Br)ccc21] | |

Analytical data for example compound 26 described above

| | | HPLC | |
|---|---|---|---|
| | | retention | |
| | ESI- | time | |
| Ex. | MS | (method) | ¹H NMR |
| 26 | 468 | 0.88 min | ¹H NMR (400 MHz, DMSO-d₆) δ |
| | | (A) | ppm: 1.43 (s, 3 H), 5.45 (s, 2 H), 6.17 |
| | | | (br s, 1 H), 7.21-7.30 (m, 2 H), |
| | | | 7.31-7.36 (m, 1 H), 7.44 (d, J = 1.65 |
| | | | Hz, 1 H), 7.64 (ddd, J = 8.97, 7.57, |
| | | | 5.89 Hz, 1 H), 10.75 (s, 1 H). |

### 3.7 Example Compound 27

1-{[2-(2,4-Difluoro-3-hydroxyphenyl)-1,3-thiazol-5-yl]methyl}-2,3-dihydro-1H-1,3-benzodiazol-2-one

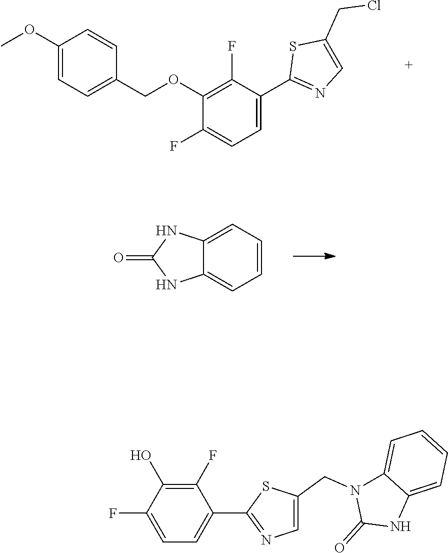 [SMILES: COc1ccc(COc2c(F)ccc(-c3ncc(CCl)s3)c2F)cc1 ; O=c1[nH]c2ccccc2[nH]1 ; O=c1[nH]c2ccccc2n1Cc1cnc(-c2ccc(F)c(O)c2F)s1]

2,3-Dihydro-1H-1,3-benzodiazol-2-one (0.03 g; 0.08 mmol) and sodium hydride (55% dispersion in mineral oil; 0.01 g; 0.14 mmol) in THF (1 mL) were stirred at RT for 15 min. Intermediate IX.2 (0.02 g; 0.11 mmol) was added. After stirring for 6 h at 60° C., the mixture was evaporated and taken up in water and DMF to be purified by column chromatography (reversed phase; XBridge C18; water/ACN/NH₄OH). The residue was taken up in TFA (solution in DCM; 50%; 1 mL) and stirred overnight at RT. The reaction mixture was concentrated under reduced pressure, the residue was taken up in DMF and purified by column chromatography (reversed phase; SunFire C18; water/ACN/TFA).
C₁₇H₁₁F₂N₃O₂S (M=359.4 g/mol)
ESI-MS: 360 [M+H]⁺
Rₜ (HPLC): 0.71 min (method E)
Yield: 0.01 g; 10%
¹H NMR (400 MHZ, DMSO-d₆) δ ppm: 5.30 (s, 2H), 6.97-7.05 (m, 3H), 7.12-7.21 (m, 1 H), 7.26-7.31 (m, 1H), 7.53-7.61 (m, 1H), 8.05 (d, J=2.28 Hz, 1H), 10.56 (s, 1H), 10.96 (s, 1H).

The following compounds 28 to 29 were prepared using procedures analogous to those described under 3.5 above using appropriate starting materials. As is appreciated by those skilled in the art, these analogous examples may involve variations in general reaction conditions.

| | | | Reaction |
|---|---|---|---|
| | | | conditions |
| | | | (deviation from |
| | Starting | | general |
| Ex. | materials | Structure | procedure) |
| 28 | Intermediate IX.2 and 1-methyl-2,3- dihydro-1H-1,3- benzodiazol-2- one | 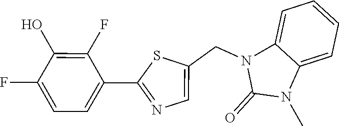 [SMILES: Cn1c(=O)n(Cc2cnc(-c3ccc(F)c(O)c3F)s2)c2ccccc21] | |
| 29 | Intermediate IX.2 and 2,3-dihydro-1,3- benzoxazol-2- one | 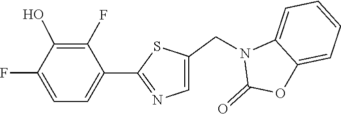 [SMILES: O=c1oc2ccccc2n1Cc1cnc(-c2ccc(F)c(O)c2F)s1] | instead of NaH 2.3 equivalents Cs₂CO₃ are used |

Analytical data for the compounds 28 and 29 described above

| | | HPLC | |
|---|---|---|---|
| | | retention | |
| | ESI- | time | |
| Ex. | MS | (method) | ¹H NMR |
| 28 | 374 | 0.78 min | ¹H NMR (400 MHz, DMSO-d₆) δ ppm: 3.35 |
| | | (E) | (s, 3 H), 5.36 (s, 2 H), 7.05-7.11 (m, 2 H), |
| | | | 7.12-7.21 (m, 2 H), 7.32-7.41 (m, 1 H), 7.56 |
| | | | (ddd, J = 8.90, 7.95, 5.96 Hz, 1 H), 8.06 (d, |
| | | | J = 2.28 Hz, 1 H), 10.56 (s, 1 H). |
| 29 | 361 | 0.97 min | ¹H NMR (400 MHz, DMSO-d₆) δ ppm: 5.38 (s, |
| | | (E) | 2 H), 7.13-7.21 (m, 2 H), 7.25 (td, J = 7.76, |
| | | | 1.08 Hz, 1 H), 7.36 (dd, J = 7.98, 0.63 Hz, 1 |
| | | | H), 7.47 (dd, J = 7.86, 0.76 Hz, 1 H), 7.54- |
| | | | 7.62 (m, 1 H), 8.12 (d, J = 2.28 Hz, 1 H), 10.60 |
| | | | (br s, 1 H). |

### 3.8 Example Compound 30

1-{[5-(2,4-Difluoro-3-hydroxyphenyl)-1,3,4-thiadiazol-2-yl]methyl}-3-ethyl-1,2,3,4-tetrahydroquinazoline-2,4-dione

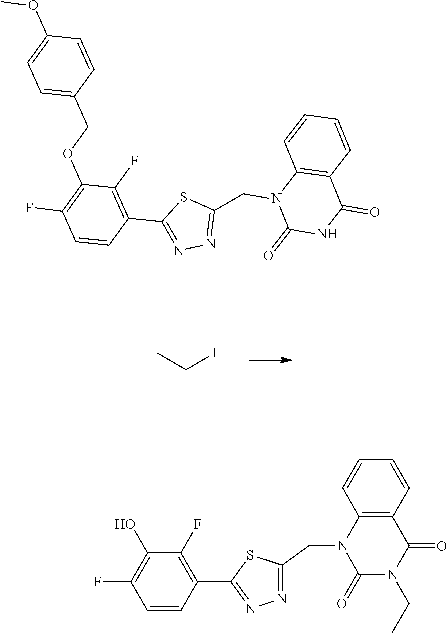

Intermediate XIII.4 (0.07 g; 0.14 mmol), iodoethane (0.02 mL; 0.21 mmol) and K₂CO₃ (0.04 g; 0.28 mmol) in DMF (1 mL) were stirred at 80° C. for 2 h. The reaction mixture was concentrated under reduced pressure and purified by column chromatography (reversed phase; Sunfire C18; water/ACN/TFA). The residue was taken up in TFA (1.00 mL; 12-96 mmol) and DCM (2.5 mL). After stirring overnight at RT the mixture was purified by column chromatography (reversed phase; Sunfire C18; water/ACN/TFA) to provide the desired compound.
C₁₉H₁₄F₂N₄O₃S (M=416.4 g/mol)
ESI-MS: 417 [M+H]⁺
Rₜ (HPLC): 0.90 min (method A)
Yield: 0.01 g; 16%
¹H NMR (400 MHZ, DMSO-d₆) δ ppm: 1.20 (t, J=7.03 Hz, 3H), 4.04 (q, J=7.05 Hz, 2 H), 5.84 (s, 2H), 7.24 (ddd, J=10.30, 8.90, 1.84 Hz, 1H), 7.31-7.37 (m, 1H), 7.59-7.64 (m, 1H), 7.65 (d, J=8.24 Hz, 1H), 7.76-7.81 (m, 1H), 8.10 (dd, J=7.86, 1.52 Hz, 1H), 10.75 (br s, 1H).

### Example 4:: Biochemical humanHSD17B13-RapidFire MS/MS Assay

Estradiol (Sigma, Cat #E8875), NAD (Roche, Cat #10621650001) and recombinant humanHSD17B13 (full-length HSD17B13 (Uniprot ID Q7Z5P4-1) with C-terminal His-tag, expressed in mammalian cells and purified to homogeneity) were diluted in assay buffer (100 mM Tris, Sigma, Cat #T2319; sodium chloride, Roth, Cat #3957.2; 0.5 mM EDTA, Invitrogen, Cat #15575020; 0.1% TCEP, Invitrogen, Cat #T2556; 0.05% BSA fraction V (protease and fatty acid free), Serva, Cat #11945; 0,001% Tween20, Serva, Cat #37470). Compounds were serially diluted in DMSO (Sigma, Cat #5879) and spotted on a 384-well Microplate, PP, V-bottom (Greiner, Cat #781280) plate by a Labcyte Echo 55x (1% DMSO in the Assay). First, 6 μL/well of recombinant hHSD17B13 (1 nM final) dilution was added, followed by 15 min incubation at RT. Second, 6 μL/well of diluted Estradiol (30 μM final) and NAD (0.5 mM final) was added, mixed and incubated for 4 h at RT. 1 μL d4-Estrone (50 nM final; Sigma, Cat #489204) followed by 2.4 μL Girard's Reagent P (6.5 mM final; TCl, Cat #G0030) dissolved in 90% (Sigma, Cat #34860) methanol and 10% formic acid (Merck, Cat #33015) was added to derivatize analytes and stop the enzyme reaction. Incubation was for 12-24 h at RT before adding 70 μL dH2O.

The analytical sample handling was performed by a rapid-injecting RapidFire autosampler system (Agilent, Waldbronn, Germany) coupled to a triple quadrupole mass spectrometer (Triple Quad 6500, AB Sciex Germany GmbH, Darmstadt, Germany). Liquid sample was aspirated by a vacuum pump into a 10 μL. sample loop for 250 ms and subsequently flushed for 3000 ms onto a C18 cartridge (Agilent, Waldbronn, Germany) with the aqueous mobile phase (99.5% water, 0.49% acetic acid, 0.01% Trifluoroacetic acid, flow rate 1.5 mL/min). The solid phase extraction step retained the analyte while removing interfering matrix (e.g., buffer components). The analyte was desorbed and eluted back from the cartridge for 3000 ms with an organic mobile phase (49.75% methanol, 49,75% acetonitrile, 0.49% acetic acid, 0.01% trifluoroacetic acid, flow rate 1.25 mL/min) and flushed into the mass spectrometer for detection in MRM mode. The MRM transition for the Estrone was 404.1 <157.1 Da (declustering potential 27V, collision energy 43 V) and for the internal standard D4 Estrone was 408.1<159.1 Da (declustering potential 27V, collisionenergy 43 V). Dwell time for each MRM transition was 25 ms and pause time between MRMs was 5 ms. The mass spectrometer was operated in positive ionization mode (curtain gas 35 Au, collision gas medium, ion spray voltage 4200 V, temperature 550° C., ion source gas 1 65 Au, ion source gas 2 80 Au). While performing the back flush into the mass spectrometer, the sample loop and relevant tubing were flushed with the organic mobile phase to prevent carryover of analyte or matrix components into the next sample. Equilibration time for the system was 500 ms. To minimize carryover effects, the wash station of the RapidFire system was used to perform needle washes with pure water (100%) and pure methanol (100%) between samples. The solvent delivery setup of the RapidFire system consists of two continuously running and isocratically operating HPLC pumps (G1310A, Agilent, Waldbronn, Germany) and one binary HPLC pump channel B (G4220A, Agilent, Waldbronn, Germany). MS data processing was performed in GMSU (Alpharetta, GA, USA), and peak area ratio analyte/internal standard was reported for IC₅₀ calculation. IC₅₀ values were calculated using a 4-parameter non-linear regression curve fitting model (Software Megalab inhouse development). For data evaluation and calculation, the measurement of the bottom (no HSD17B13 enzyme) was set as 0% control and the measurement of the top (includes NAD, Estrone and HSD17B13) was set as 100% control. The IC₅₀ values were calculated using the standard 4 parameter logistic regression formula:

$$Y = Bottom + ( Top - Bottom ) / ( 1 + 10 \hat{} ( ( Log IC 50 - X ) \times Slope + log ( ( Top - Bottom ) / ( Fifty - Bottom ) - 1 ) ) ) .$$

### Example 5: Biochemical mouseHSD17B13-RapidFire MS/MS Assay

Estradiol (Sigma, Cat #E8875), NAD (Roche, Cat #10621650001) and recombinant mouseHSD17B13 (U-Protein Express BV, Netherlands) were diluted in assay buffer (100 mM Tris, Sigma, Cat #T2319; sodium chloride, Roth, Cat #3957.2; 0.5 mM EDTA, Invitrogen, Cat #15575020; 0.1% TCEP, Invitrogen, Cat #T2556; 0.05% BSA fraction V (protease and fatty acid free), Serva, Cat #11945; 0,001% Tween20, Serva, Cat #37470). Compounds were serially diluted in DMSO (Sigma, Cat #5879) and spotted on a 384-well Microplate, PP, V-bottom (Greiner, Cat #781280) plate by a Labcyte Echo 55x (1% DMSO in the Assay). First, 6 μL/well of recombinant mouseHSD17B13 (50 nM final) dilution was added, followed by 15 min incubation at RT. Second, 6 μL/well of diluted Estradiol (30 μM final) and NAD (0.5 mM final) was added, mixed and incubated for 3 h at RT. Adding 1 μL d4-Estrone (50 nM final; Sigma, Cat #489204) followed by 2.4 μL Girard's Reagent P (6.5 mM final; TCl, Cat #G0030) dissolved in 90% (Sigma, Cat #34860) Methanol and 10% formic acid (Merck, Cat #33015) to derivatize analytes and stop the enzyme reaction. Incubation was for 12-24 h at RT before adding 70 μL dH2O.

The analytical sample handling was performed by a rapid-injecting RapidFire autosampler system (Agilent, Waldbronn, Germany) coupled to a triple quadrupole mass spectrometer (Triple Quad 6500, AB Sciex Germany GmbH, Darmstadt, Germany). Liquid sample was aspirated by a vacuum pump into a 10 μL sample loop for 250 ms and subsequently flushed for 3000 ms onto a C18 cartridge (Agilent, Waldbronn, Germany) with the aqueous mobile phase (99.5% water, 0.49% acetic acid, 0.01% trifluoroacetic acid, flow rate 1.5 mL/min). The solid phase extraction step retained the analyte while removing interfering matrix (e.g., buffer components). The analyte was desorbed and eluted back from the cartridge for 3000 ms with an organic mobile phase (49.75% methanol, 49,75% acetonitrile, 0.49% acetic acid, 0.01% trifluoroacetic acid, flow rate 1.25 mL/min) and flushed into the mass spectrometer for detection in MRM mode. The MRM transition for the Estrone was 404.1<157.1 Da (declustering potential 27V, collision energy 43 V) and for the internal standard D4 Estrone was 408.1<159.1 Da (declustering potential 27V, collisionenergy 43 V). Dwell time for each MRM transition was 25 ms and pause time between MRMs was 5 ms. The mass spectrometer was operated in positive ionization mode (curtain gas 35 Au, collision gas medium, ion spray voltage 4200 V, temperature 550° C., ion source gas 1 65 Au, ion source gas 2 80 Au). While performing the back flush into the mass spectrometer, the sample loop and relevant tubing were flushed with the organic mobile phase to prevent carryover of analyte or matrix components into the next sample. Equilibration time for the system was 500 ms. To minimize carryover effects, the wash station of the RapidFire system was used to perform needle washes with pure water (100%) and pure methanol (100%) between samples. The solvent delivery setup of the RapidFire system consisted of two continuously running and isocratically operating HPLC pumps (G1310A, Agilent, Waldbronn, Germany) and one binary HPLC pump channel B (G4220A, Agilent, Waldbronn, Germany). MS data processing was performed in GMSU (Alpharetta, GA, USA), and peak area ratio analyte/internal standard was reported for IC₅₀ calculation. IC₅₀ values were calculated using a 4-parameter non-linear regression curve fitting model (Software Megalab inhouse development). For data evaluation and calculation, the measurement of the bottom (no HSD17B13 enzyme) was set as 0% control and the measurement of the top (includes NAD, Estrone and HSD17B13) was set as 100% control. The IC₅₀ values were calculated using the standard 4 parameter logistic regression formula: Y═Bottom+(Top−Bottom)/(1+10\hat{ }((LogIC50−X)×Slope+log ((Top−Bottom)/(Fifty−Bottom)−1))).

### Example 6: Cellular Human HSD17B13-RapidFire MS/MS Assay

Estradiol (Sigma, Cat #E8875) dilution and cells (clonal HEK293 cells stabily overexpressing humanHSD17B13-Myc/DDK tagged, Lakepharma) were prepared in serum free medium (DMEM, Sigma, Cat #D5796; 10% heat inactivated FBS, Gibco, Cat #100500; 1×Glutamax, Gibco, Cat #35050-087; 1×sodium pyruvate, Gibyo, Cat #11360070). 25 μL of a 0,4*10\hat{ }6 cells/mL dilution was seeded on a 384-well Microplate (culture-plate, Perkin Elmer, Cat #6007680) 24 h prior to compound testing.

Compounds were serially diluted in DMSO (Sigma, Cat #5879) and spotted on the pre-seeded cell plate, by a Labcyte Echo 55x (1% DMSO in the Assay) and incubated for 30 min at 37° C. in a humidified incubator (rH=95%, CO2=5%). Afterwards 25 μL of 60 μM Estradiol dilution was added to the plate and incubated for 3 h at 37° C. in a humidified incubator (rH=95%, CO2=5%).

20 μL supernatant were taken and 2.5 μL d4-Estrone (50 nM final; Sigma, Cat #489204) were added followed by 5 μL Girard's Reagent P (6.5 mM final; TCl, Cat #G0030) dissolved in 90% (Sigma, Cat #34860) methanol and 10% formic acid (Merck, Cat #33015) to derivatize analytes. Incubabation was for 12-24 h at RT before adding 70 μL dH2O.

The analytical sample handling was performed by a rapid-injecting RapidFire autosampler system (Agilent, Waldbronn, Germany) coupled to a triple quadrupole mass spectrometer (Triple Quad 6500, AB Sciex Germany GmbH, Darmstadt, Germany). Liquid sample was aspirated by a vacuum pump into a 10 μL. sample loop for 250 ms and subsequently flushed for 3000 ms onto a C18 cartridge (Agilent, Waldbronn, Germany) with the aqueous mobile phase (99.5% water, 0.49% acetic acid, 0.01% trifluoroacetic acid, flow rate 1.5 mL./min). The solid phase extraction step retained the analyte while removing interfering matrix (e.g., buffer components). The analyte was desorbed and eluted back from the cartridge for 3000 ms with an organic mobile phase (49.75% methanol, 49,75% acetonitrile, 0.49% acetic acid, 0.01% trifluoroacetic acid, flow rate 1.25 mL/min) and flushed into the mass spectrometer for detection in MRM mode. The MRM transition for the Estrone was 404.1<157.1 Da (declustering potential 27V, collision energy 43 V) and for the internal standard D4 Estrone was 408.1<159.1 Da (declustering potential 27V, collisionenergy 43 V). Dwell time for each MRM transition was 25 ms and pause time between MRMs is 5 ms. The mass spectrometer was operated in positive ionization mode (curtain gas 35 Au, collision gas medium, ion spray voltage 4200 V, temperature 550° C., ion source gas 1 65 Au, ion source gas 2 80 Au). While performing the back flush into the mass spectrometer, the sample loop and relevant tubing were flushed with the organic mobile phase to prevent carryover. MS data processing was performed in GMSU (Alpharetta, GA, USA), and peak area ratio analyte/internal standard was reported for IC₅₀ calculation. IC₅₀ values were calculated using a 4-parameter non-linear regression curve fitting model (Software Megalab inhouse development). For data evaluation and calculation, the measurement of the bottom (cells with Estradiol and 10 μM of an inhouse identified HSD17B13 inhibitor) was set as 0% control and the measurement of the top (includes cells with Estradiol) was set as 100% control. The IC₅₀ values were calculated using the standard 4 parameter logistic regression formula: Y═Bottom+(Top−Bottom)/(1+10\hat{ }((LogIC50−X)×Slope+log ((Top−Bottom)/(Fifty−Bottom)−1))).

### Example 7: Cellular Human HSD17B13 Viability Assay

Estradiol (Sigma, Cat #E8875) dilution and cells (clonal HEK293 cells stably overexpressing humanHSD17B13-Myc/DDK tagged, Lakepharma) were prepared in serum free medium (DMEM, Sigma, Cat #D5796; 10% heat inactivated FBS, Gibco, Cat #100500; 1×Glutamax, Gibco, Cat #35050-087; 1×sodium pyruvate, Gibyo, Cat #11360070). 25 μL of a 0,4*10\hat{ }6 cells/mL dilution were seeded on a 384-well Microplate (culture-plate, Perkin Elmer, Cat #6007680) 24 h prior to compound testing.

Compounds were serially diluted in DMSO (Sigma, Cat #5879) and spotted on the pre-seeded cell plate, by a Labcyte Echo 55x (1% DMSO in the Assay). Incubation was for 30 min at 37° C. in a humidified incubator (rH=95%, CO2=5%). Afterwards 25 μL of 60 μM Estradiol dilution were added to the plate and incubated for 3 h at 37° C. in a humidified incubator (rH=95%, CO2=5%).

5 μL CellTiter Glo 2 (Promega, Cat #G9242) were added onto the cell plate, incubated for 15 min at room temperature and luminescence was measured on PHERAstar FSX (BMG Labtech, Ortenberg, Germany).

IC₅₀ values were calculated using a 4-parameter non-linear regression curve fitting model (Software Megalab inhouse development). For data evaluation and calculation, the measurement of the bottom (no cells, with Estradiol) was set as 0% control and the measurement of the top (includes cells and Estradiol) was set as 100% control. The IC₅₀ values were calculated using the standard 4 parameter logistic regression formula: Y═Bottom+(Top−Bottom)/(1+10\hat{ }((LogIC50−X)*Slope+log ((Top−Bottom)/(Fifty−Bottom)−1))).

### Example 8: Biochemical humanHSD17B11-RapidFire MS/MS Assay

Estradiol (Sigma, Cat #E8875), NAD (Roche, Cat #10621650001) and recombinant hHSD17B11 (U-Protein Express BV, Netherlands) were diluted in assay buffer (100 mM Tris, Sigma, Cat #T2319; sodium chloride, Roth, Cat #3957.2; 0.5 mM EDTA, Invitrogen, Cat #15575020; 0.1% TCEP, Invitrogen, Cat #T2556; 0.05% BSA fraction V (protease and fatty acid free), Serva, Cat #11945; 0,001% Tween20, Serva, Cat #37470). Compounds were serially diluted in DMSO (Sigma, Cat #5879) and spotted on a 384-well Microplate, PP, V-bottom (Greiner, Cat #781280) plate by a Labcyte Echo 55x (1% DMSO in the Assay). First, 6 μL/well of recombinant hHSD17B11 (35 nM final) dilution was added, followed by 15 min incubation at RT. Second, 6 μL/well of diluted Estradiol (30 μM final) and NAD (0.5 mM final) were added, mixed and incubated for 4 h at RT. 1 μL d4-Estrone (50 nM final; Sigma, Cat #489204) followed by 2.4 μL Girard's Reagent P (6.5 mM final; TCl, Cat #G0030) dissolved in 90% (Sigma, Cat #34860) methanol and 10% formic acid (Merck, Cat #33015) were added to derivatize analytes and stop the enzyme reaction. Incubation was for 12-24 h at RT before adding 70 μL dH2O.

The analytical sample handling was performed by a rapid-injecting RapidFire autosampler system (Agilent, Waldbronn, Germany) coupled to a triple quadrupole mass spectrometer (Triple Quad 6500, AB Sciex Germany GmbH, Darmstadt, Germany). Liquid sample was aspirated by a vacuum pump into a 10 μL. sample loop for 250 ms and subsequently flushed for 3000 ms onto a C18 cartridge (Agilent, Waldbronn, Germany) with the aqueous mobile phase (99.5% water, 0.49% acetic acid, 0.01% trifluoroacetic acid, flow rate 1.5 mL./min). The solid phase extraction step retained the analyte while removing interfering matrix (e.g., buffer components). The analyte was desorbed and eluted back from the cartridge for 3000 ms with an organic mobile phase (49.75% methanol, 49,75% acetonitrile, 0.49% acetic acid, 0.01% trifluoroacetic acid, flow rate 1.25 mL/min) and flushed into the mass spectrometer for detection in MRM mode. The MRM transition for the Estrone was 404.1<157.1 Da (declustering potential 27V, collision energy 43 V) and for the internal standard D4 Estrone was 408.1<159.1 Da (declustering potential 27V, collisionenergy 43 V). Dwell time for each MRM transition was 25 ms and pause time between MRMs is 5 ms. The mass spectrometer is operated in positive ionization mode (curtain gas 35 Au, collision gas medium, ion spray voltage 4200 V, temperature 550° C., ion source gas 1 65 Au, ion source gas 2 80 Au). While performing the back flush into the mass spectrometer, the sample loop and relevant tubing were flushed with the organic mobile phase to prevent carryover of analyte or matrix components into the next sample. Equilibration time for the system was 500 ms. To minimize carryover effects, the wash station of the RapidFire system was used to perform needle washes with pure water (100%) and pure methanol (100%) between samples. The solvent delivery setup of the RapidFire system consisted of two continuously running and isocratically operating HPLC pumps (G1310A, Agilent, Waldbronn, Germany) and one binary HPLC pump channel B (G4220A, Agilent, Waldbronn, Germany). MS data processing was performed in GMSU (Alpharetta, GA, USA), and peak area ratio analyte/internal standard was reported for IC₅₀ calculation. IC₅₀ values were calculated using a 4-parameter non-linear regression curve fitting model (Software Megalab inhouse development). For data evaluation and calculation, the measurement of the bottom (no HSD17B13 enzyme) was set as 0% control and the measurement of the top (includes NAD, Estrone and HSD17B13) was set as 100% control. The IC₅₀ values were calculated using the standard 4 parameter logistic regression formula: Y═Bottom+(Top−Bottom)/(1+10\hat{ }((LogIC50−X)×Slope+log ((Top−Bottom)/(Fifty−Bottom)−1))).

### Example 9: HumanHSD17B11-RapidFire MS/MS Assay

Estradiol (Sigma, Cat #E8875), NAD (Roche, Cat #10621650001) and recombinant hHSD17B11 (U-Protein Express BV, Netherlands) were diluted in assay buffer (100 mM Tris, Sigma, Cat #T2319; sodium chloride, Roth, Cat #3957.2; 0.5 mM EDTA, Invitrogen, Cat #15575020; 0.1% TCEP, Invitrogen, Cat #T2556; 0.05% BSA fraction V (protease and fatty acid free), Serva, Cat #11945; 0,001% Tween20, Serva, Cat #37470). Compounds were serially diluted in DMSO (Sigma, Cat #5879) and spotted on a 384-well Microplate, PP, V-bottom (Greiner, Cat #781280) plate by a Labcyte Echo 55x (1% DMSO in the Assay). First, 6 μL/well of recombinant hHSD17B11 (35 nM final) dilution was added, followed by 15 min incubation at RT. Second, 6 μL/well of diluted Estradiol (30 μM final) and NAD (0.5 mM final) was added, mixed and incubated for 4 h at RT. 1 μL d4-Estrone (50 nM final; Sigma, Cat #489204) followed by 2.4 μL Girard's Reagent P (6.5 mM final; TCl, Cat #G0030) dissolved in 90% (Sigma, Cat #34860) methanol and 10% formic acid (Merck, Cat #33015) were added to derivatize analytes and stop the enzyme reaction. Incubation was for 12-24 h at RT before adding 70 μL dH2O.

The analytical sample handling was performed by a rapid-injecting RapidFire autosampler system (Agilent, Waldbronn, Germany) coupled to a triple quadrupole mass spectrometer (Triple Quad 6500, AB Sciex Germany GmbH, Darmstadt, Germany). Liquid sample was aspirated by a vacuum pump into a 10 μL. sample loop for 250 ms and subsequently flushed for 3000 ms onto a C18 cartridge (Agilent, Waldbronn, Germany) with the aqueous mobile phase (99.5% water, 0.49% acetic acid, 0.01% trifluoroacetic acid, flow rate 1.5 mL/min). The solid phase extraction step retained the analyte while removing interfering matrix (e.g., buffer components). The analyte was desorbed and eluted back from the cartridge for 3000 ms with an organic mobile phase (49.75% methanol, 49,75% acetonitrile, 0.49% acetic acid, 0.01% trifluoroacetic acid, flow rate 1.25 mL/min) and flushed into the mass spectrometer for detection in MRM mode. The MRM transition for the Estrone was 404.1<157.1 Da (declustering potential 27V, collision energy 43 V) and for the internal standard D4 Estrone was 408.1<159.1 Da (declustering potential 27V, collisionenergy 43 V). Dwell time for each MRM transition was 25 ms and pause time between MRMs is 5 ms. The mass spectrometer is operated in positive ionization mode (curtain gas 35 Au, collision gas medium, ion spray voltage 4200 V, temperature 550° C., ion source gas 1 65 Au, ion source gas 2 80 Au). While performing the back flush into the mass spectrometer, the sample loop and relevant tubing were flushed with the organic mobile phase to prevent carryover of analyte or matrix components into the next sample. Equilibration time for the system was 500 ms. To minimize carryover effects, the wash station of the RapidFire system was used to perform needle washes with pure water (100%) and pure methanol (100%) between samples. The solvent delivery setup of the RapidFire system consisted of two continuously running and isocratically operating HPLC pumps (G1310A, Agilent, Waldbronn, Germany) and one binary HPLC pump channel B (G4220A, Agilent, Waldbronn, Germany). MS data processing was performed in GMSU (Alpharetta, GA, USA), and peak area ratio analyte/internal standard was reported for IC₅₀ calculation. IC₅₀ values were calculated using a 4-parameter non-linear regression curve fitting model (Software Megalab inhouse development). For data evaluation and calculation, the measurement of the bottom (no HSD17B13 enzyme) was set as 0% control and the measurement of the top (includes NAD, Estrone and HSD17B13) was set as 100% control. The IC₅₀ values were calculated using the standard 4 parameter logistic regression formula: Y═Bottom+(Top−Bottom)/(1+10\hat{ }((LogIC50−X)×Slope+log ((Top−Bottom)/(Fifty−Bottom)−1))).

### Example 10: Pharmacokinetic In Vitro Assay of Metabolic Stability in Liver Microsomes

The metabolic degradation of a test compound was assayed at 37° C. with pooled liver microsomes.

The final incubation volume of 60 μl per time point contained TRIS buffer pH 7.6 at RT (0.1 M), magnesium chloride (5 mM), microsomal protein (0.5-2 mg/ml) and the test compound at a final concentration of 1 μM.

Following a short pre-incubation period at 37° C., the reactions were initiated by addition of beta-nicotinamide adenine dinucleotide phosphate, reduced form (NADPH, 1 mM) and terminated by transferring an aliquot into solvent after different time points.

The quenched incubations were pelleted by centrifugation (10000 g, 5 min). An aliquot of the supernatant was assayed by LC-MS/MS for the amount of parent compound remaining.

The half-life (t1/2 INVITRO) was determined by the slope of the semi-logarithmic plot of the concentration-time profile.

The intrinsic clearance (CL_INTRINSIC) was calculated by considering the amount of protein in the incubation:

$$CL\_INTRINSIC [ μl / min / mg protein ] = ( Ln 2 / ( half - life [ min ] * [ mg / ml ] ) ) * 1000$$

### Example 11: Pharmacokinetic In Vitro Assay of Metabolic Stability in in Human Hepatocytes (HHEP Assay)

An assay in human hepatocytes was performed to assess the metabolic stability of compounds. The metabolic degradation of a test compound was assayed in a human hepatocyte suspension. After recovery from cryopreservation, human hepatocytes were diluted in DMEM (supplemented with 3.5 μg glucagon/500 ml, 2.5 mg insulin/500 ml, 3.75 mg hydrocortison/500 ml, 5% or 50% human serum or in absence of serum) to obtain a final cell density of 1.0×10⁶ cells/ml or 4.0×10⁶ cells/ml, depending on the metabolic turnover rate of the test compound.

After a 30 min preincubation in a cell culture incubator (37° C., 10% CO2) test compound solution was spiked into the hepatocyte suspension, resulting in a final test compound concentration of 1 μM and a final DMSO concentration of 0.05%.

The cell suspension was incubated at 37° C. (cell culture incubator, horizontal shaker) and samples were removed from the incubation after 0, 0.5, 1, 2, 4 and 6 hours. Samples were quenched with acetonitrile (containing internal standard) and pelleted by centrifugation. The supernatant was transferred to a 96-deepwell plate, and prepared for analysis of decline of parent compound by HPLC-MS/MS.

The percentage of remaining test compound was calculated using the peak area ratio (test compound/internal standard) of each incubation time point relative to the time point 0 peak area ratio. The log-transformed data were plotted versus incubation time, and the absolute value of the slope obtained by linear regression analysis was used to estimate in vitro half-life (T_{1/2}).

In vitro intrinsic clearance (Clᵢₙₜ) was calculated from in vitro T_{1/2} and scaled to whole liver using a hepatocellularity of 120×10⁶ cells/g liver, a human liver per body weight of 25.7 g liver/kg as well as in vitro incubation parameters, applying the following equation:

$$CL\_INTRINSIC \_INVIVO [ ml / min / kg ] = ( CL\_INTRINSIC [ μl / min / {10}^{6} cells ] \times hepatocellularity [ \text{} {10}^{6} cells / g liver ] \times liver factor [ g / kg body weight ] ) / 1000$$

Hepatic in vivo blood clearance (CL) was predicted according to the well-stirred liver model considering an average liver blood flow (QH) of 20.7 ml/min/kg:

$$CL [ ml / min / kg ] = CL\_INTRINSIC \_INVIVO [ ml / min / kg ] \times hepatic \text{} blood flow [ ml / min / kg ] / ( CL\_INTRINSIC \_INVIVO [ ml / min / kg ] + hepatic blood flow [ ml / min / kg ] )$$

Results were expressed as percentage of hepatic blood flow:

$$QH [ \% ] = CL [ ml / min / kg ] / hepatic blood flow [ ml / min / kg ]$$

### Example 12: Biological Data of the Example Compounds 1-30

| Exam- | IC₅₀ [nM], | IC₅₀ [nM], | IC₅₀ [nM], | IC₅₀ [nM], |
|---|---|---|---|---|
| ple | biochemical | biochemical | cellular | biochemical |
| com- | human | mouse | human | human |
| pound | HSD17B13 | HSD17B13 | HSD17B13 | HSD17B11 |
| 1 | 9.19 | 319 | 30.9 | 4210 |
| 2 | 26.8 | 2420 | 128 | 8610 |
| 3 | 12.2 | 2420 | 40.4 | |
| 4 | 4.86 | 446 | 76.4 | >10300 |
| 5 | 25.9 | 2560 | 116 | |
| 6 | 11.4 | 1110 | 33.5 | 8630 |
| 7 | 624 | >10300 | 1760 | >10300 |
| 8 | 59.7 | 2090 | 183 | 263 |
| 9 | 3.85 | 52.8 | 17.5 | 6090 |
| 10 | <3.09 | 63.1 | 15.3 | 8260 |
| 11 | <3.09 | 27.6 | 12.1 | 5940 |
| 12 | 0.993 | 26.8 | 15.1 | >10300 |
| 13 | 4.18 | 73.1 | 24 | >10300 |
| 14 | 3.67 | 22.4 | 23.5 | 3540 |
| 15 | 3.68 | 13.8 | 39.8 | 3260 |
| 16 | 150 | 1620 | 868 | 5900 |
| 17 | 272 | 1350 | 1280 | 8690 |
| 18 | 316 | 1710 | 1180 | 7000 |
| 19 | 12.6 | 665 | 60.6 | 1210 |
| 20 | 5.72 | 48.1 | 24.3 | 992 |
| 21 | 7.39 | 108 | 93.5 | 6320 |
| 22 | 4.45 | 25.1 | 34.4 | 5040 |
| 23 | 76.8 | 1310 | 157 | 1740 |
| 24 | 15.8 | 204 | 78.4 | 2520 |
| 25 | 1.99 | 45.9 | 19.6 | > 10300 |
| 26 | 1.26 | 23.2 | 5.13 | 10200 |
| 27 | 1.59 | 29.9 | 20.6 | 6640 |
| 28 | 12.1 | 235 | 37.4 | 1110 |
| 29 | 24.9 | 324 | 68.5 | 991 |
| 30 | 1.05 | 15.5 | <3.23 | 2260 |

1. A compound of formula (I) or a salt thereof 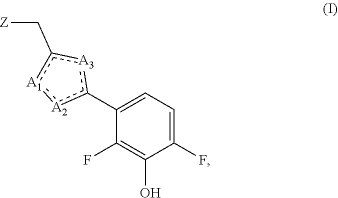
wherein
a) Z-* is selected from the group consisting of 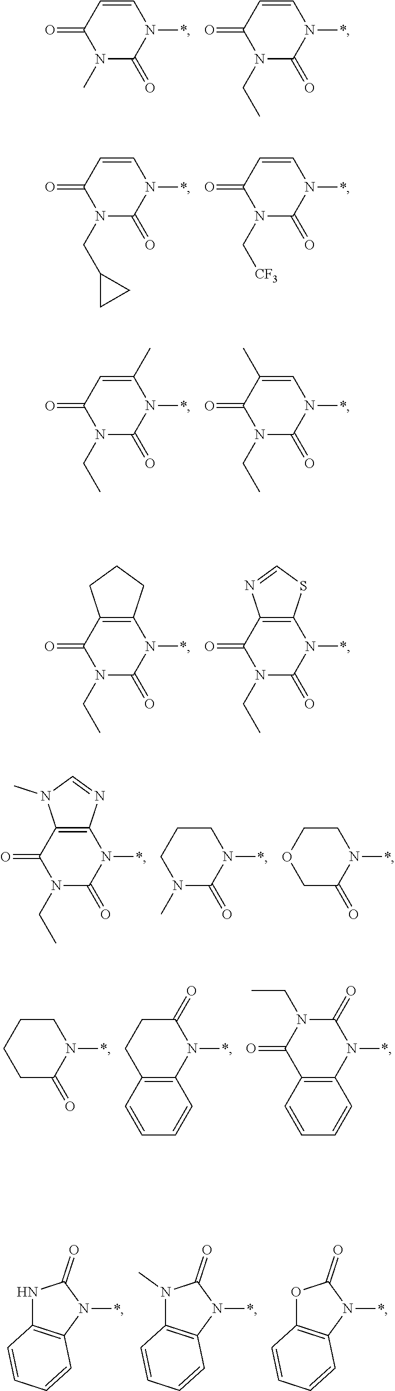 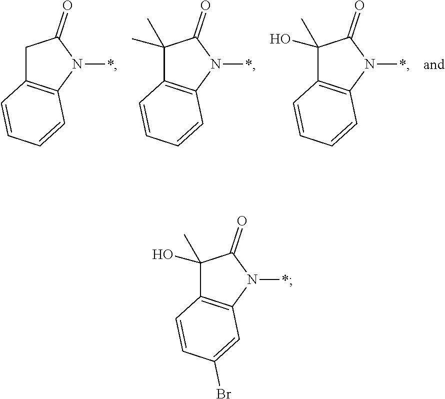
and wherein
b) the structure of 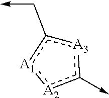
is selected from the group consisting of 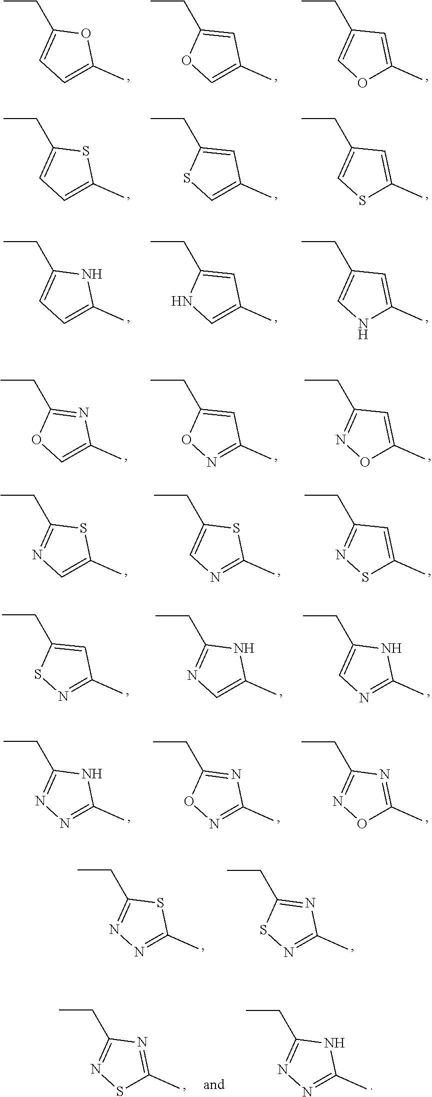
and wherein “*” denotes the point of attachment.

2. The compound of claim 1, wherein the structure of 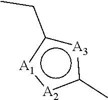
is selected from the group of structures consisting of

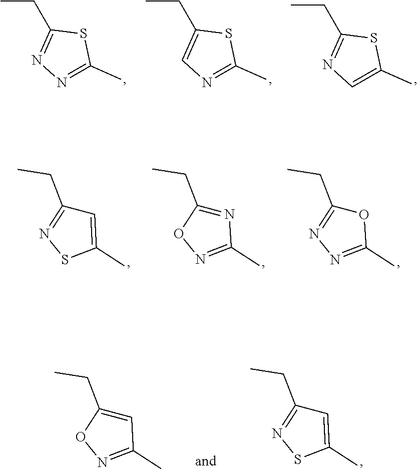

3. The compound of claim 1, wherein Z-* is selected from the group consisting of

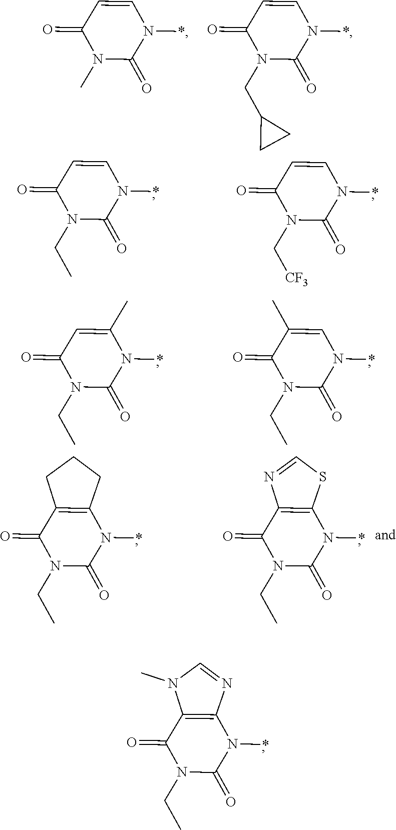

4. The compound of claim 1 or a salt thereof selected from the group of formula (I) compounds 01-30

| cpd # | Formula (I) |
|---|---|
| 01 | 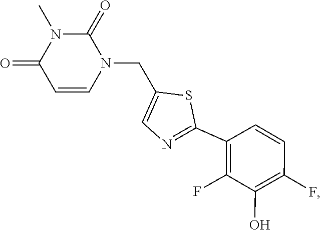 [SMILES: Cn1c(=O)ccn(Cc2cnc(-c3ccc(F)c(O)c3F)s2)c1=O] |
| 02 | 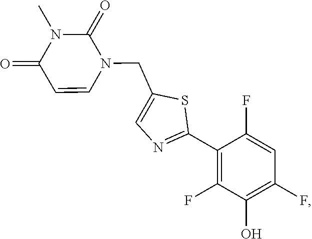 [SMILES: Cn1c(=O)ccn(Cc2cnc(-c3c(F)cc(F)c(O)c3F)s2)c1=O] |
| 03 | 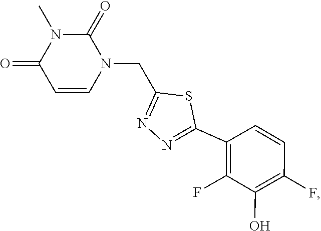 [SMILES: Cn1c(=O)ccn(Cc2nnc(-c3ccc(F)c(O)c3F)s2)c1=O] |
| 04 | 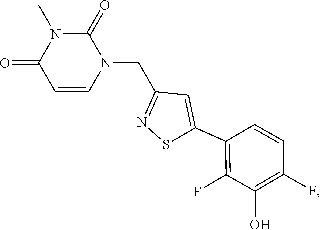 [SMILES: Cn1c(=O)ccn(Cc2cc(-c3ccc(F)c(O)c3F)sn2)c1=O] |
| 05 | 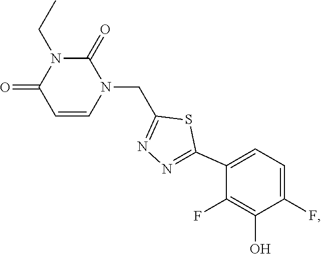 [SMILES: CCn1c(=O)ccn(Cc2nnc(-c3ccc(F)c(O)c3F)s2)c1=O] |
| 06 | 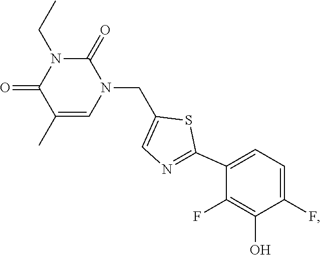 [SMILES: CCn1c(=O)c(C)cn(Cc2cnc(-c3ccc(F)c(O)c3F)s2)c1=O] |
| 07 | 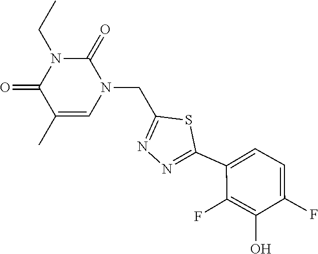 [SMILES: CCn1c(=O)c(C)cn(Cc2nnc(-c3ccc(F)c(O)c3F)s2)c1=O] |
| 08 | 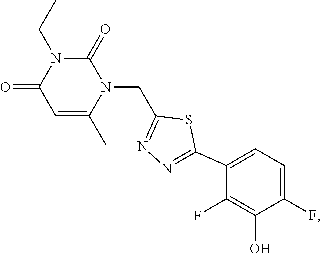 [SMILES: CCn1c(=O)cc(C)n(Cc2nnc(-c3ccc(F)c(O)c3F)s2)c1=O] |
| 09 | 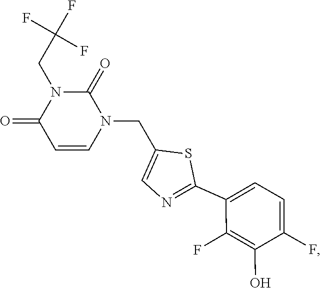 [SMILES: O=c1ccn(Cc2cnc(-c3ccc(F)c(O)c3F)s2)c(=O)n1CC(F)(F)F] |
| 10 | 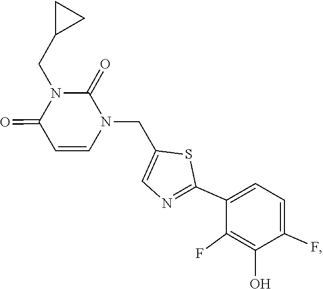 [SMILES: O=c1ccn(Cc2cnc(-c3ccc(F)c(O)c3F)s2)c(=O)n1CC1CC1] |
| 11 | 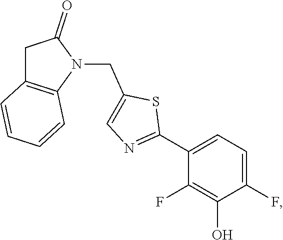 [SMILES: O=C1Cc2ccccc2N1Cc1cnc(-c2ccc(F)c(O)c2F)s1] |
| 12 | 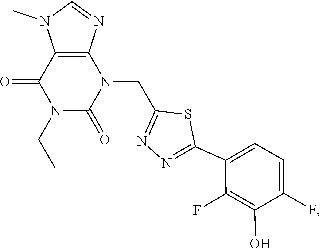 [SMILES: CCn1c(=O)c2c(ncn2C)n(Cc2nnc(-c3ccc(F)c(O)c3F)s2)c1=O] |
| 13 | 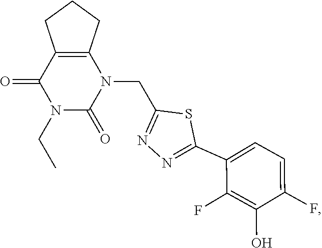 [SMILES: CCn1c(=O)c2c(n(Cc3nnc(-c4ccc(F)c(O)c4F)s3)c1=O)CCC2] |
| 14 | 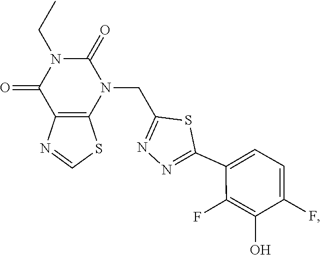 [SMILES: CCn1c(=O)c2ncsc2n(Cc2nnc(-c3ccc(F)c(O)c3F)s2)c1=O] |
| 15 | 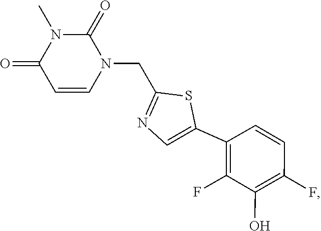 [SMILES: Cn1c(=O)ccn(Cc2ncc(-c3ccc(F)c(O)c3F)s2)c1=O] |
| 16 | 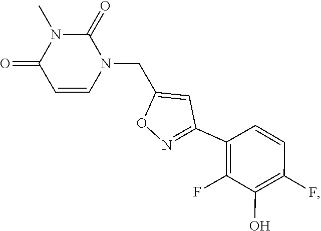 [SMILES: Cn1c(=O)ccn(Cc2cc(-c3ccc(F)c(O)c3F)no2)c1=O] |
| 17 | 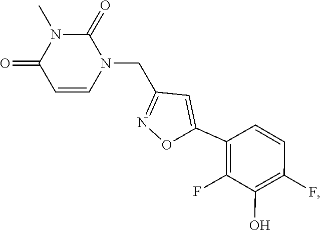 [SMILES: Cn1c(=O)ccn(Cc2cc(-c3ccc(F)c(O)c3F)on2)c1=O] |
| 18 | 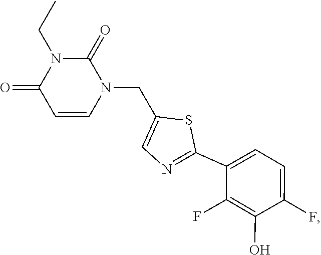 [SMILES: CCn1c(=O)ccn(Cc2cnc(-c3ccc(F)c(O)c3F)s2)c1=O] |
| 19 | 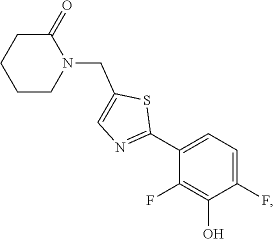 [SMILES: O=C1CCCCN1Cc1cnc(-c2ccc(F)c(O)c2F)s1] |
| 20 | 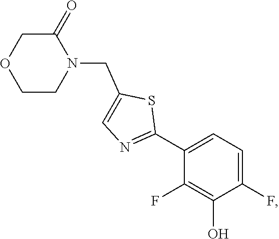 [SMILES: O=C1COCCN1Cc1cnc(-c2ccc(F)c(O)c2F)s1] |
| 21 | 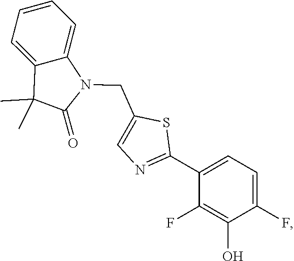 [SMILES: CC1(C)C(=O)N(Cc2cnc(-c3ccc(F)c(O)c3F)s2)c2ccccc21] |
| 22 | 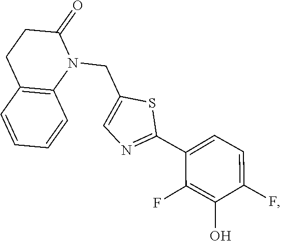 [SMILES: O=C1CCc2ccccc2N1Cc1cnc(-c2ccc(F)c(O)c2F)s1] |
| 23 | 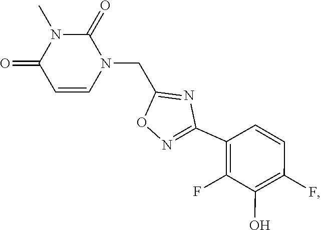 [SMILES: Cn1c(=O)ccn(Cc2nc(-c3ccc(F)c(O)c3F)no2)c1=O] |
| 24 | 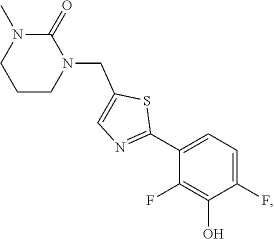 [SMILES: CN1CCCN(Cc2cnc(-c3ccc(F)c(O)c3F)s2)C1=O] |
| 25 | 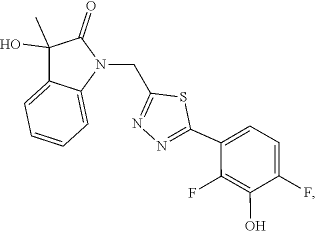 [SMILES: CC1(O)C(=O)N(Cc2nnc(-c3ccc(F)c(O)c3F)s2)c2ccccc21] |
| 26 | 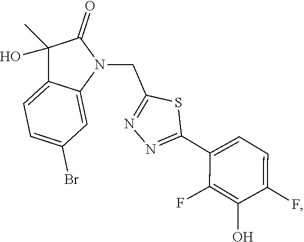 [SMILES: CC1(O)C(=O)N(Cc2nnc(-c3ccc(F)c(O)c3F)s2)c2cc(Br)ccc21] |
| 27 | 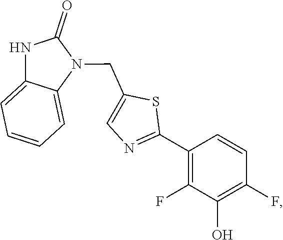 [SMILES: O=c1[nH]c2ccccc2n1Cc1cnc(-c2ccc(F)c(O)c2F)s1] |
| 28 | 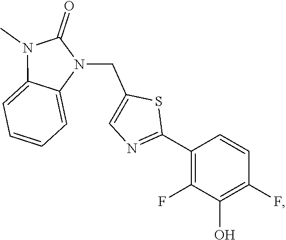 [SMILES: Cn1c(=O)n(Cc2cnc(-c3ccc(F)c(O)c3F)s2)c2ccccc21] |
| 29 | 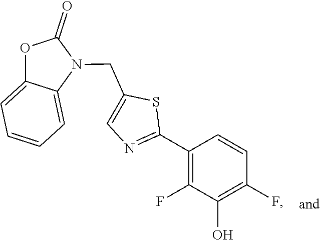 [SMILES: O=c1oc2ccccc2n1Cc1cnc(-c2ccc(F)c(O)c2F)s1] |
| 30 | 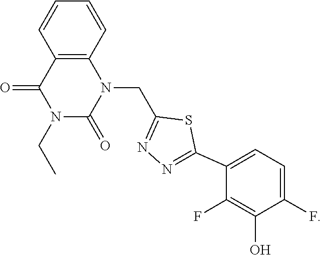 [SMILES: CCn1c(=O)c2ccccc2n(Cc2nnc(-c3ccc(F)c(O)c3F)s2)c1=O] |

5. The salt of a compound according to claim 1.

6. (canceled)

7. A method for the preparation of a compound of claim 1 of formula (I), comprising reacting compound X1, wherein Y can be Br, I or Cl, with compound X2 to give-provide compound (I)

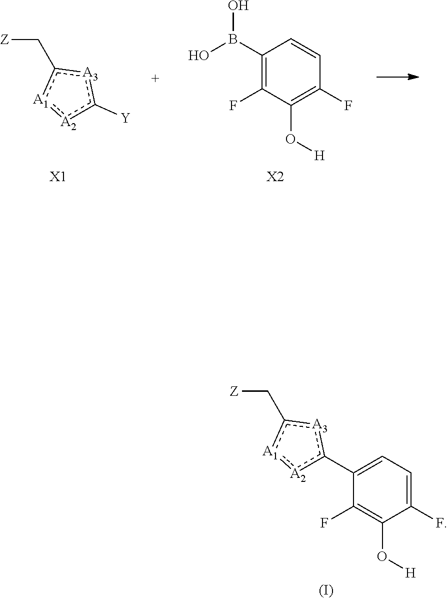

8. The method of claim 7, wherein the reaction is caried out in an aprotic or protic solvent or a solvent mixture at a temperature between 50° C. and 120° C.

9. A pharmaceutical composition comprising at least one compound according to claim 1 or a pharmaceutically acceptable salt thereof and one or more pharmaceutically acceptable excipients.

10. The pharmaceutical composition according to claim 9 comprising a therapeutically effective amount of a compound of formula (I) in the range from 0.1 to 90 wt.-% of the composition, or a pharmaceutically acceptable salt thereof.

11. A method for treating non-alcoholic fatty liver disease (NAFLD) or non-alcoholic steatohepatitis (NASH), the method comprising administering to a patient in need thereof a therapeutically effective amount of a compound according to claim 1 or a pharmaceutically acceptable salt thereof.

12. (canceled)

13. The compound of claim 1 of formula (Ia)

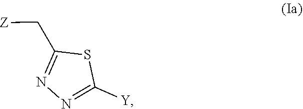

14. The compound of claim 1 of formula (Ib) [chemical structure]
